(19) United States
(12) Patent Application Publication
(10) Pub. No.: US 2017/0360920 A1
(43) Pub. Date: Dec. 21, 2017

(54) METHOD AND COMPOSITIONS FOR THE PREVENTION AND TREATMENT OF A HIV INFECTION

(71) Applicant: The Johns Hopkins University, Baltimore, MD (US)

(72) Inventors: Robert Siliciano, Baltimore, MD (US); Kai Deng, Baltimore, MD (US); Liang Shan, New Haven, CT (US)

(21) Appl. No.: 15/528,230

(22) PCT Filed: Nov. 20, 2015
(86) PCT No.: PCT/US15/61914

§ 371 Date: May 19, 2017

Related U.S. Application Data
(60) Provisional application No. 62/082,387, filed on Nov. 20, 2014.
(60) Provisional application No. 62/091,392, filed on Dec. 12, 2014.

(51) Int. Cl. A61K 39/21; C12N 7/00; A61K 39/00

(52) U.S. Cl. CPC A61K 39/21; C12N 7/00; C12N 2740/16234; C12N 2740/16171; A61K 2039/5158; A61K 2039/572; A61K 2039/545; C12N 2740/16271; C12N 2740/16134

(57) ABSTRACT

Described herein are methods of preventing or treating a HIV infection comprising administering to a mammal in need thereof, a pharmaceutically effective amount of a CD8+ T cell vaccine composition. Such methods comprising using CD8+ T cells which have been pre-stimulated with at least one HIV epitope, to thereby enhance a CD8+ T cell immune response against HIV in said mammal.

### RELATED APPLICATIONS

This Application claims the benefit of U.S. Provisional Application 62/082,387, filed Nov. 20, 2014, and U.S. Provisional Application 62/091,392, filed Dec. 12, 2014, the contents of which are incorporated herein by reference.

### BACKGROUND

Despite antiretroviral therapy (ART), HIV-1 persists in a stable latent reservoir^{1,2}, primarily in resting memory CD4⁺ T cells^{3, 4}. This reservoir presents a major barrier to the cure of HIV-1 infection. To purge the reservoir, pharmacological reactivation of latent HIV-1 has been proposed⁵ and tested both in vitro and in vivo⁶⁻⁸. A key remaining question is whether virus-specific immune mechanisms including cytolytic T lymphocytes (CTL) can clear infected cells in ART-treated patients after latency is reversed. Here we show that there is a striking all or none pattern for CTL escape mutations in HIV-1 Gag epitopes. Unless ART is started early, the vast majority (>98%) of latent viruses carry CTL escape mutations that render infected cells insensitive to CTLs directed at common epitopes. To solve this problem, we identified CTLs that could recognize epitopes from latent HIV-1 that were unmutated in every chronically infected patient tested. Upon stimulation, these CTLs eliminated target cells infected with autologous virus derived from the latent reservoir, both in vitro and in patient-derived humanized mice. The predominance of CTL-resistant viruses in the latent reservoir poses a major challenge to viral eradication. Our results demonstrate that chronically infected patients retain a broad spectrum viral-specific CTL response and that appropriate boosting of this response may be required for the elimination of the latent reservoir.

HIV-1 establishes latent infection in resting CD4⁺ T cells^{3, 4}. Recent efforts to eradicate HIV-1 infection have focused on reversing latency without global T cell activation⁵. However, inducing HIV-1 gene expression in latently infected cells is not sufficient to cause the death of these cells if they remain in a resting state⁹. Boosting HIV-1-specific immune responses including CTL responses may be required for clearance of the latent reservoir⁹. CTLs play a significant role in suppressing HIV-1 replication in acute infection¹⁰⁻¹⁴. Because of this strong selective pressure, HIV-1 quickly acquires mutations to evade CTL recognition^{12, 13, 15-18}. CTL escape has been studied primarily through the analysis of plasma virus^{12, 13, 16, 18-20}, and CTL-based vaccines have been designed based on conserved epitopes^{21, 22}. A systematic investigation of CTL escape in the latent reservoir will be of great importance to the ongoing CTL-based eradication efforts, because latent HIV-1 likely represents the major source of viral rebound after treatment interruption. Earlier studies have suggested the presence of CTL escape mutations in proviral DNA^{15, 17}, but it still remains unclear to what extent the latent reservoir in resting CD4⁺ T cells is affected by CTL escape, whether mutations detected in proviral DNA are representative of the very small fraction of proviruses that are replication-competent, and most importantly, whether the CTL response can recognize and clear infected cells after latency is reversed. Provided herein, in part, is a CD8⁺ T cell vaccine or therapy for use against latent HIV-1 infection.

### SUMMARY OF THE INVENTION

Provided herein are methods of preventing or treating a HIV infection in a mammal, such as a human. The methods involve administrating to a mammal in need thereof, a therapeutically effective amount of a CD8+ T cell vaccine composition. Such methods comprise using a CD8⁺ T cell vaccine composition which has been pre-stimulated with at least one HIV epitope, such as a HIV-1 Gag epitope, or a pool or mixture of HIV epitopes. The vaccine induces and enhances a CD8⁺ T cell immune response against HIV/AIDS in said mammal.

One aspect of the invention relates to a method of preventing or treating a HIV infection comprising administering to a mammal in need thereof, a therapeutically effective amount of a CD8⁺ T cell vaccine composition, wherein the CD8⁺ T cell has been pre-stimulated with at least one HIV epitope, to thereby enhance a CD8⁺ T cell immune response against HIV.

In certain embodiments, the pre-stimulation occurs ex-vivo.

In certain embodiments, the CD8⁺ T cell has been pre-stimulated with at least two, three, four, five, six, seven, eight, nine, or ten HIV epitopes.

In certain embodiments, the at least one HIV epitope is a subdominant epitope.

In certain embodiments, the at least one HIV epitope is a dominant epitope.

In certain embodiments, the HIV epitopes comprise subdominant and dominant epitopes.

In certain embodiments, the at least one HIV epitope is from HIV-1.

In certain embodiments, the at least one HIV epitope is selected from an epitope in the HIV-1 Gag, HIV-1 Nef, HIV-1 Rev, HIV-1 Tat, or HIV-1 Env, or combination thereof.

In certain embodiments, the at least one HIV epitope is provided in a pool or mixture of HIV epitopes.

In certain embodiments, the pool or mixture of HIV epitope is a pool or mixture of HIV-1 Gag, HIV-1 Nef, HIV-1 Rev, HIV-1 Tat, HIV-1 Env, or combination thereof.

In certain embodiments, the pool or mixture of HIV epitope is a pool or mixture of HIV-1 Gag represented by the peptide sequences set forth in Table 2, Table 4, or both.

In certain embodiments, the pool or mixture of HIV epitope is a pool or mixture of HIV-1 Nef represented by the peptide sequences set forth in Table 3.

In certain embodiments, the pool or mixture of HIV epitope is a pool or mixture of HIV-1 Rev represented by the peptide sequences set forth in Table 5.

In certain embodiments, the pool or mixture of HIV epitope is a pool or mixture of HIV-1 Tat represented by the peptide sequences set forth in Table 7.

In certain embodiments, the pool or mixture of HIV epitope is a pool or mixture of HIV-1 Env represented by the peptide sequences set forth in Table 6.

In certain embodiments, the at least one HIV epitope is an epitope in the HIV-1 Gag.

In certain embodiments, the epitope in the HIV-1 Gag is selected from any one of SEQ ID NOs: 1-17, or combination thereof.

In certain embodiments, the HIV-1 Gag is from proviral HIV-1 DNA in resting CD4+ T cells from mammals during the acute phase or chronic phase of infection.

In certain embodiments, the at least one HIV epitope is synthetic.

In certain embodiments, the at least one HIV epitope is unmutated.

In certain embodiments, the at least one HIV epitope is mutated.

In certain embodiments, the CD8⁺ T cell immune response is to subdominant epitope in HIV-1.

In certain embodiments, the CD8⁺ T cell is from CP36 or CP39.

In certain embodiments, the CD8⁺ T cell is autologous.

In certain embodiments, the CD8⁺ T cell has been pre-stimulated with at least one HIV epitope and at lease one cytokine.

In certain embodiments, the at least one cytokine is interleukin-2 (IL-2).

In certain embodiments, the CD8⁺ T cell to CD4⁺ T cell ratio is enhanced.

In certain embodiments, the CD8⁺ T cell response targets latent or reactivated HIV-1 infected cells.

In certain embodiments, the CD8⁺ T cell immune response is greater in magnitude than a CD8⁺ T cell immune response induced by administration of an unstimulated CD8⁺ T cell composition.

In certain embodiments, the CD8⁺ T cell immune response is greater in magnitude than a CD8⁺ T cell immune response induced by administration of the HIV epitope alone.

In certain embodiments, the efficacy of the immune response against HIV results in a reduction of the levels of HIV viral replication.

In certain embodiments, the reduction of levels of HIV viral replication is decreased in log₁₀ reductions of about 2-logs, 3-logs, 4-logs, 5-logs, 6-logs, 7-logs, 8-logs, or 9-logs.

In certain embodiments, the efficacy of the immune response against HIV results in a reduction of levels of plasma HIV-1 RNA.

In certain embodiments, the reduction of the levels of plasma HIV-1 RNA is in log₁₀ reductions of about 2-logs, 3-logs, 4-logs, 5-logs, 6-logs, 7-logs, 8-logs, or 9-logs.

In certain embodiments, the efficacy of the immune response against HIV results in a reduction of levels of proviral HIV-1 DNA.

In certain embodiments, the levels of provrial HIV-1 DNA is decreased 100-, 200-, 300-, 400-, 500-, 600-, 700-, 800-, 900-, 1000-, 1500-, or 2000-fold.

In certain embodiments, the efficacy of the immune response against HIV results in a reduction of the HIV-1 latent reservoir.

In certain embodiments, the HIV-1 latent reservoir is decreased 100-, 200-, 300-, 400-, 500-, 600-, 700-, 800-, 900-, 1000-, 1500-, or 2000-fold when compared to the resting CD4⁺ T cell population in any healthy or infected individual or total latently infected resting CD4⁺ T cell population.

In certain embodiments, the resting CD4⁺ T cell population in any healthy or infected individual about 10¹² cells.

In certain embodiments, the total latently infected resting CD4+T cell population is from about 10⁶ to about 10⁷ cells.

In certain embodiments, the efficacy of the immune response against HIV results in a delay in rebound of HIV viremia after cessation of antiretroviral therapy.

In certain embodiments, the delay is measured in months of about 1, 2, 3, 4, 5, 6, 7, 8, 9, 10, or 11 months.

In certain embodiments, the mammal is a human.

In certain embodiments, the human is afflicted with HIV-1.

In certain embodiments, the human is chronically infected with HIV-1.

In certain embodiments, the human is acutely infected with HIV-1.

In certain embodiments, the CD8⁺ T cell vaccine composition is administered to a human on suppressive antiretroviral therapy.

In certain embodiments, the CD8⁺ T cell vaccine composition is administered to the antiretroviral-treated human followed by antiretroviral treatment interruption.

In certain embodiments, the CD8⁺ T cell vaccine composition is administered to the antiretroviral-treated human in combination with latency reversing therapy.

In certain embodiments, the composition is administered to the mammal more than one time over the course of treating or preventing.

In certain embodiments, the composition is administered to the mammal in need thereof at about weeks two, three, four, five, six, seven, eight, nine, ten, eleven, twelve, thirteen, fourteen, fifteen, and sixteen post-HIV infection.

In certain embodiments, the therapeutically effective amount is about 10², 10³, 10⁴, 10⁵, 10⁶, 10⁷, 10⁸, 10⁹, 10¹⁰, 10¹¹, or 10¹² prestimulated CD8⁺ T cells per infusion into a patient.

In certain embodiments, the effective amount is between about 10⁷ to 10⁹ prestimulated CD8⁺ T cells per infusion into a patient.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1a-1e. CTL escape variants dominate the latent reservoir of CP-treated but not AP-treated patients. a, Frequency of variants in Gag CTL epitopes in proviruses from resting CD4⁺ T cells. Representative results of 6 patients are shown. Only optimal CTL epitopes relevant to each patient's HLA type are listed. Results from both Pacbio (left bar) and MiSeq (right bar) sequencing are shown. The effect on CTL recognition (denoted by color) is determined from information in the Los Alamos National Laboratory (LANL) HIV Molecular Immunology Database. b, CTL escape variants identified by sequencing are specific to HLA type. Frequencies of documented escape-associated variants in four well-characterized epitopes are shown for all 15 CP-treated patients. Median and P value from Mann-Whitney test are shown. c, Comparison of CTL escape variant frequency in proviruses between CP- and AP-treated patients. Only well-characterized epitopes are shown. Median and P value from Mann-Whitney test are shown. d, Characterization of CTL responses against HIV-1 Gag epitopes by interferon-γ ELISpot. The peptides tested are listed below the x-axis (black type, epitopes in which sequence variation was detected; blue type, no variation). The observed mutation is underlined in red, and CTL escape (defined by the lost of positive response) is denoted by * above the bar. The peptide concentration was 10 μg/ml. Error bars represent s.e.m., n=3. e, Sequences in Gag CTL epitopes for proviral DNA and outgrowth virus from resting CD4⁺ T cells in CP39. CTL epitopes with no observed variation are highlighted in blue. Epitopes with documented escape mutations are shaded in red.

FIGS. 2a-2c. CD8⁺ T cells pre-stimulated with a mixture of Gag peptides eliminate autologous CD4⁺ T cells infected with autologous HIV-1 from resting CD4⁺ T cells. a, Pre-stimulated CD8⁺ T cells (sCD8) eliminate autologous infected CD4⁺ T cells more efficiently than unstimulated CD8⁺ T cells (uCD8). Each symbol represents the mean of 3 replicates. Median and P value from Mann-Whitney test are shown. b, sCD8 inhibit viral growth in autologous infected CD4⁺ T cells with higher efficacy than uCD8. c, sCD8 pre-stimulated by different viral peptides eliminate autologous CD4⁺ T cells infected with viruses derived from resting CD4⁺ T cells. For b and c, results are compared with CD4 only using paired t tests. Error bars represent s.e.m., n=3. *: p<0.05; **: p<0.01; ***: p<0.001; ns: p>0.05.

FIGS. 3a-3d. CD8⁺ T cells targeting unmutated epitopes, not epitopes with identified escaped mutations, eliminate CTL escape variants. a, Frequency of variants in Gag CTL epitopes in proviruses from resting CD4⁺ T cells of CP36 and CP39. Epitopes tested with single peptide stimulation herein are denoted in colors (red or pink, epitopes with escape observed; blue, no escape observed). b, Epitope-specific CD8⁺ T cells proliferate significantly after single peptide stimulation. Only CD8⁺ cells are shown. Percentages of CFSE_{low}, pentamer-positive cells are indicated for unstimulated cultures (uCD8) with or without IL2, for cultures stimulated with Gag peptide mixture (sCD8) and IL2, and for cultures stimulated with the indicated single peptides and IL2. Wild type versions of peptides were used for all single peptide stimulations. c, CD8⁺ T cell proliferative responses after single peptide stimulation. Only CD8⁺ cells are shown. Percentages of CFSE_{low} cells are indicated. d, CD8⁺ T cells targeting unmutated epitopes, not epitopes with identified escaped mutations, eliminate autologous CD4⁺ T cells infected with CTL escape variants. uCD8: unstimulated CD8⁺ T cells; sCD8: Gag peptide mixture stimulated CD8⁺ T cells. Error bars represent s.e.m., n=3. *: p<0.05; **: p<0.01; ***: p<0.001; ns: p>0.05, paired t test.

FIGS. 4a-4h. Broad-spectrum cytotoxic T lymphocytes suppress in vivo replication of HIV-1 from the latent reservoir of the same patients in patient-derived humanized mice. a, Experimental design. b and c, Efficient engraftment of patient CP18-derived hematopoietic cells in MIS(^{K}I)TRG mice at week 6. Representative flow cytometry analysis (b) and summary (c) of human CD45⁺ cells, human T-lymphocyte and monocyte subsets. 11 out of 15 mice (enclosed in the rectangle) were used for HIV-1 infection. d, Correlation between frequency of peripheral human CD45⁺ cells (6 weeks after engraftment) and plasma HIV-1 RNA levels (14 days after infection). e, Depletion of peripheral CD4⁺ T cells after HIV-1 infection. *: p<0.05, paired t-test, n=11. f and g, Reduction of levels of plasma HIV-1 RNA and copies of peripheral blood HIV-1 DNA after injection of viral-specific CTLs. Filled: above detection limit; open: below detection limit. *: p<0.05, unpaired t test. h, Effect of CTL on the level of viral replication in vivo. The area under the curve of the viremia vs. time plot for each mouse in f and g before (AUC1) or after (AUC2) injection of CD8⁺ T cells was calculated to quantitatively represent viral replication over time. *: p<0.05, unpaired t test.

FIG. 5. CTL escape variants dominate the latent reservoir of CP-treated HIV-1-positive individuals, but not AP-treated individuals. Frequency of variants in Gag CTL epitopes in proviruses from resting CD4⁺ T cells. Results of all 25 patients tested are shown. Only optimal CTL epitopes relevant to each patient's HLA type are listed in linear positional order on the X axis. Results from both Pacbio (left bar) and MiSeq (right bar) sequencing platforms are shown for each epitope. The absence of bars above a listed epitope indicates that only wild type sequences were detected. For each mutation in a CTL epitope, information regarding the effect of the mutation on CTL recognition from the Los Alamos National Laboratory (LANL) HIV Molecular Immunology Database or from ELISpot assays described herein was used to assign the mutation to one of the categories indicated at the bottom. See Methods for definitions of these categories.

FIG. 6. Characterization of CTL responses against HIV-1 Gag epitopes by interferon-γ ELISpot. Results of 7 patients tested are shown. The peptides tested are listed for each patient in each graph. Error bars represent s.e.m., n=3.

FIG. 7. Partial Gag sequences from proviral DNA and outgrowth virus from resting CD4⁺ T cells of 8 CP-treated patients (following FIG. 1e). CTL epitopes with no observed variation are highlighted in blue. Documented escape mutations (red shading), inferred escape mutations (yellow shading), diminished response (pink shading), susceptible form (green shading) or undetermined variations (gray shading) in relevant optimal epitopes are indicated. See Methods for definitions of these types of mutations.

FIGS. 8a-8d. CD8⁺ T cells pre-stimulated with a mixture of consensus B Gag peptides eliminate autologous CD4⁺ T cells infected with autologous HIV-1 from resting CD4⁺ T cells. a, HIV-1 isolated from ART-treated individuals replicates as well as lab strain virus BaL. p24 values represent mean of 3 replicates. Error bars represent s.e.m., n=3. b, CD8⁺ T cells are not stimulated after co-cultured with PHA-activated CD4⁺ T cells. c, A representative flow cytometric analysis of CTL-mediated killing after co-culture of infected CD4⁺ T cells with autologous CD8⁺ T cells. CTL activity is measured by the percentage of Gag-positive, CD8-negative cells after 3 days of co-culture relative to cultures without CD8⁺ T cells. d, Pre-stimulated CD8⁺ T cells eliminate autologous infected CD4⁺ T cells more efficiently than non-stimulated CD8⁺ T cells. All results were normalized to the CD4 only control group. Error bars represent s.e.m., n=3. *: p<0.05; **: p<0.01; ***: p<0.001; ns: p>0.05, paired t test.

FIGS. 9a-9b. The elimination of infected CD4⁺ T cells is mediated by direct killing by autologous CD8⁺ T cells. a, Killing of infected CD4⁺ T cells is enhanced by increased E:T ratios for both pre-stimulated and non-stimulated CD8⁺ T cells. b, Killing of the infected CD4⁺ T cells depends on direct cell-cell contact between CD4⁺ T cells and CTLs. All results were normalized to the CD4 only control group. Error bars represent s.e.m., n=3. *: p<0.05; **: p<0.01; ***: p<0.001; ns: p>0.05, paired t test.

FIGS. 10a-10d. Viral dynamics and depletion of CD4⁺ T cells in humanized mice. a, Viral dynamics in CP18-infected MIS^{(KI)}TRG mice. CP18-derived MIS^{(KI)}TRG mice were infected with autologous HIV-1. Plasma HIV-1 RNA levels were measured from day 0 to day 56. b, Depletion of CD4 T cells in peripheral blood of HIV-1 BaL-infected mice. MIS^{(KI)}TRG mice engrafted with fetal liver CD34 cells were infected with HIV-1 BaL. CD4 to CD8 ratio in peripheral blood was measured by FACS from day 0 to day 29 after infection. c, Depletion of CD4 T cells in spleen of HIV-1 BaL-infected mice. MIS^{(KI)}TRG mice engrafted with fetal liver CD34 cells were infected with HIV-1 BaL. CD4 to CD8 ratio in spleen was measured by FACS 20 days after infection. Median and P value from Mann-Whitney test are shown. d, Detection of cell-associated HIV-1 RNA in T cells and macrophages/monocytes. CD3⁺ and CD14⁺ human cells from HIV-1-infected MIS^{(KI)}TRG mice from spleen and lung were purified by FACS. CD3⁻CD14⁻ cells were also collected as controls. Cell associated HIV-1 RNA was quantified by gag-specific qPCR. *: p<0.05, unpaired t test.

FIGS. 11a-11c. HIV-1 infection occurs in peripheral blood and tissues in humanized mice. a, Engraftment levels of MIS^{(KI)}TRG mice with fetal liver or patient CD34 cells. b, Memory CD4⁺ T cells are detected in MIS^{(KI)}TRG mice after infection. MIS^{(KI)}TRG mice were infected with HIV-1 BaL. Peripheral blood and indicated tissues from infected mice were collected at 20 dpi. Memory CD4 T cells were determined by CD45RO staining. c, Total number of cell associated HIV-1 DNA in blood and tissues. DNA Leukocytes from peripheral blood or indicated tissues were isolated for the measurement of total amount of cell-associated HIV-1 DNA by real-time PCR. For b and c, median and P value from Mann-Whitney test are shown.

FIG. 12. Broad-spectrum cytotoxic T lymphocytes suppress in vivo infection of patient-derived humanized mice with autologous latent HIV-1. Generation of patient CP36-derived humanized mice was described in FIG. 4. Mice were infected with autologous viruses at 6 weeks old. CD8 T cells from CP36 were pre-stimulated with the mixture of Gag peptides or left untreated for 6 days in vitro and were injected into mice by i.v. 9 days after infection. Plasma HIV-1 RNA and HIV-1 DNA in peripheral blood were measured by real-time PCR.

### DETAILED DESCRIPTION

### A. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “administering” includes any method of delivery of a compound of the present invention, including but not limited to, a pharmaceutical composition, therapeutic agent, or CD8⁺ T cell vaccine composition into a subject's system or to a particular region in or on a subject. The phrases “systemic administration,” “administered systemically,” “peripheral administration,” “administered peripherally,” “infusion,” and “reinfusion” as used herein mean the administration of a compound, drug, CD8⁺ T cell vaccine composition, or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “amino acid” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

Also included are the (d) and (l) stereoisomers of such amino acids when the structure of the amino acid admits of stereoisomeric forms. The configuration of the amino acids and amino acid residues herein are designated by the appropriate symbols (d), (l) or (dl). Furthermore, when the configuration is not designated the amino acid or residue can have the configuration (d), (l) or (dl). It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (d) or (l) stereoisomers.

The terms “comprise” and “comprising” are used in the inclusive, open sense, meaning that additional elements may be included.

The term “HIV” is known to one skilled in the art to refer to Human Immunodeficiency Virus. There are two types of HIV: HIV-1 and HIV-2. There are many different strains of HIV-1. The strains of HIV-1 can be classified into three groups: the “major” group M, the “outlier” group O and the “new” group N. These three groups may represent three separate introductions of simian immunodeficiency virus into humans. Within the M-group there are at least ten subtypes or clades: e.g., clade A, B, C, D, E, F, G, H, I, J, and K. A “clade” is a group of organisms, such as a species, whose members share homologous features derived from a common ancestor. Any reference to HIV-1 in this application includes all of these strains.

The term “including” is used to mean “including but not limited to”. “Including” and “including but not limited to” are used interchangeably.

A “patient” or “subject” or “mammal” refers to either a human or non-human animal.

The term “pharmaceutical delivery device” refers to any device that may be used to administer a therapeutic agent or agents to a subject. Non-limiting examples of pharmaceutical delivery devices include hypodermic syringes, multichamber syringes, stents, catheters, transcutaneous patches, microneedles, microabraders, and implantable controlled release devices.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polypeptide”, “peptide” and “epitope” are used interchangeably herein to refer to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

In certain embodiments, peptides of the invention may be synthesized chemically, ribosomally in a cell free system, or ribosomally within a cell. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol.

245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

As known to one skilled in the art, “retroviruses” are diploid positive-strand RNA viruses that replicate through an integrated DNA intermediate (proviral DNA). In particular, upon infection by the RNA virus, the lentiviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells. The retrovirus genome contains at least three genes: Gag codes for core and structural proteins of the virus; Pol codes for reverse transcriptase, protease and integrase; and Env codes for the virus surface proteins. Within the retrovirus family, HIV is classified as a lentivirus, having genetic and morphologic similarities to animal lentiviruses such as those infecting cats (feline immunodeficiency virus), sheep (visna virus), goats (caprine arthritis-encephalitis virus), and non-human primates (simian immunodeficiency virus).

### B. Methods of Preventing or Treating a HIV Infection

Provided are methods of preventing or treating a lentiviral infection, such as a HIV infection, comprising administering to a mammal in need thereof, a therapeutically effective amount of a CD8⁺ T cell vaccine composition, wherein the CD8⁺ T cell has been pre-stimulated with at least one HIV epitope, to thereby enhance a CD8⁺ T cell immune response against HIV.

The term “effective amount” as used herein means an amount effective and at dosages and for periods of time necessary to achieve the desired result. The term “mammal” as used herein includes all members of the animal kingdom including non-humans and humans. In certain embodiments, the mammal may be a human. The human may be afflicted with HIV-1. The human may be chronically infected or acutely infected with HIV-1. The human may be on suppressive antiretroviral therapy. In certain embodiments, the pre-stimulated CD8+ T cell vaccine composition can be reinfused only in antiretroviral therapy (ART)-treated individuals, reinfused only in untreated individuals, reinfused only in ART-treated individuals, followed by antiretroviral treatment interruption, or reinfused combined with latency reversing therapy in ART-treated individuals.

In certain embodiments, the CD8⁺ T cell vaccine composition is administered to the patient more than one time over the course of treating or preventing.

In certain embodiments, the efficacy of the CD8⁺ T cell vaccine composition may relate to HIV latency and the ability to remain off of antiretroviral therapy without HIV rebound. In certain embodiments, these measures include reduction in proviral DNA. The estimated number of HIV proviruses per infected person on therapy are 10⁸ to 10⁹. In certain embodiments, the reductions in proviral DNA that result in a delay in rebound may be on the order of about 100- to 1000-fold reductions. In other embodiments, these measures included reduction in the total number of resting CD4⁺ T cells in any healthy or infected individual would be approximately 10¹² using well-known methods in the art of measuring reservoir size. In other embodiments, these measures included reduction in the total latently infected resting CD4 T cell population appears to be between 10⁶ and 10⁷ cells. In certain embodiments, a 1000-fold reduction would result in significant delay in rebound when off antiretroviral therapy. In other embodiments, efficacy is measured in delay in rebound of HIV viremia after cessation of antiretroviral therapy measured in months.

### C. Therapeutically Effective CD8⁺ T Cell Vaccine Compositions

A therapeutically effective amount of a CD8⁺ T cell vaccine composition comprises CD8⁺ T cells which have been pre-stimulated with at least one HIV epitope, to thereby enhance a CD8⁺ T cell immune response.

The pre-stimulation occurs ex-vivo and may include incubating the CD8⁺ T cells with at least one cytokine in addition to the at least one HIV epitope. The CD8⁺ T cell may be derived from CP36 or CP39. The CD8⁺ T cell may be autologous to the mammal.

The HIV epitopes may include at least 2, 3, 4, 5, 6, 7, 8, 9, 10, 15, 20, 30, 40, 50, 60, 70, 80, 90, 100, 125, 150, 175, 200, 225, 250, 275, 300, 325, 350, 375, 400, 425, 450, 475, or 500 HIV epitopes. The at least one HIV epitope may be a subdominant or dominant epitope. The at least two HIV epitopes may comprise subdominant and dominant epitopes.

In certain embodiments, the CD8⁺ T cell response may target latent or reactivated HIV-1 infected cells. In certain embodiments, the CD8⁺ T cell immune response may be greater in magnitude than a CD8⁺ T cell immune response induced by administration of an unstimulated CD8⁺ T cell composition. In other embodiments, the CD8⁺ T cell immune response is greater in magnitude than a CD8⁺ T cell immune response induced by administration of the HIV epitope alone. The CD8⁺ T cell immune response may be in response to a subdominant epitope(s) in HIV-1.

In certain embodiments, the at least one HIV epitope may be from HIV-1. In certain embodiments, the at least one HIV epitope may be from HIV-2. In certain embodiments, the at least one HIV epitope may be selected from an epitope in the HIV-1 Gag, HIV-1 Nef, HIV-1 Rev, HIV-1 Tat, or HIV-1 Env. In certain embodiments, the HIV epitope may be provided singly, or in combination as a mixture or pool of HIV peptides. In certain embodiments, the pool or mixture of HIV peptides are defined by the Gag peptides in Table 2 and Table 4 (i.e., NIH AIDS Reagent 8117). In certain embodiments, the pool or mixture of HIV peptides are defined by the Nef peptides in Table 3 (i.e., NIH AIDS Reagent 5189). In certain embodiments, the pool or mixture of HIV peptides are defined by the Rev peptides in Table 5 (i.e., NIH AIDS Reagent 6445). In certain embodiments, the pool or mixture of HIV peptides are defined by the Tat peptides in Table 7 (i.e., NIH AIDS Reagent 5138). In certain embodiments, the pool or mixture of HIV peptides are defined by the Env peptides in Table 6 (i.e., NIH AIDS Reagent 9480). Said mixtures or pools of HIV peptides may comprise different combinations of the HIV peptides set for the in Tables 2-7. In certain embodiments, the at least one HIV epitope is an epitope in HIV-1 Gag. In certain embodiments, the HIV-1 Gag epitopes may be selected from ISPRTLNAW (SEQ ID NO: 1), LSPRTLNAW (SEQ ID NO: 2), TSTLQEQIGW (SEQ ID NO: 3), TSNLQEQIGW (SEQ ID NO: 4), QASQEVKNW (SEQ ID NO: 5), QSTQEVKNW(SEQ ID NO: 6), KAFSPEVIPMF (SEQ ID NO: 7), SLYNTVATL (SEQ ID NO: 8), SLFNTVAVL (SEQ ID NO: 9), WASRELERF (SEQ ID NO: 10), TLNAWVKVV (SEQ ID NO: 11), RLRPGGKKK (SEQ ID NO: 12), RLRPGGKKS (SEQ ID NO: 13), LYNTVATLY (SEQ ID NO: 14), LFNTIAALF (SEQ ID NO: 15), TPQDLNTML (SEQ ID NO: 16), or GPGHKARVL (SEQ ID NO: 17). In certain embodiments, the HIV Env epitope is found in the consensus Subtype B sequence as follows:

(SEQ ID NO: 18)

MRVKGIRKNYQHLWRWGTMLLGMLMICSAAEKLWVTVYYGVPVWKEATTT
LFCASDAKAYDTEVHNVWATHACVPTDPNPQEVVLENVTENFNMWKNNMV
EQMHEDIISLWDQSLKPCVKLTPLCVTLNCTDLMNATNTTNSSSGEKMEK
GEIKNCSFNITTSIRDKVQKEYALFYKLDVVPIDNDNTSSYRLISCNTSV
ITQACPKVSFEPIPIHYCAPAGFAILKCNDKKFNGTGPCTNVSTVQCTHG
IRPVVSTQLLLNGSLAEEEVVIRSENFTNNAKTIIVQLNESVEINCTRPN
NNTRKSIHIGPGRAFYTTGEIIGDIRQAHCNISRAKWNNTLKQIVKKLRE
QFGNKTIVFNQSSGGDPEIVMHSFNCGGEFFYCNTTQLFNSTWNVNGTWN
NNTEGNDTITLPCRIKQIINMWQEVGKAMYAPPIRGQIRCSSNITGLLLT
RDGGNNNTNETEIFRPGGGDMRDNWRSELYKYKVVKIEPLGVAPTKAKRR
VVQREKRAVGIGAMFLGFLGAAGSTMGAASMTLTVQARQLLSGIVQQQNN
LLRAIEAQQHLLQLTVWGIKQLQARVLAVERYLKDQQLLGIWGCSGKLIC
TTTVPWNASWSNKSLDEIWDNMTWMEWEREIDNYTSLIYTLIEESQNQQE
KNEQELLELDKWASLWNWFDITNWLWYIKIFIMIVGGLIGLRIVFAVLSI
VNRVRQGYSPLSFQTRLPAPRGPDRPEGIEEEGGERDRDRSGRLVDGFLA
LIWDDLRSLCLFSYHRLRDLLLIVTRIVELLGRRGWEVLKYWWNLLQYWS
QELKNSAVSLLNATAIAVAEGTDRVIEVVQRACRAILHIPRRIRQGLERA
LL.

In certain embodiments, the HIV epitope may be synthetic and may be chemically synthesized as described in section “A” above. In other embodiments, the HIV epitopes may be mutated or unmutated. Other HIV epitopes may be deep sequences from from proviral HIV-1 DNA in resting CD4⁺ T cells from mammals during the acute phase or chronic phase of infection (see examples section of instant specification).

The present invention further features methods comprising the administration of an effective amount a CD8⁺ T cell vaccine composition, wherein the composition comprises CD8⁺ T cells which have been pre-stimulated with at least one HIV epitope, or a pool or mixure of HlVepitodes set forth in Tables 2-7, to thereby enhance a CD8⁺ T cell immune response, as described above. Dosage levels of between about 10², 10³, 10⁴, 10⁵, 10⁶, 10⁷, 10⁸, 10⁹, 10¹⁰, 10¹¹, or 10¹² cells per infusion into a patient may be useful as a vaccine injection in the methods described herein. The amount of active ingredient that may be combined with the carrier materials to produce a single dosage form will vary depending upon the host treated and the particular mode of administration. The dose of the vaccine may vary according to factors such as the infection state, age, sex, and weight of the individual, and the ability of CD8⁺ T cell vaccine composition to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative or treatment dose response depending upon the circumstances.

In certain embodiments, the dosage may enhance the CD8⁺ T cell to CD4⁺ T cell ratio. The efficacy of the prevention or treatment of the methods of the present invention may be determined from samples obtained from the mammal after treatment has began using the following. In certain embodiments, the efficacy is determined comparing a sample of a mammal obtained during the course of treatment to a sample which has been previously obtained from the patient, such as at the start of treatment or in an initial sample obtained two, three, or four weeks post HIV infection but prior to treatment. In certain embodiments, the levels of HIV viral replication is decreased in a plasma sample when compared to a plasma sample previously obtained from the mammal prior to initiation of treatment. The levels of HIV viral replication may be decreased in terms of log₁₀ reductions of about 2-logs, 3-logs, 4-logs, 5-logs, 6-logs, 7-logs, 8-logs, or 9-logs. In certain embodiments, the levels of plasma HIV-1 RNA may be decreased in a plasma sample when compared to a plasma sample previously obtained from the mammal prior to initiation of treatment. The levels of plasma HIV-1 RNA may be decreased in terms of log₁₀ reductions of about 2-logs, 3-logs, 4-logs, 5-logs, 6-logs, 7-logs, 8-logs, or 9-logs. In other embodiments, the levels of proviral HIV-1 DNA may be decreased 100-, 200-, 300-, 400-, 500-, 600-, 700-, 800-, 900-, 1000-, 1500-, or 2000-fold. The fold reduction may be calculated as a reduction from an estimated 10⁸ or 10⁹ HIV provirus per infected person on antiretroviral therapy. In other embodiments, the efficacy of the CD8⁺ T cell vaccine composition can be determined when the HIV-1 latent reservoir is decreased 100-, 200-, 300-, 400-, 500-, 600-, 700-, 800-, 900-, 1000-, 1500-, or 2000-fold when compared to the resting CD4+ T cell population in any healthy or infected individual or total latently infected resting CD4+ T cell population using well known methods in the art to measure the reservoir size. In certain embodiments, the resting CD4⁺ T cell population in any healthy or infected individual is about 10¹² cells. In certain embodiments, the total latently infected resting CD4⁺ T cell population is from about 10⁶ to about 10⁷ cells. Such fold reductions may result in significant delay in rebound when off of antiretroviral therapy. In other embodiments, the efficacy of the CD8⁺ T cell vaccine composition relate to HIV latency and the ability of the patient to remain off of antiretroviral therapy without HIV rebound. In certain embodiments, efficacy of the CD8⁺ T cell vaccine composition can be determined by the delay in rebound of HIV viremia after cessation of antiretroviral therapy. Typically, a rebound of HIV viremia may occur in a couple of weeks in patients who have stopped antiretroviral therapy. In certain embodiments a significant delay may be measured in months of about 1, 2, 3, 4, 5, 6, 7, 8, 9, 10, or 11 months.

The compositions of the invention are suitable for administration to subjects in a biologically compatible form in vivo. The expression “biologically compatible form suitable for administration in vivo” as used herein means a form of the substance to be administered in which any toxic effects are outweighed by the therapeutic effects. The substances may be administered to any animal, preferably humans.

The CD8⁺ T cell vaccine composition of the present invention may additionally contain suitable diluents, adjuvants and/or carriers. Preferably, the vaccines contain an adjuvant which can enhance the immunogenicity of the vaccine in vivo. The adjuvant may be selected from many known adjuvants in the art including the lipid-A portion of gram negative bacteria endotoxin, trehalose dimycolate of mycobacteria, the phospholipid lysolecithin, dimethyldictadecyl ammonium bromide (DDA), certain linear polyoxypropylene-polyoxyethylene (POP-POE) block polymers, aluminum hydroxide, liposomes and CpG (cytosine-phosphate-guanidine) polymers. The vaccines may also include cytokines that are known to enhance the immune response including GM-CSF, IL-2, IL-12, TNF and IFNγ.

The vaccines of the instant invention may be formulated and introduced as a vaccine through oral, intradermal, intramuscular, intraperitoneal, intravenous, subcutaneous, intranasal, and intravaginal, or any other standard route of immunization.

In formulations of the subject vaccines, wetting agents, emulsifiers and lubricants, such as sodium lauryl sulfate and magnesium stearate, as well as coloring agents, releasing agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any method well known in the art of pharmacy. The amount of composition that may be combined with a carrier material to produce a single dose may vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations, which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants, which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

In addition, vaccines may be administered parenterally as injections (intravenous, intramuscular or subcutaneous). The vaccine compositions of the present invention may optionally contain one or more adjuvants. Any suitable adjuvant can be used, such as CpG polymers, aluminum hydroxide, aluminum phosphate, plant and animal oils, and the like, with the amount of adjuvant depending on the nature of the particular adjuvant employed. In addition, the anti-infective vaccine compositions may also contain at least one stabilizer, such as carbohydrates such as sorbitol, mannitol, starch, sucrose, dextrin, and glucose, as well as proteins such as albumin or casein, and buffers such as alkali metal phosphates and the like.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers, which may be employed in the pharmaceutical compositions of the invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants. Further, the CD8⁺ T cell vaccine compositions may be encapsulated in liposomes and administered via injection.

All references cited herein are all incorporated by reference herein, in their entirety, whether specifically incorporated or not. All publications, patents, or patent applications cited herein are hereby expressly incorporated by reference for all purposes. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### EXAMPLES

### Methods

Human subjects. Peripheral blood or bone marrow for the isolation of primary CD4⁺, CD8⁺ or CD34⁺ T cells was obtained from 30 HIV-1-infected patients (Table 1) and 7 healthy adult volunteers. All patients had been on antiretroviral therapy (ART) for at least 2 years and had maintained undetectable plasma HIV-1 RNA levels (<50 copies/ml) for at least 1 year prior to study. 10 AP-treated patients were recruited from the OPTIONS cohort at the University of California San Francisco (UCSF). This study was approved by the Johns Hopkins Internal Review Board and by the UCSF Committee on Human Research. Written informed consent was provided by all study participants. HLA typing for each patient was performed by the Johns Hopkins University Immunogenetics Laboratory.
Sample preparation for deep sequencing. Peripheral blood mononuculear cells (PBMCs) were isolated from whole blood by Ficoll gradient separation. CD4⁺ T cells were purified from PBMCs by negative selection using CD4⁺ Isolation Kit II (Miltenyi). Resting CD4⁺ T cells were then purified from CD4⁺ T cells by negative selection using CD25, CD69 and HLA-DR microbeads (Miltenyi). Genomic DNA was extracted from 5 million resting CD4⁺ T cells from each patient using QlAamp DNA Mini Kit (Qiagen). The gag gene was amplified from genomic DNA by a two-round nested PCR using these primers: 5′ outer primer (5′-TTGACTAGCGGAGGCTAGAAGG-3′); 3′ outer primer (5′-GATAAAACCTCCAATTCCCCCTATC-3); 5′inner primer (5′-GAGAGATGGGTGCGAGAGCGTC-3′); 3′ inner primer (5′-CTGCTCCTGTATCTAATAGAGC-3′). For each patient, the entire genomic DNA from 5 million of resting CD4⁺ T cells was evenly distributed as template into 80 PCR reactions. The reactions were performed by using High Fidelity Plantinum Taq Polymerase (Life Technologies) following manufacturer's instruction. PCR amplicons were purified by gel extraction after gel electrophoresis.
Deep sequencing. For PacBio RS single molecule sequencing, amplicons were barcoded with a group of 10 bp indexes and then multiple samples were pooled together to generate smrtbell sequencing library following Pacific Biosciences template preparation and sequencing-C2 user guide for 2 kb insert size and using Pacific Bioscience DNA template preparation kit. For MiSeq Sequencing, the pooled amplicon DNA was end repaired, adenylated, and ligated to Illumina TruSeq adaptors and PCR enriched for 10 cycles. The resulting library was then run on bioanalyzer high sensitivity DNA chip for size and concentration determination. The library was then sequenced on MiSeq for paired end 250 bp reads. The sequence reads from PacBio and MiSeq were demultiplexed using Fastx-Toolkit.
Data analysis for deep sequencing results. For the paired MiSeq reads, the two reads were first merged using FLASH³¹. MiSeq and PacBio reads from each individual were then aligned to the reference HIV-1 consensus B Gag sequence using Bowtie2³². Custom program was written using perl scripts to identify and compute the frequency of all sequence variants that caused non-synonymous amino acid changes in each individual's relevant optimal Gag epitopes (based on reported information in the HIV Molecular

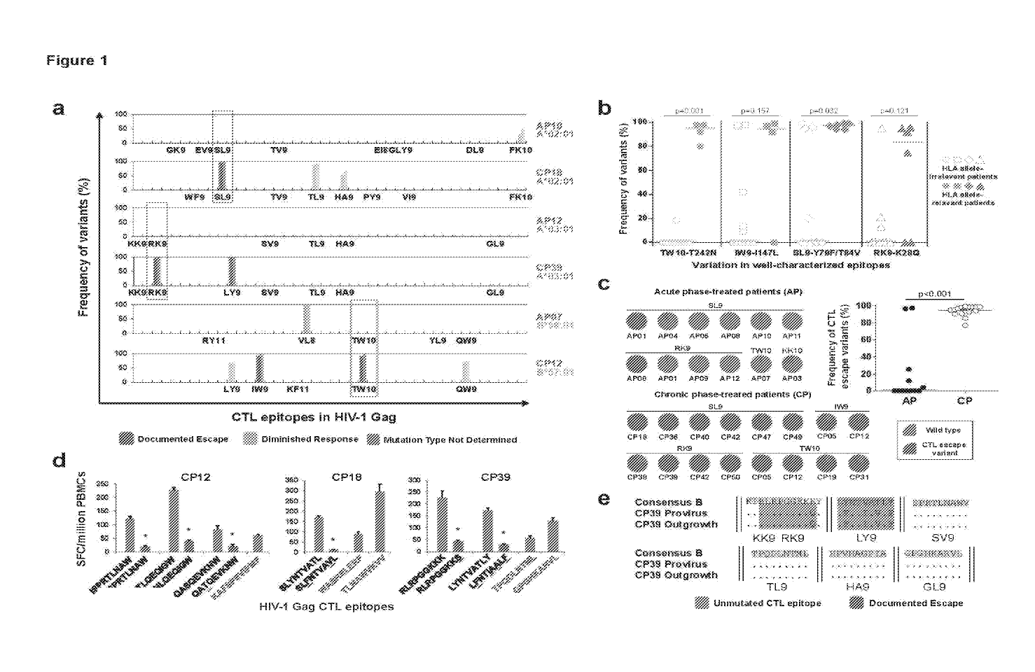

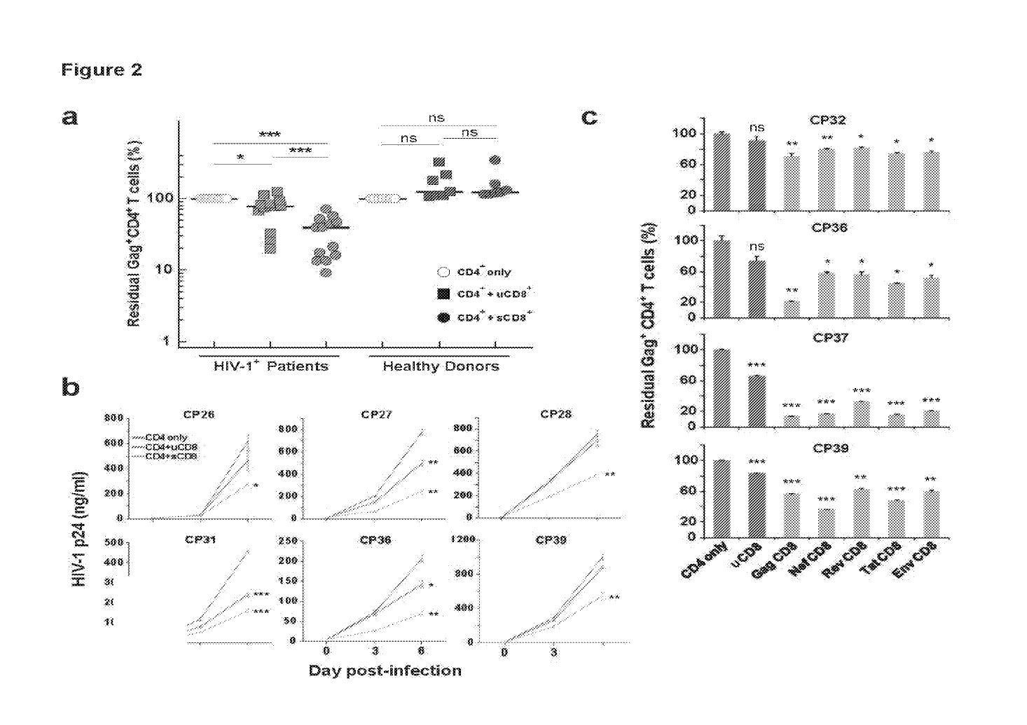

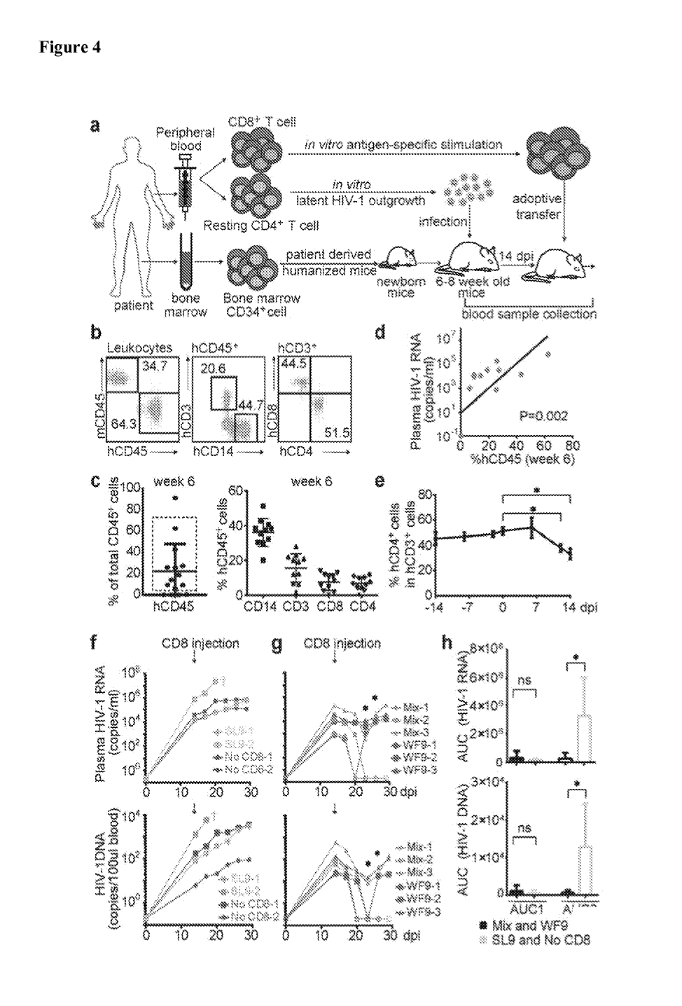

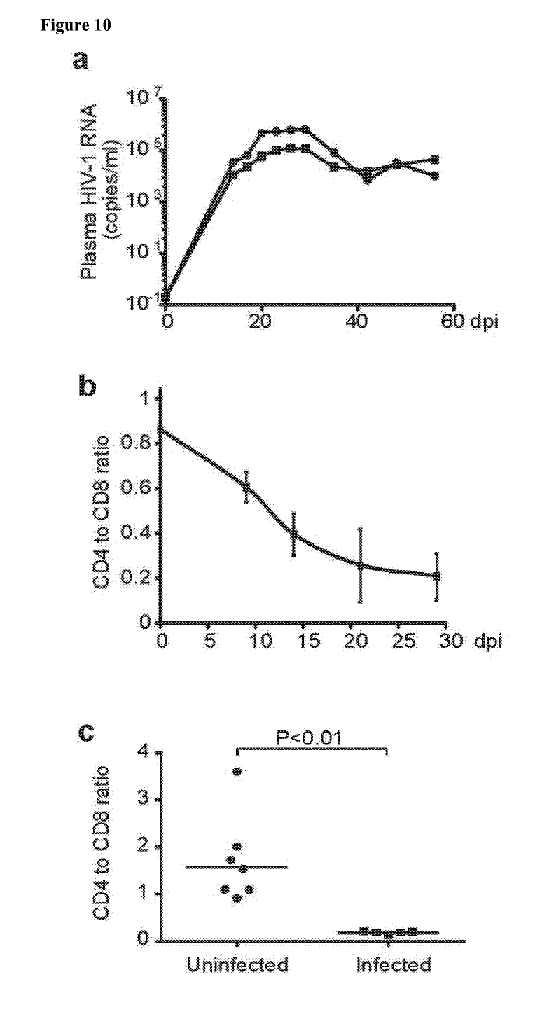

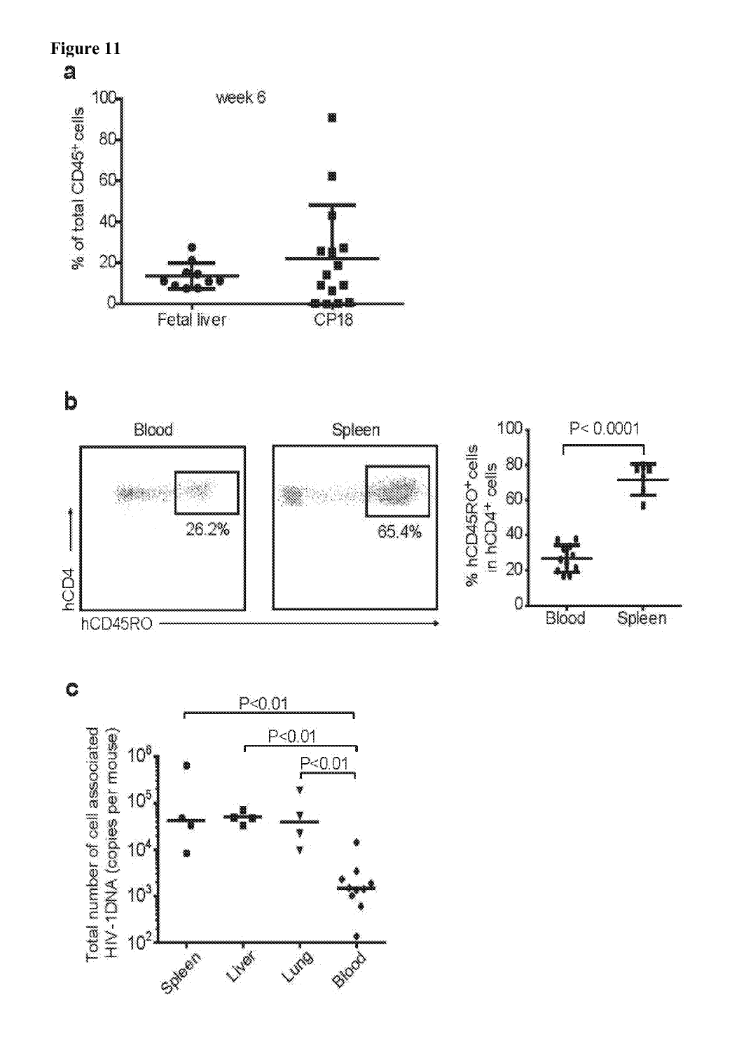

Immunology Database, Los Alamos National Laboratory, http://www.hiv.lanl.gov/content/immunology/index.html) according to their HLA type. For each individual, variants that occurred at a frequency >3% were retained. Additional for PacBio reads, sequences with identified premature stop codons were eliminated from the analyzed results. For each identified variation, the mutation type regarding CTL recognition was determined by matching with the information in the above-mentioned database. The five mutation types adopted in this paper are: Documented Escape (no CTL response when patient cells are challenged with the variant peptide), Inferred Escape (variant is predicted to be an escape mutant by longitudinal study or transmission study, but the reactivity of the variant is not tested experimentally), Diminished Response (experimental data suggest partial escape as evidenced by decreased CTL response), Susceptible Form (CTL response is elicited when patient cells are challenged with the variant peptide) and Mutation Type Not Determined (no experimental data on CTL recognition of this variant).
Elispot essays. The Elispot assays were performed using Human IFN-γ ELISpot PLUS kit (Mabtech) according to previously described³³ and manufacturer's instructions. PBMCs were added at 200,000 cells per well and synthetic peptides were added in a final concentration of 0.1, 1 or 10 μg/ml. A response is considered positive if it was 3 fold higher than the mean background (cell only control) and greater than 55 SFC/million cells. The number of specific T cells was calculated by subtracting the mean background values.
Recovery and sequencing of patient viruses from resting CD4⁺ T cells. Co-culture assays were performed to recover and amplify replication-competent viruses as previously described³⁴. The viruses were recovered from five to ten million resting CD4⁺ T cells. The concentration of outgrowth viruses was determined by p24 ELISA (PerkinElmer). Total RNA of outgrowth viruses was extracted using Trizol LS reagent (Life Technologies). Residual DNA was then removed by TURBO DNase (Life Technologies) treatment. First strand cDNA was synthesized using SuperScript III Reverse Transcriptase (Life Technologies) and the gag gene was amplified from cDNA using the gag outer primer pair mentioned above. The PCR amplicons were then purified by gel extraction and sequenced by regular Sanger sequencing.
In vitro HIV-1 infection. PBMCs from HIV-1-infected patients and healthy donors were stimulated by adding 0.5 μg/ml Phytohaemagglutinin (PHA) and IL-2 (100 U/ml) to basal media (RPMI with 10% heat-inactivated fetal bovine serum and antibiotics) for three days prior to isolation of CD4⁺ T cells. Each patient's activated CD4⁺ T cells were infected with viruses recovered from the same patient's resting CD4⁺ T cells. Healthy donor's CD4⁺ cells were infected with a lab strain virus, BaL. Virus concentration used in infection was equivalent to the p24 concentration of 200 ng/ml. All infections were performed by centrifugation of target cells with virus at 1,200 g for 2 hours.
Stimulation of CD8⁺ T cells. PBMCs from CP-treated patients were cultured in the presence of IL-2 (100 U/ml) with a mixture of consensus B Gag (or Nef, Rev, Tat, Env) peptides (800 ng/ml for each) (NIH AIDS Reagent Program), or with individual synthetic peptide (0.5 μ/ml) (Genemed Synthesis). CD8⁺ T cells were purified after six days of incubation by positive selection using human CD8 microbeads (Miltenyi). To monitor CTL proliferation, PBMCs were stained with CFSE (Life Technologies) prior to incubation and with relevant pentamer (Proimmune) after incubation. PBMCs were then stained with CD8-APC (Becton Dickson, BD) and analyzed by flow cytometry using FACS Canto II (BD).
Co-culture of autologous CD4⁺ and CD8⁺ T cells. Three hours after infection, CD4⁺ T cells were mixed with autologous unstimulated or stimulated CD8⁺ T cells at 1:1 ratio in basal media at 5 million cells per ml. Two days after co-culture, enfuvirtide (T-20, Roche) was added into the culture at 10 μM to prevent further infection events except if the measurement was p24 ELISA. Three days after co-culture, cells were stained with CD8-APC (BD) first, fixed and permeabilized with Cytoperm/Cytofix (BD pharmingen), then stained for intracellular p24 Gag (PE, Coulter). Cells were analyzed by flow cytometry using FACS Canto II (BD). For measurement of viral growth, 5 μl of supernatant was taken from the co-culture at Days 0, 3 and 6, and subjected to p24 ELISA. For analysis of cell contact dependence, CD4⁺ and CD8⁺ T cells were placed in separate chambers of trans-well plates (0.4 μm, Costar).
Generation and infection of patient-derived humanized mice. The previously reported MISTRG mouse in the Rag2^{-/-} Il2rg^{-/-} 129×Balb/c (N2) genetic background harbors knock-in replacement of the endogenous mouse Csf1, Csf2, Il3 and Tpo genes with humanized version and a BAC transgene encoding human SIRPα²⁵. We generate Sirpa^{(KI)} mouse which harbors a knock-in replacement of the endogenous mouse Sirpa gene with a humanized version. Sirpa^{(KI)} mouse will be thoroughly described elsewhere. The improved MIS^{(KI)}TRG mouse was generated by breeding Sirpa^{(KI)} mice to MITRG mice. All animal experimentations were performed in compliance with Yale Institutional Animal Care and Use Committee protocols. MIS^{(KI)}TRG mice were maintained with continuous treatment with enrofloxacin in the drinking water (Baytril, 0.27 mg/ml). Patient bone marrow or fetal liver CD34⁺ cells were isolated by CD34 microbeads selection (miltenyi). Newborn mice (within first 3 days of life) were sublethally irradiated (X-ray irradiation with X-RAD 320 irradiator, PXi; 1×150 cGy) and 100,000 fetal liver or 250,000 patient CD34⁺ cells in 20 μl of PBS were injected into the liver with a 22-gauge needle (Hamilton Company). 6-8 weeks after engraftment, mice engrafted with patient CD34⁺ cells were infected by i.v. with HIV-1 (100 ng p24) which was recovered and expanded from the resting CD4⁺ T cells of the same patient (CD34⁺ cell donor), as mentioned above. Mice engrafted with fetal liver CD34⁺ cells were infected by i.v. with HIV-1 BaL (100 ng p24). Mice with less than 5% human CD45⁺ cells in the peripheral blood were excluded from the infection study. Mice with more than 70% human CD45 ⁺ cells in the peripheral blood were also excluded because they were unhealthy due to human macrophage/monocytes caused anemia³³. 20 million autologous CD8⁺ T cells with or without pre-stimulation were injected by i.v. 9 or 14 days after infection. Group allocation was blinded. Peripheral blood samples were collected by retro-orbital bleeding at different time points before and after injection of CD8⁺ T cells. Engraftment of human CD45⁺ cells as well as lymphoid and myeloid subsets was determined by flow cytometry. Plasma HIV-1 RNA in peripheral blood was measured by one-step reverse transcriptase (Invitrogen) real-time PCR using the following primers and probe, described previously⁸: forward (5′→3′) ACATCAAGCAGCCATGCAAAT, reverse (5′→3′) TCTGGCCTGGTGCAATAGG and probe (5′→3′) VIC-CTATCCCATTCTGCAGCTTCCTCATTGATG-TAMRA. Assay sensitivity is 200 RNA copies per ml of plasma. HIV-1 DNA in peripheral blood was also measured by real time PCR using the same primers and probe mentioned above, with assay sensitivity at 5 copies per 100 μl of blood. Total viral DNA in PBMCs was determined by measuring copies of viral DNA/100 μl blood and blood volume per mouse (80 μl blood/1 g body weight). To quantitate total viral DNA in tissues, spleens, livers and lungs of infected mice were collected. For the spleen, single-cell suspensions were treated with ACK lysis buffer. Liver and lung leukocytes were isolated by digesting tissues with 100 U/ml collagenase IV and 0.02 mg/ml DNase I (Sigma), followed by density gradient centrifugation.
Statistical analysis. For comparison of HIV-1 variant frequency (FIGS. 1b and c) and viral infection in HIV-1 BaL-infected mice (FIGS. 10 and 11), we applied Mann-Whitney tests. For comparison of inhibitory effect of autologous CTLs (FIG. 2a), we applied a Wilcoxon matched pairs test. For comparison of viral replication in humanized mice (FIGS. 4f, g and h), we applied an unpaired t test. For all other comparisons, paired t tests were applied. All tests were calculated by the GraphPad Prism 6 software, and conducted as two-tailed tests with a type I error rate of 5%.

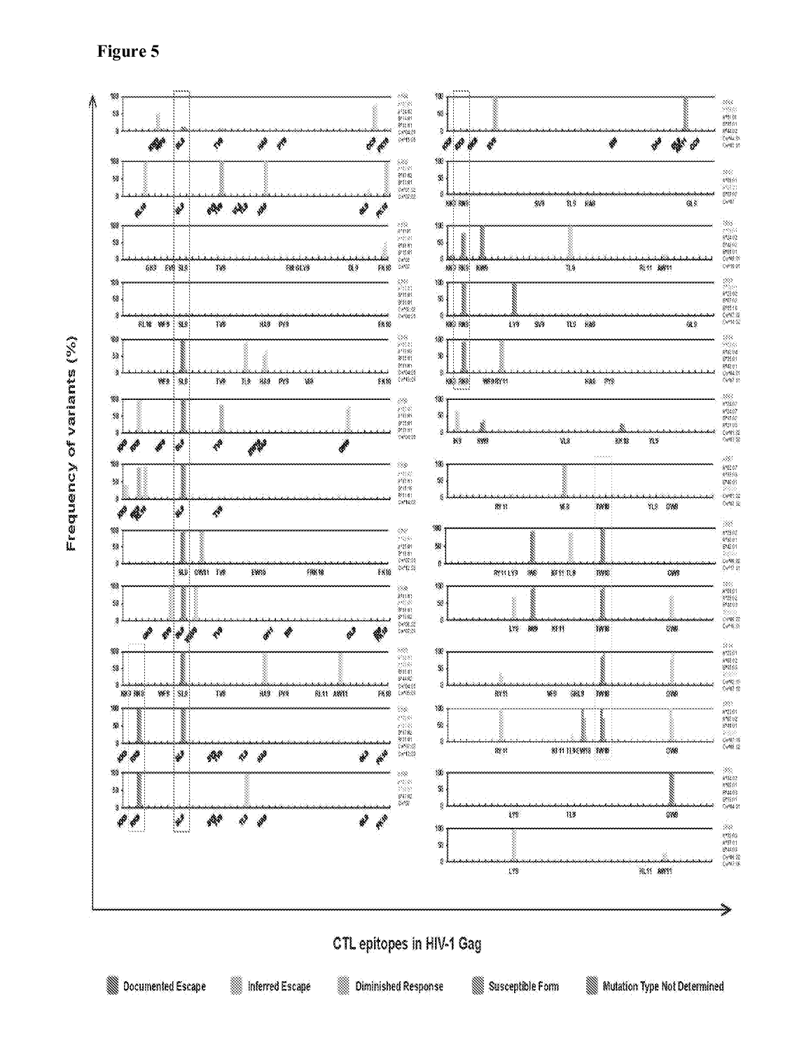

To investigate CTL escape variants in the latent reservoir, the proviral HIV-1 DNA in resting CD4⁺ T cells from 25 patients was deep sequenced (Table 2). Among them, 10 initiated ART during the acute phase (AP, within 3 months of infection) while the other 15 initiated ART during the chronic phase (CP) of infection. The sequencing was focused on Gag because it is an important target of the CTL response²³ and is highly conserved, which facilitates detection of escape variants. Prior data from our lab showed that previously documented CTL escape variants completely dominated the viral reservoirs of nearly all CP-treated patients (FIG. 5 and Table 2). This trend is especially obvious for several well characterized CTL epitopes: the HLA-A2-restricted epitope SLYNTVATL (SL9), the HLA-A3-restricted epitope RLRPGGKKK (RK9) and the HLA-B57/58-restricted epitope TSTLQEQIGW (TW10) (FIG. 1a and FIG. 5). In these epitopes, close to 100% of the sequences harbored escape mutations. Comparison of mutation frequencies between HLA allele-relevant and -irrelevant epitopes in CP-treated patients suggests these CTL escape mutations identified are specific to patient's HLA type (FIG. 1b). By contrast, except for SL9 from AP01 and RK9 from AP08, few if any CTL escape mutations were archived in AP-treated patients (FIG. 1c and FIG. 5). The striking difference between AP- and CP-treated patients (FIG. 1c) indicates that unless treatment is initiated within the first several months of infection, the latent reservoir becomes almost completely dominated by variants resistant to dominant CTL responses.

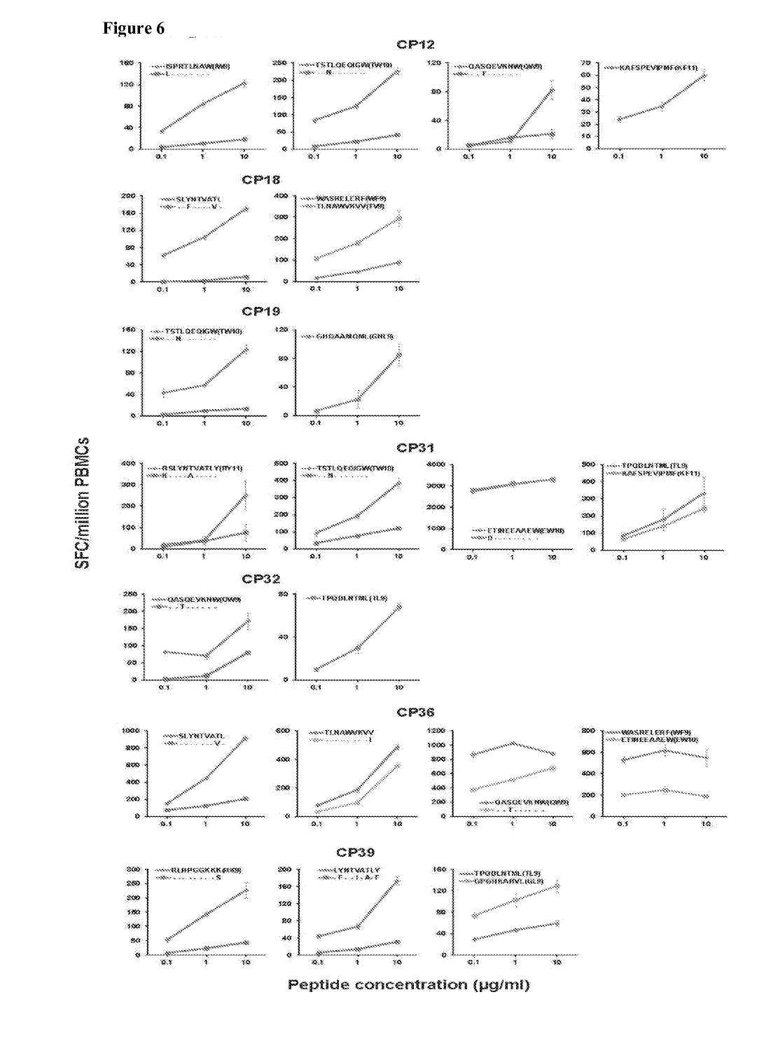

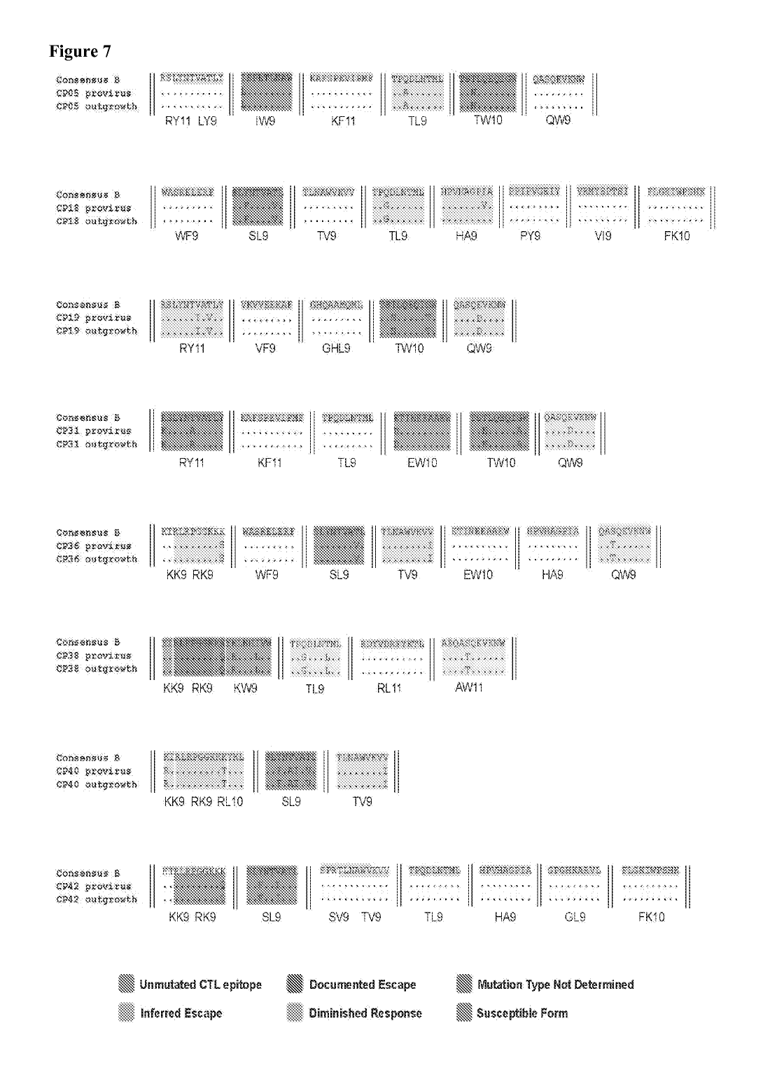

To confirm variants detected at high frequency in the latent reservoir represent functional CTL escape mutants, cells from 7 CP-treated subjects were tested for reactivity to synthetic peptides representing wild-type and mutant versions of the relevant epitopes. As expected, there were only minimal responses to previously documented CTL escape mutants by patient CD8⁺ T cells, and no de novo response was detected (FIG. 1d and FIG. 6). In contrast, all tested subjects retained a strong response to peptides representing the wild-type epitopes, suggesting wild-type virus was initially transmitted, with subsequent evolution of CTL escape variants. Most HIV-1 proviruses detected in patients are defective²⁴. Therefore, to determine whether these CTL escape variants can be reactivated and lead to viral rebound if therapy is stopped, replication-competent viruses were isolated from the latent reservoirs of 9 CP-treated patients. It was found that all dominant CTL escape mutations identified in proviruses in resting CD4⁺ T cells were also present in the replication-competent viruses that grew out after T cell activation (FIG. 1e and FIG. 7), indicating that these CTL escape variants not only dominate the population of proviruses, but can also be released and replicate once latency is reversed.

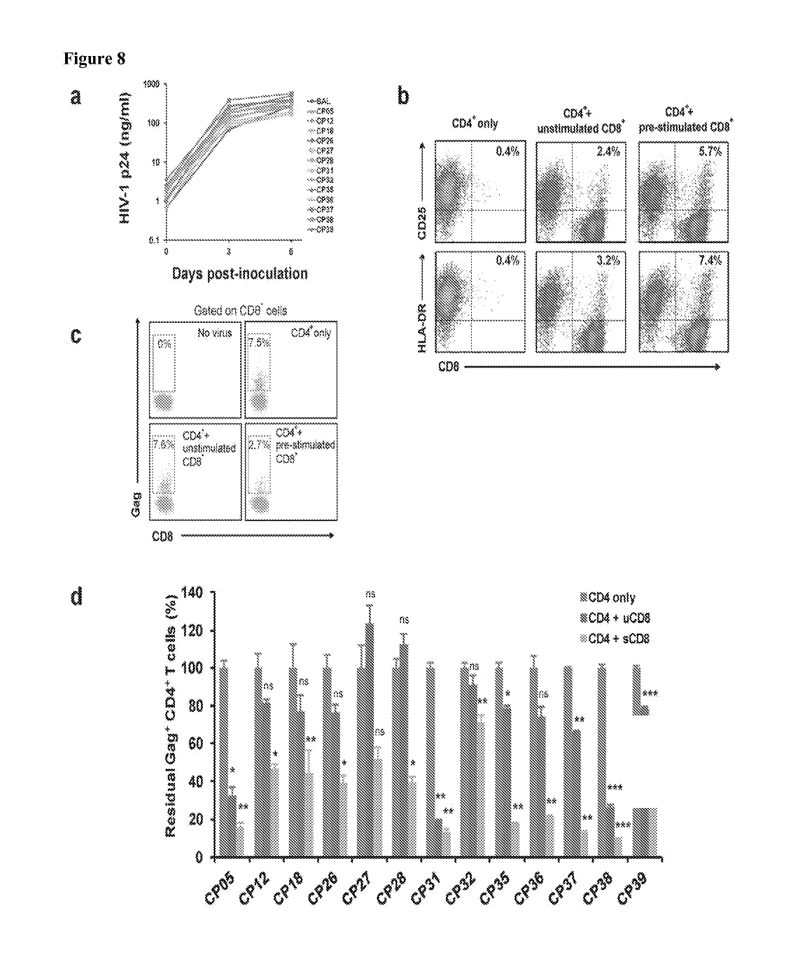

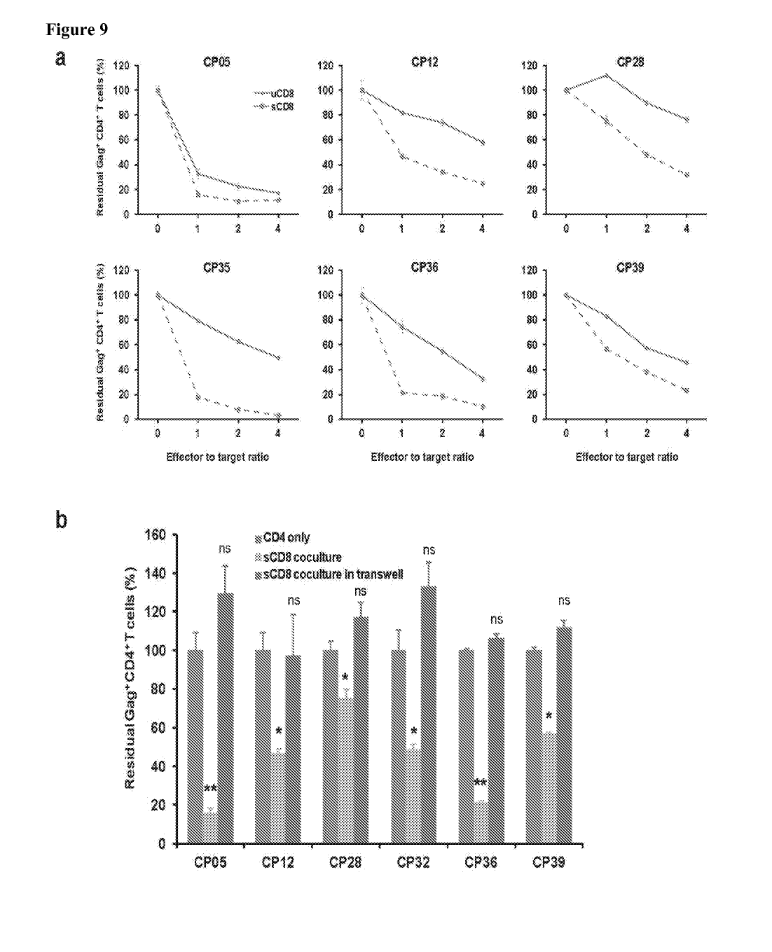

Whether the host CTL response could recognize and eliminate the cells infected with these escape variants were investigated next. A ctivated CD4⁺ T cells from these patients were infected with autologous, replication-competent virus derived from the latent reservoir (FIG. 8a). The infected cells were then co-cultured with autologous CD8⁺ T cells, either unstimulated or pre-stimulated, to assess HIV-1-specific cytolytic activity. Non-specific activation of CD8⁺ T cells was not observed after co-cultured with PHA-activated CD4⁺ T cells (FIG. 8b). From all 13 CP-treated subjects tested, CD8⁺ T cells pre-stimulated by a Gag peptide mixture efficiently killed autologous infected CD4⁺ T cells (median 61% elimination), while unstimulated CD8⁺ T cells from most subjects had significantly less effect (median 23% elimination) (FIG. 2a and FIGS. 8c and 8d). CD8⁺ T cells from 7/7 healthy donors completely failed to eliminate autologous infected cells (FIG. 2a), confirming the observed killing was HIV-1-specific. The killing effect was enhanced by increasing the effector to target (E:T) ratio (FIG. 9a), and was cell-cell contact dependent (FIG. 9b). When the co-culture was maintained over time in the absence of ART, viral replication was significantly reduced, but not completely inhibited by pre-stimulated CD8⁺ T cells (FIG. 2b). Itwas found that peptide mixtures from other HIV-1 proteins (Nef, Tat, Rev, and Env) could also boost CTL responses and facilitate the elimination of infected cells (FIG. 2c) and that CTLs pre-stimulated by Gag peptides generally had the highest activity. Together, these results demonstrate that chronically infected patients retain CTL clones that can recognize and eliminate autologous infected CD4⁺ T cells, despite the presence of CTL escape mutations in dominant epitopes. However, these clones require stimulation with antigen for optimal activity.

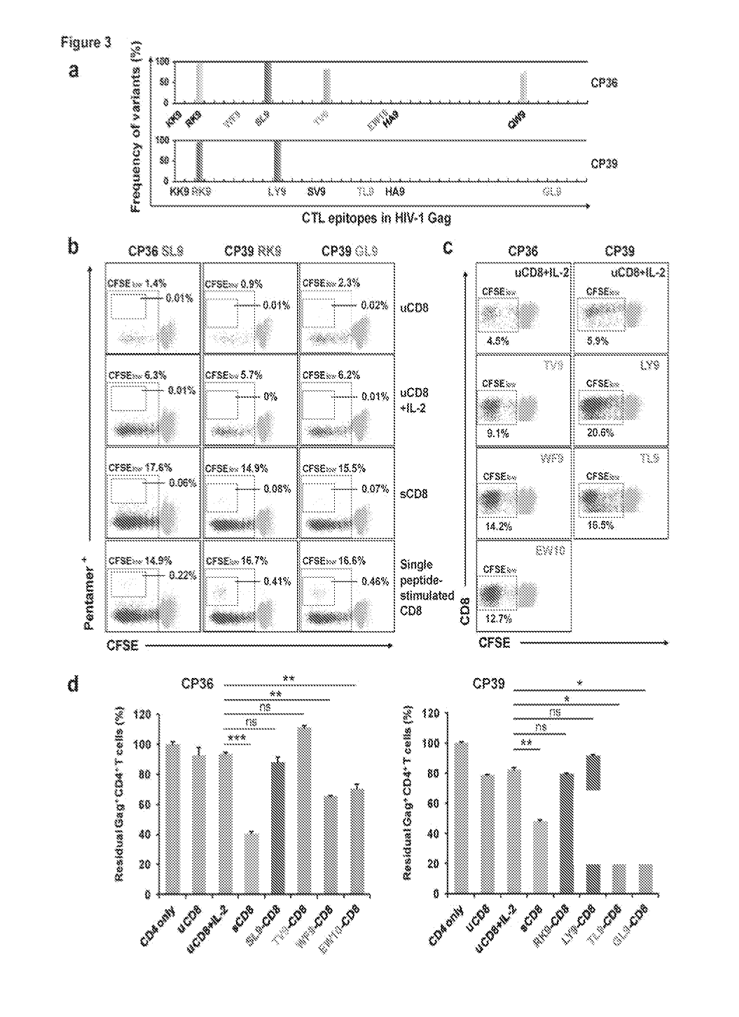

To further characterize which CTL population contributed to the elimination of cells infected by CTL escape variants, the killing activity of two specific CTL populations were compared: the one that targets epitopes in which escape has been identified and the one that targets unmutated epitopes (FIG. 3a). CD8⁺ T cells from CP36 and CP39 were pre-stimulated with IL-2 and different synthetic peptides representing the wild-type forms of the relevant epitopes. After incubation for 6 days, each CTL population exhibited significant proliferation compared to no treatment or IL-2 alone (FIGS. 3b and c). Pentamer staining for three available epitopes revealed that the number of epitope-specific CD8⁺ T cells increased dramatically after stimulation with wild-type peptides (FIG. 3b). After co-culture with autologous target cells infected with latent reservoir-derived viruses, CTLs targeting unmutated epitopes clearly showed stronger cytolytic activity than the IL-2 only controls, while CTLs targeting epitopes with identified escaped mutations showed no significant killing (FIG. 3d). CTLs pre-stimulated by the Gag peptide mixture exhibited stronger killing than all single peptide-stimulated populations (FIG. 3d).

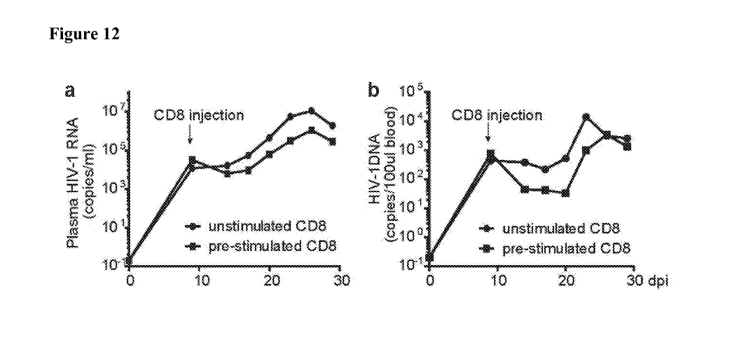

To test whether CTL that recognize unmutated viral epitopes can inhibit HIV-1 replication and clear infected cells in vivo, patient-derived humanized mice using an improved version of a recently reported mouse system named MISTRG²⁵ were generated. Whereas the previously reported MISTRG mice bear a BAC transgene encoding human SIRPα, the newly generated MIS^{(KI)}TRG mice harbor a knock-in replacement of the endogenous mouse Sirpa gene with a humanized version. With humanization by knock-in replacement of the Csf1, Csf2, Il3, Tpo and Sirpa genes in the Rag2^{-/-} Il2rg^{-/-} genetic background, MIS^{(KI)}TRG mice are highly permissive for human hematopoiesis and support the reconstitution of robust human lymphoid and myelomonocytic systems. With the demonstrated development of functional T-lymphocytes and monocytes/macrophages, MIS^{(KI)}TRG mice provide a useful humanized mouse host for HIV-1 infection studies. Bone marrow biopsies were obtained from study participants and purified CD34⁺ cells were used to reconstitute the MIS^{(KI)}TRG mice. These patient-derived humanized mice were infected with primary HIV-1 isolates grown from resting CD4⁺ T cells of the same patient and then evaluated antiviral effect of autologous CD8⁺ T cells (FIG. 4a). MIS^{(KI)}TRG mice engrafted with CP18 bone marrow CD34⁺ cells successfully developed human T-lymphocyte and monocyte/macrophage subsets (FIGS. 4b and c), which were sufficient to support HIV-1 infection (FIG. 4d). Plasma HIV-1 RNA levels peaked 20-30 days after infection (FIG. 10a). Depletion of CD4⁺ T cells was clearly evident 12 days after infection in peripheral blood and spleen (FIG. 4e and FIGS. 10b and 10c). Cell-associated HIV-1 RNA was detected in both T cells and macrophages/monocytes (FIG. 10d). Viral infection was also observed in various tissues where a large number of memory CD4⁺ T cells were detected (FIG. 11). In control mice or mice that received autologous patient CD8⁺ T cells pre-stimulated with a peptide representing the unmutated dominant SL9 epitope, levels of plasma HIV-1 RNA and proviral DNA in peripheral blood continued to increase from day 14 to day 29 after infection (FIG. 40. In sharp contrast, mice that received CD8⁺ T cells pre-stimulated with unmutated epitopes (Gag mix or WF9) had a significantly lower level of viral replication (FIG. 4g and h). Dramatic decreases in plasma HIV-1 RNA of 100- to 1,000-fold were observed in all three mice that received CD8⁺ T cells pre-stimulated with the mixture of Gag peptides including dominant and subdominant epitopes. Two of three mice had undetectable levels of plasma HIV-1 RNA and proviral DNA in peripheral blood measured at three time points (FIG. 4g). The same experiments were performed using patient CP36-derived humanized mice and reduction of peripheral HIV-1 RNA and DNA levels was also observed in mice that received CP36 CD8⁺ T cells pre-stimulated with the mixture of Gag peptides (FIG. 12). Since the post-engraftment lifespan of MIS^{(KI)}TRG mice is only 10-12 weeks³³, the acute phase of HIV-linfection and demonstrate the in vivo functionality of patient CD8⁺ T cells was only investigated. Future developments of the MIS^{(KI)}TRG model will prolong the post-engraftment lifespan of the mice and will allow the studies of establishment and clearance of HIV-1 latent reservoir in vivo. Together, these in vitro and in vivo experiments demonstrate that only the CTL clones targeting unmutated epitopes are effective against cells infected with the viral variants that are likely to represent the major source of rebound HIV-1 after reversal of latency.

The seeding of the HIV-1 latent reservoir starts just a few days after infection²⁶, prior to the development of robust CTL response¹⁴. This is consistent with the finding that patients who initiated treatment early in acute infection have few if any CTL escape variants archived in the latent reservoir. However, if treatment was initiated in chronic infection, CTL escape variants became dominant in the latent reservoir, indicating a complete replacement of the initially established ‘wild-type’ reservoir. The mechanism behind this replacement warrants further investigation, but likely reflects the dynamic nature of the reservoir in untreated infection. In any event, the overwhelming presence of escape variants in the latent reservoir of chronic patients certainly presents an additional barrier to eradication efforts. The striking difference between AP- and CP-treated patients presents another argument for early treatment of HIV-1 infection; early treatment not only reduces the size of the latent reservoir²⁷, but also alters the composition of the reservoir, as shown here, in a way that may enhance the efficacy of potential CTL-based eradication therapies.

The hierarchy of HIV-1-specific CTL response in acute infection appears to play a significant role in initial viral suppression as demonstrated by the fact that certain immunodominant CTL populations are frequently linked to lower set point viremia later in infection^{17,28}. These immunodominant responses in acute infection have been identified as the major selection force driving the development of CTL escape mutations^{13, 20}. Here it was shown that these immunodominant response-driven mutations are not only archived in the latent reservoir, but also in fact dominate the latent provirus population in CP-treated patients. Therefore, directing CTL responses to unmutated viral epitopes is essential to clear latent HIV-1. Due to bias in antigen presentation or recognition²⁹, common vaccination strategies will likely re-stimulate immunodominant CTL clones which do not kill infected cells after reversal of latency. Stimulation of CTL responses with viral peptides circumvents antigen processing and is able to elicit broad-spectrum CTL responses against unmutated regions of viral proteins. These study suggests that latent HIV-1 can be eliminated in chronically infected patients despite the overwhelming presence of CTL escape variants. Future directions in therapeutic vaccine design need to focus on boosting broad CTL responses as also reported elsewhere³⁰ and/or manipulating immuno dominance.

### REFERENCES

1. Siliciano, J. D. et al. Long-term follow-up studies confirm the stability of the latent reservoir for HIV-1 in resting CD4+ T cells. Nat Med 9, 727-8 (2003).
2. Strain, M. C. et al. Heterogeneous clearance rates of long-lived lymphocytes infected with HIV: intrinsic stability predicts lifelong persistence. Proc Natl Acad Sci USA 100, 4819-24 (2003).
3. Finzi, D. et al. Identification of a reservoir for HIV-1 in patients on highly active antiretroviral therapy. Science 278, 1295-300 (1997).
4. Chun, T. W. et al. Quantification of latent tissue reservoirs and total body viral load in HIV-1 infection. Nature 387, 183-8 (1997).
5. Richman, D. D. et al. The challenge of finding a cure for HIV infection. Science 323, 1304-7 (2009).
6. Archin, N. M. et al. Expression of latent HIV induced by the potent HDAC inhibitor suberoylanilide hydroxamic acid. AIDS Res Hum Retroviruses 25, 207-12 (2009).
7. Contreras, X. et al. Suberoylanilide hydroxamic acid reactivates HIV from latently infected cells. J Biol Chem 284, 6782-9 (2009).
8. Archin, N. M. et al. Administration of vorinostat disrupts HIV-1 latency in patients on antiretroviral therapy. Nature 487, 482-5 (2012).
9. Shan, L. et al. Stimulation of HIV-1-specific cytolytic T lymphocytes facilitates elimination of latent viral reservoir after virus reactivation. Immunity 36, 491-501 (2012).
10. Walker, B. D. et al. HIV-specific cytotoxic T lymphocytes in seropositive individuals. Nature 328, 345-8 (1987).
11. Koup, R. A. et al. Temporal association of cellular immune responses with the initial control of viremia in primary human immunodeficiency virus type 1 syndrome. J Virol 68, 4650-5 (1994).
12. Borrow, P. et al. Antiviral pressure exerted by HIV-1-specific cytotoxic T lymphocytes (CTLs) during primary infection demonstrated by rapid selection of CTL escape virus. Nat Med 3, 205-11 (1997).
13. Goonetilleke, N. et al. The first T cell response to transmitted/founder virus contributes to the control of acute viremia in HIV-1 infection. J Exp Med 206, 1253-72 (2009).
14. McMichael, A. J., Borrow, P., Tomaras, G. D., Goonetilleke, N. & Haynes, B. F. The immune response during acute HIV-1 infection: clues for vaccine development. Nat Rev Immunol 10, 11-23 (2010).
15. Phillips, R. E. et al. Human immunodeficiency virus genetic variation that can escape cytotoxic T cell recognition. Nature 354, 453-9 (1991).
16. Koup, R. A. Virus escape from CTL recognition. J Exp Med 180, 779-82 (1994).
17. Goulder, P. J. et al. Late escape from an immunodominant cytotoxic T-lymphocyte response associated with progression to AIDS. Nat Med 3, 212-7 (1997).
18. Goulder, P. J. & Watkins, D. I. HIV and SIV CTL escape: implications for vaccine design. Nat Rev Immunol 4, 630-40 (2004).
19. Henn, M. R. et al. Whole genome deep sequencing of HIV-1 reveals the impact of early minor variants upon immune recognition during acute infection. PLoS Pathog 8, e1002529 (2012).
20. Liu, M. K. et al. Vertical T cell immunodominance and epitope entropy determine HIV-1 escape. J Clin Invest 123, 380-93 (2013).
21. Rolland, M., Nickle, D. C. & Mullins, J. I. HIV-1 group M conserved elements vaccine. PLoS Pathog 3, e157 (2007).
22. Letourneau, S. et al. Design and pre-clinical evaluation of a universal HIV-1 vaccine. PLoS One 2, e984 (2007).
23. Yu, X. G. et al. Consistent patterns in the development and immunodominance of human immunodeficiency virus type 1 (HIV-1)-specific CD8+ T-cell responses following acute HIV-1 infection. J Virol 76, 8690-701 (2002).
24. Ho, Y. C. et al. Replication-Competent Noninduced Proviruses in the Latent Reservoir Increase Barrier to HIV-1 Cure. Cell 155, 540-51 (2013).
25. Rongvaux, A. et al. Development and function of human innate immune cells in a humanized mouse model. Nat Biotechnol 32, 364-72 (2014).
26. Whitney, J. B. et al. Rapid seeding of the viral reservoir prior to SIV viraemia in rhesus monkeys. Nature, 74-77 (2014).
27. Ananworanich, J. et al. Impact of multi-targeted antiretroviral treatment on gut T cell depletion and HIV reservoir seeding during acute HIV infection. PLoS One 7, e33948 (2012).
28. Goulder, P. J. et al. Evolution and transmission of stable CTL escape mutations in HIV infection. Nature 412, 334-8 (2001).
29. Le Gall, S., Stamegna, P. & Walker, B. D. Portable flanking sequences modulate CTL epitope processing. J Clin Invest 117, 3563-75 (2007).
30. Hansen, S. G. et al. Cytomegalovirus vectors violate CD8+ T cell epitope recognition paradigms. Science 340, 1237874 (2013).
31. Magoc, T. & Salzberg, S. L. FLASH: fast length adjustment of short reads to improve genome assemblies. Bioinformatics 27, 2957-63 (2011).
32. Langmead, B. & Salzberg, S. L. Fast gapped-read alignment with Bowtie 2. Nat Methods 9, 357-9 (2012).
33. Streeck, H., Frahm, N. & Walker, B. D. The role of IFN-gamma Elispot assay in HIV vaccine research. Nat Protoc 4, 461-9 (2009).
34. Siliciano, J. D. & Siliciano, R. F. Enhanced culture assay for detection and quantitation of latently infected, resting CD4+ T-cells carrying replication-competent virus in HIV-1-infected individuals. Methods Mol Biol 304, 3-15 (2005).

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

TABLE 1
Peripheral blood or bone marrow for the isolation of primary
CD4+, CD8+ or CD34+ T cells was obtained from
30 HIV-1-infected patients and 7 healthy adult volunteers.

| | | | Plasma | | Treatment |
|---|---|---|---|---|---|
| | | CD4 | HIV-1 | Time on | start time |
| | Year of | count* | RNA^{†} | ART | after infection |
| Patient | Diagnosis | (cells/μl) | (copies/ml) | (years) | (days) |
| AP01 | 2006 | 1251 | <50 | 7 | 64 |
| AP03 | 2004 | 595 | <50 | 9 | 34 |
| AP04 | 1998 | 953 | <50 | 15 | 77 |
| AP05 | 2002 | 618 | <50 | 11 | 39 |
| AP07 | 2012 | 592 | <50 | 1.5 | 67 |
| AP08 | 2008 | 780 | <50 | 6 | 28 |
| AP09 | 2012 | 1069 | <50 | 2 | 39 |
| AP10 | 2006 | 513 | <50 | 7 | 50 |
| AP11 | 2007 | 874 | <50 | 6 | 10 |
| AP12 | 2007 | 629 | <50 | 6 | 15 |
| CP05 | 2001 | 500 | <50 | 10 | >180 |
| CP12 | 1997 | 1074 | <50 | 15 | >180 |
| CP18 | 1998 | 773 | <50 | >4 | >180 |
| CP19 | 2006 | 620 | <50 | 6 | >180 |
| CP26 | 1994 | 640 | <50 | 16 | >180 |
| CP27 | 1987 | 784 | <50 | 4 | >180 |
| CP28 | 1998 | 614 | <50 | 6 | >180 |
| CP31 | 2000 | 619 | <50 | 10 | >180 |
| CP32 | 1999 | 780 | <50 | 2 | >180 |
| CP35 | 2002 | 738 | <50 | 10 | >180 |
| CP36 | 2003 | 1119 | <50 | 4 | >180 |
| CP37 | 1999 | 730 | <50 | 12 | >180 |
| CP38 | 1986 | 870 | <50 | 3 | >180 |
| CP39 | 1996 | 1552 | <50 | 10 | >180 |
| CP40 | 2002 | 544 | <50 | 7 | >180 |
| CP42 | 1987 | 684 | <50 | 16 | >180 |
| CP47 | 1986 | 792 | <50 | 14 | >180 |
| CP48 | 1998 | 641 | <50 | 8 | >180 |
| CP49 | 1992 | 864 | <50 | 5 | >180 |
| CP50 | 2001 | 964 | <50 | 4 | >180 |

TABLE 2
Deep sequencing results for viral variants archived in the latent reservoir.

| | | | | | Variation | Variation | |
|---|---|---|---|---|---|---|---|
| | | Epitope | | | Frequency | Frequency | Mutation |
| Patient | HLA Type | Name | Epitope¹ | Variant² | (%) (PacBio) | (%) (MiSeq) | Type³ |
| CP05 | A*29:02 | RY11 | RSLYNTVATLY (A30) | — | 0 | 0 | — |
| | A*30:01 | LY9 | LYNTVATLY (A29:02) | — | 0 | 0 | — |
| | B*42:01 | IW9 | ISPRTLNAW (B57:01) | I147L | 89.4 | 92.3 | E |
| | B*57:01 | KF11 | KAFSPEVIPMF (B57:01) | — | 0 | 0 | — |
| | Cw*06:02 | TL9 | TPQDLNTML (B42:01) | Q182A | 86.6 | 88.1 | MTND |
| | Cw*17:01 | TW10 | TSTLQEQIGW (B57:01) | T242N | 98.4 | 98.7 | E |
| | | QW9 | QASQEVKNW (B57:01) | — | 0 | 0 | — |
| CP12 | A*01:01 | LY9 | LYNTVATLY (A29:02, B44:03) | T81A, V82I | 67.1 | 68.6 | MTND |
| | A*29:02 | IW9 | ISPRTLNAW (B57:01) | I147L | 93 | 97.3 | E |
| | B*44:03 | KF11 | KAFSPEVIPMF (B57:01) | — | 0 | 0 | — |
| | B*57:01 | TW10 | TSTLQEQIGW (B57:01) | T242N | 91.3 | 99.1 | B |
| | Cw*06:02 | QW9 | QASQEVKNW (B57:01) | S310T | 69.8 | 72.8 | MTND |
| | Cw*16:01 | | | | | | |
| CP18 | A*02:01 | WF9 | WASRELERF (B35:01) | — | 0 | 0 | — |
| | A*33:03 | SL9 | SLYNTVATL (A02:01) | Y79F, V82I, | 97.4 | 98.8 | E |
| | B*35:01 | | | T84V | | | |
| | B*81:01 | TV9 | TLNAWVKVV (A02:01) | — | 0 | 0 | — |
| | Cw*04:01 | TL9 | TPQDLNTML (B81:01) | Q182G/A/V | 87.6 | 93.4 | MTND |
| | Cw*18:01 | HA9 | HPVHAGPIA (B35:01) | I223V | 53.7 | 66.6 | MTND |
| | | PY9 | PPIPVGEIY (B35:01) | — | 0 | 0 | — |
| | | FK10 | FLGKIWPSHK (A0201) | — | 0 | 0 | — |
| CP19 | A*23:01 | RY11 | RSLYNTVATLY (B58) | V82I, T84V | 35.9 | 24.6 | MTND |
| | A*68:02 | VF9 | VKVVEEKAF (B15:03) | — | 0 | 0 | — |
| | B*15:03 | GHL9 | GHQAAMQML (B15) | — | 0 | 0 | — |
| | B*58:01 | TW10 | TSTLQEQIGW (B58:01) | T242N, G248T | 82.9 | 98.3 | E |
| | Cw*02:10 | QW9 | QASQEVKNW (B58:01) | E312D | 78.1 | 97.9 | MTND |
| | Cw*07:18 | | | | | | |
| CP31 | A*23:01 | RY11 | RSLYNTVATLY (B58) | R76K, T81A | 99.5 | 92.3 | E |
| | A*66:02 | KF11 | KAFSPEVIPMF (B58:01) | — | 0 | 0 | — |
| | B*41:01 | TL9 | TPQDLNTML (C08:02) | Q182G, T186L | 0 | 22.9 | MTND |
| | B*58:01 | EW10 | ETINEEAAEW (B58) | E203D | 95.1 | 69.2 | SF |
| | Cw*07:18 | TW10 | TSTLQEQIGW (B58:01) | T242N, G248A | 99.1 | 71.7 | E |
| | Cw*08:02 | QW9 | QASQEVKNW (B58:01) | E312D | 99 | 70.4 | MTND |
| CP32 | A*34:02 | LY9 | LYNTVATLY (B44:03) | — | 0 | 0 | — |
| | A*68:01 | TL9 | TPQDLNTML (B53:01) | — | 0 | 0 | — |
| | B*44:03 | QW9 | QASQEVKNW (B53:01) | S310T | 97.2 | 98.8 | E |
| | B*53:01 | | | | | | |
| | Cw*04:01 | | | | | | |
| CP36 | A*02:02 | KK9 | KIRLRPGGK (A03:01) | — | 0 | 0 | — |
| | A*03:01 | RK9 | RLRPGGKKK (A03:01) | K28S | 94.5 | 97.5 | MTND |
| | B*35:01 | WF9 | WASRELERF (B35:01) | — | 0 | 0 | — |
| | B*53:01 | SL9 | SLYNTVATL (A02:02) | T84V | 98.2 | 99 | E |
| | Cw*04:01 | TV9 | TLNAWVKVV (A02:02) | V159I | 81.6 | 83.4 | DR |
| | | EW10 | ETINEEAAEW (B53) | — | 0 | 0 | — |
| | | HA9 | HPVHAGPIA (B35:01) | — | 0 | 0 | — |
| | | QW9 | QASQEVKNW (B53:01) | S310T | 70.9 | 78 | MTND |
| CP38 | A*03:01 | KK9 | KIRLRPGGK (A03:01) | K26R | 10.4 | 13.1 | E |
| | A*24:02 | RK9 | RLRPGGKKK (A03:01) | K26R, K28Q | 75.4 | 78.7 | E |
| | B*44:02 | KW9 | KYKLKHIVW (A24:02) | K28Q, K30R, I34L | 98.3 | 99.4 | E |
| | B*81:01 | TL9 | TPQDLNTML (B81:01) | Q182G/S, T186L | 95.7 | 99.4 | MTND |
| | Cw*05:01 | RL11 | RDYVDRFYKTL (B44:02) | — | 0 | 0 | — |
| | Cw*18:01 | AW11 | AEQASQEVKNW (B44:02, Cw5) | S310T | 12.5 | 12.3 | MTND |
| CP39 | A*03:01 | KK9 | KIRLRPGGK (A03:01) | — | 0 | 0 | — |
| | A*29:02 | RK9 | RLRPGGKKK (A03:01) | K28Q | 94.7 | 98.6 | E |
| | B*07:02 | LY9 | LYNTVATLY (A29:02) | Y79F, V82I, | 99 | 98.6 | E |
| | B*15:16 | | | T84V, Y86F | | | |
| | Cw*07:02 | SV9 | SPRTLNAWV (B07:02) | — | 0 | 0 | — |
| | Cw*14:02 | TL9 | TPQDLNTML (B07:02) | — | 0 | 0 | — |
| | | HA9 | HPVHAGPIA (B7) | — | 0 | 0 | — |
| | | GL9 | GPGHKARVL (B07:02) | — | 0 | 0 | — |
| CP40 | A*02:02 | KK9 | KIRLRPGGK (A03:01) | K18R | 39.8 | 40.4 | MTND |
| | A*03:01 | RK9 | RLRPGGKKK (A03:01) | K28T/R | 89.5 | 91.1 | DR |
| | B*15:16 | RL10 | RPGGKKKYKL (B51:01) | K28T/R, K30R | 91.8 | 92.4 | MTND |
| | B*51:01 | SL9 | SLYNTVATL (A02:02, Cw14) | Y79F, T81A, | 99.4 | 99.3 | E |
| | Cw*14:02 | | | V82I, T84V | | | |
| | | TV9 | TLNAWVKVV (A02:02) | V159I | 3.4 | 4.2 | DR |
| CP42 | A*02:01 | KK9 | KIRLRPGGK (A03:01) | — | 0 | 0 | — |
| | A*03:01 | RK9 | RLRPGGKKK (A03:01) | K28Q | 97.5 | 98.9 | E |
| | B*07:02 | SL9 | SLYNTVATL (A02:01) | Y79F, V82I | 98.3 | 98.7 | E |
| | B*38:01 | SV9 | SPRTLNAWV (B07:02) | — | 0 | 0 | — |
| | Cw*07:02 | TV9 | TLNAWVKVV (A02:01) | — | 0 | 0 | — |
| | Cw*12:03 | TL9 | TPQDLNTML (B07:02) | — | 0 | 0 | — |
| | | HA9 | HPVHAGPIA (B7) | — | 0 | 0 | — |
| | | GL9 | GPGHKARVL (B07:02) | — | 0 | 0 | — |
| | | FK10 | FLGKIWPSHK (A02:01) | — | 0 | 0 | — |
| CP47 | A*02:01 | SL9 | SLYNTVATL (A02:01) | Y79F, T81A, | 92.3 | 96.4 | E |
| | | | | V82I, T84V | | | |
| | A*25:01 | QW11 | QAISPRTLNAW (A25:01) | I147L | 96.5 | 96.5 | DR |
| | B*18;01 | TV9 | TLNAWVKVV (A02:01) | — | 0 | 0 | — |
| | Cw*07:01 | EW10 | ETINEEAAEW (A25:01) | — | 0 | 0 | — |
| | Cw*12:03 | FRK10 | FRDYVDRFYK (B18:01) | — | 0 | 0 | — |
| | | FK10 | FLGKIWPSHK (A02:01) | — | 0 | 0 | — |
| CP48 | A*33:03 | LY9 | LYNTYATLY (B44:03) | T81A, V82I, T84V | 94.2 | 98.7 | MTND |
| | A*37:01 | RL11 | RDYVDRFYKTL (B44) | — | 0 | 0 | — |
| | B*44:03 | AW11 | AEQASQEVKNW (B44) | S310T | 24.5 | 27.8 | MTND |
| | Cw*06:02 | | | | | | |
| | Cw*07:06 | | | | | | |
| CP49 | A*01:01 | GK9 | GGKKKYKLK (B08:01) | — | 0 | 0 | — |
| | A*02:01 | EV9 | ELRSLYNTV (B08:01) | R76K, Y79F | 99.2 | 98.5 | MTND |
| | B*08:01 | SL9 | SLYNTVATL (A02:01) | Y79F | 99.2 | 98.5 | E |
| | B*13:02 | VQV9 | VQNLQGQMV (B13) | L138M | 97 | 97.3 | MTND |
| | Cw*06:02 | TV9 | TLNAWVKVV (A02:01) | — | 0 | 0 | — |
| | Cw*07:01 | GI11 | GQMKFPRGSD1 (B13) | — | 0 | 0 | — |
| | | EI8 | EIYKRWII (B08:01) | — | 0 | 0 | — |
| | | DL9 | DCKTILKAL (B08:01) | — | 0 | 0 | — |
| | | R19 | RQANFLGKI (B13) | — | 0 | 0 | — |
| | | FK10 | FLGKIWPSHK (A02:01) | — | 0 | 0 | — |
| CP50 | A*03:01 | KK9 | KIRLRPGGK (A03:01) | — | 0 | 0 | — |
| | A*30:04 | RK9 | RLRPGGKKK (A03:01) | K28Q | 91.8 | 94 | E |
| | B*35:01 | WF9 | WASRELERF (B35:01) | — | 0 | 0 | — |
| | B*49:01 | RY11 | RSLYNTVATLY (A30) | R76K | 97.4 | 98.5 | IE |
| | Cw*04:01 | HA9 | HPVHAGPIA (B35:01) | — | 0 | 0 | — |
| | Cw*07:01 | PY9 | PPIPVGEIY (B35:01) | — | 0 | 0 | — |
| AP01 | A*02:01 | KK9 | KIRLRPGGK (A03:01) | — | 0 | 0 | — |
| | A*03:01 | RK9 | RLRPGGKKK (A03:01) | K28Q | 3.4 | 4.3 | E |
| | B*35:01 | WF9 | WASRELFRF (B35:01) | — | 0 | 0 | — |
| | B*44:02 | SL9 | SLYNTVATL (A02:01) | T84V | 94.4 | 98.7 | E |
| | Cw*04:01 | TV9 | TLNAWVKVV (A02:01) | — | 0 | 0 | — |
| | Cw*05:01 | HA9 | HPVHAGPIA (B35:01) | I223P | 95.2 | 94.7 | MTND |
| | | PY9 | PP1PVGEIY (B35:01) | — | 0 | 0 | — |
| | | RL11 | RDYVDRFYKTL (B44:02) | — | 0 | 0 | — |
| | | AW11 | AEQASQEVKNW (B44:02, Cw5) | N315G | 96.5 | 94.9 | MTND |
| | | FK10 | FLGKIWPSHK (A02:01) | 1 | 0 | 0 | — |
| AP03 | A*24:02 | IK9 | IRLRPGGKK (B27:05) | K26N | 66.1 | 61.5 | MTND |
| | A*24:07 | KW9 | KYKLKHIVW (A24:02) | K28Q | 29.5 | 36.4 | E |
| | B*15:02 | VL8 | VIPMFSAL (C01:02) | — | 0 | 0 | — |
| | B*27:05 | KK10 | KRWIILGLNK (B27:05) | R264K, L268M | 25 | 25.8 | E |
| | Cw*01:02 | YL9 | YVDRFYKTL (C03:03) | — | 0 | 0 | — |
| | Cw*03:03 | | | | | | |
| AP04 | A*02:01 | KW9 | KYKLKHIVW (A24::02) | K30Q/R | 50.4 | 52.9 | MTND |
| | A*24:02 | WF9 | WASRELFRF (B35:01) | F44Y | 7.7 | 7 | MTND |
| | B*14:01 | SL9 | SLYNTVATL (A02:01) | V82I | 11.7 | 12.3 | E |
| | B*35:01 | TV9 | TLNAWVKVV (A02:01) | V1591 | 7.6 | 6.9 | DR |
| | Cw*04:01 | HA9 | HPVHAGPIA (B35:01) | — | 0 | 0 | — |
| | Cw*15:05 | PY9 | PPIPVGEIY (B35:01) | — | 0 | 0 | — |
| | | CC9 | CRAPRKKGC (B14) | K411R | 70.5 | 79.2 | MTND |
| | | FK10 | FLGKIWPSHK (A02:01) | 1437L | 7.7 | 6.9 | MTND |
| AP05 | A*02:01 | RL10 | RPGGKKKYKL, (B51:01) | K28Q | 95.8 | 98.7 | MTND |
| | B*07:02 | SL9 | SLYNTVATL (A02:03) | — | 0 | 0 | — |
| | B*51:01 | SV9 | SPRTLNAWV (B07:02) | — | 0 | 0 | — |
| | Cw*01:02 | TV9 | TLNAWVKVV (A02:01) | V159I | 99.2 | 98.5 | DR |
| | Cw*07:02 | VL8 | VIPMFSAL (Cw01:02) | — | 0 | 0 | — |
| | | TL9 | TPQDLNTML (B07:02) | — | 0 | 0 | — |
| | | HA9 | HPVHAGPIA (B7) | H219P, I223V | 99.4 | 98.7 | MTND |
| | | GL9 | GPGHKARVL (B07:02) | R361K | 6.1 | 18.3 | MTND |
| | | FK10 | FLGKIWPSHK (A02:01) | H441N | 97.2 | 98.1 | MTND |
| AP07 | A*02:07 | RY11 | RSLYNTVATLY(B58) | — | 0 | 0 | — |
| | A*33:03 | VL8 | VIPMFSAL(Cw01:02) | S173T | 96.6 | 97.2 | DR |
| | B*46:01 | TW10 | TSTLQEQIGW(B58:01) | — | 0 | 0 | — |
| | B*58:01 | YL9 | YVDRFYKTL(A02:07) | — | 0 | 0 | — |
| | Cw*01:02 | QW9 | QASQEVKNW(B58) | — | 0 | 0 | — |
| | Cw*03:02 | | | | | | |
| AP08 | A*02:01 | KK9 | KIRLRPGGK (A03:01) | — | 0 | 0 | — |
| | A*03:01 | RK9 | RLRPGGKKK (A03:01) | R28Q | 96.1 | 98.7 | E |
| | B*07:02 | SL9 | SLYNTVATL (A02:01) | — | 0 | 0 | — |
| | Cw*07:02 | SV9 | SPRTLMAWV (B07:02) | — | 0 | 0 | — |
| | | TV9 | TLNAWVKVV (A02:01) | — | 0 | 0 | — |
| | | TL9 | TPQDLNTML (B07:02) | Q182S | 93.1 | 96.5 | MTND |
| | | HA9 | HPVIIAGPIA (B7) | — | 0 | 0 | — |
| | | GL9 | GPGHKARVL (B07:02) | — | 0 | 0 | — |
| | | FK10 | FLGKIWPSHK (A02:01) | — | 0 | 0 | — |
| AP09 | A*03:01 | KK9 | KIRLRPGGK (A03:01) | — | 0 | 0 | — |
| | A*11:01 | RK9 | RLRPGGKKK (A03:01) | — | 0 | 0 | — |
| | B*08:01 | GK9 | GGKKKYKLK (B08:01) | — | 0 | 0 | — |
| | B*14:02 | EV9 | ELRSLYNTV (B08:01) | R76K, V82I | 98.9 | 98.9 | IE |
| | Cw*07 | EI8 | EIYKRWII (B08:01) | — | 0 | 0 | — |
| | Cw*08 | DA9 | DRFYKTLRA(B14:02) | — | 0 | 0 | — |
| | | DL9 | DCKTILKAL (B08:01) | — | 0 | 0 | — |
| | | AK11 | ACQGVGGPGHK(A11:01) | G357S | 98 | 99.1 | SF |
| | | CC9 | CRAPRKKGC (B14) | — | 0 | 0 | — |
| AP10 | A*01:01 | GK9 | GGKKKYKLK (B08:01) | — | 0 | 0 | — |
| | A*02:01 | EV9 | ELRSLYNTV (B08:01) | — | 0 | 0 | — |
| | B*08:01 | SL9 | SLYNTVATL (A02:01) | — | 0 | 0 | — |
| | B*15:01 | TV9 | TLNAWVKVV (A02:01) | — | 0 | 0 | — |
| | Cw*03 | EI8 | EIYKRWII (B08:01) | — | 0 | 0 | — |
| | Cw*07 | GLY9 | GLNKIVRMY(B15:01) | — | 0 | 0 | — |
| | | DL9 | DCKTILKAL (B08:01) | — | 0 | 0 | — |
| | | FK10 | FLGKIWPSHK (A02:01) | G435R | 31.7 | 51.6 | MTND |
| AP11 | A*02:01 | RL10 | RPGGKKKYKL (B51:01) | — | 0 | 0 | — |
| | B*35:01 | WF9 | WASRELERF (B35:01) | — | 0 | 0 | — |
| | B*51:01 | SL9 | SLYNTVATL (A02:01) | — | 0 | 0 | — |
| | Cw*02:02 | TV9 | TLNAWVKVV (A02:01) | — | 0 | 0 | — |
| | Cw*04:01 | HA9 | HPVHAGPIA (B7) | — | 0 | 0 | — |
| | | PY9 | PPIPVGEIY (B35:01) | — | 0 | 0 | — |
| | | FK10 | FLGKIWPSHK (A02:01) | G435R | 2.1 | 6.7 | MTND |
| AP12 | A*01:01 | KK9 | KIRLRPGGK (A03:01) | — | 0 | 0 | — |
| | A*03:01 | RK9 | RLRPGGKKK (A03:01) | — | 0 | 0 | — |
| | B*07:02 | SV9 | SPRTLNAWV (B07:02) | R150K | 2.7 | 5.6 | MTND |
| | Cw*07 | TL9 | TPQDLNTML (B07:02) | — | 0 | 0 | — |
| | | HA9 | HPVHAGPIA (B7) | — | 0 | 0 | — |
| | | GL9 | GPGHKARVL (B07:02) | — | 0 | 0 | — |

¹Each patient's relevant optimal Gag epitopes are based: on reported information in the HIV Molecular Immunology Database, Los Alamos National Laboratory (http://www.luv.lanl.gov/content/immunology/index.html) according to the HLA type.
²Sequences from each subject were aligned to the reference HIV-1 clade B consensus Gag sequence. Variants were determined by the differences from the reference sequence.
³Mutation Type abbreviations: MTND: mutation type not determined; E: documented escape; IE: inferred escape; DR: diminished response; SF: susceptible form.

TABLE 3
Consensus Subtype 8 Nef (NIH AIDS Reagent 5189)
DATA SHEET
HIV-1 Consensus Subtype B Nef (15-mer) Peptides - Complete Set
(Cat# 5189, Lot# 10 - NOTE solubility data is from lot#9)

| | | | | | | Solubility Data | | | |
|---|---|---|---|---|---|---|---|---|---|
| | | | Molecular | | Peptide | | | 10% | |
| | | | Wt. | Purity | Content | | | Acetic | |
| CAT # | Peptide Name | SEQUENCE | Amu | [%] | [%] | Water | PBS | Acid | DMSO |
| 5139 | HIV-1 Consensus Subtype B Nef | MGGKWSKRSVVGWPT | 1676.0 | 96.0 | 71.0 | + | + | + | + |
| 5140 | HIV-1 Consensus Subtype B Nef | WSKRSVVGWPTVRER | 1843.1 | 91.5 | 69.0 | + | + | + | + |
| 5141 | HIV-1 Consensus Subtype B Nef | SVVGWPTVRERMRRA | 1800.1 | 95.9 | 69.0 | + | + | + | + |
| 5142 | HIV-1 Consensus Subtype B Nef | WPTVRERMRRAEPAA | 1826.1 | 95.2 | 71.0 | + | + | + | + |
| 5143 | HIV-1 Consensus Subtype B Nef | RERMRRAEPAADGVG | 1670.9 | 97.4 | 70.0 | + | + | + | + |
| 5144 | HIV-1 Consensus Subtype B Nef | RRAEPAADGVGAVSR | 1511.7 | 94.8 | 74.0 | + | + | + | + |
| 5145 | HIV-1 Consensus Subtype B Nef | PAADGVGAVSRDLEK | 1484.6 | 98.4 | 80.0 | + | + | + | + |
| 5146 | HIV-1 Consensus Subtype B Nef | GVGAVSRDLEKHGAI | 1508.7 | 96.6 | 74.0 | + | + | + | + |
| 5147 | HIV-1 Consensus Subtype B Nef | VSRDLEKHGAITSSN | 1613.8 | 90.8 | 71.0 | + | + | + | + |
| 5148 | HIV-1 Consensus Subtype B Nef | LEKHGAITSSNTAAN | 1513.6 | 96.0 | 76.0 | + | + | + | + |
| 5149 | HIV-1 Consensus Subtype B Nef | GAITSSNTAANNADC | 1409.4 | 96.2 | 80.0 | − | − | − | − |
| 5150 | HIV-1 Consensus Subtype B Nef | SSNTAANNADCAWLE | 1566.6 | 95.0 | 84.0 | − | − | − | − |
| 5151 | HIV-1 Consensus Subtype B Nef | AANNADCAWLEAQEE | 1634.7 | 98.1 | 90.0 | + | + | − | + |
| 5152 | HIV-1 Consensus Subtype B Nef | ADCAWLEAQEEEEVG | 1678.8 | 94.4 | 87.0 | − | − | − | + |
| 5153 | HIV-1 Consensus Subtype B Nef | WLEAQEEEEVGFPVR | 1813.0 | 97.7 | 82.0 | + | − | + | + |
| 5154 | HIV-1 Consensus Subtype B Nef | QEEEEVGFPVRPQVP | 1739.9 | 91.7 | 88.0 | + | + | + | + |
| 5155 | HIV-1 Consensus Subtype B Nef | EVGFPVRPQVPLRPM | 1722.1 | 96.6 | 76.0 | + | + | + | + |
| 5156 | HIV-1 Consensus Subtype B Nef | PVRPQVPLRPMTYKA | 1753.1 | 96.9 | 72.0 | + | + | + | + |
| 5157 | HIV-1 Consensus Subtype B Nef | QVPLRPMTYKAAVDL | 1702.1 | 92.4 | 77.0 | + | + | + | + |

NOTE: Peptides that are difficult to solubilize can almost always be dissolved in DMSO. Once
a peptide is in solution, the DMSO can be slowly diluted with aqueous medium. Care must be
taken to ensure that the peptide does not begin to precipitate out of solution.

| | | | | | | Solubility Data | | | |
|---|---|---|---|---|---|---|---|---|---|
| | | | | | Peptide | | | 10% | |
| | | | M W | Purity | Content | | | Acetic | |
| CAT # | Peptide Name | SEQUENCE | g/mol | [%] | [%] | Water | PBS | Acid | DMSO |
| 5158 | HIV-1 Consensus Subtype B Nef | RPMTYKAAVDLSHFL | 1749.1 | 91.0 | 73.0 | + | + | + | + |
| 5159 | HIV-1 Consensus Subtype B Nef | YKAAVDLSHFLKEKG | 1706.0 | 95.9 | 69.0 | + | + | + | + |
| 5160 | HIV-1 Consensus Subtype B Nef | VDLSHFLKEKGGLEG | 1628.8 | 95.1 | 76.0 | + | + | + | + |
| 5161 | HIV-1 Consensus Subtype B Nef | HFLKEKGGLEGLIYS | 1691.0 | 95.5 | 75.0 | + | + | + | + |
| 5162 | HIV-1 Consensus Subtype B Nef | SKGGLEGLIYSQKRQ | 1705.9 | 97.0 | 74.0 | + | + | + | + |
| 5163 | HIV-1 Consensus Subtype B Nef | LEGLIYSQKRQDILD | 1791.0 | 94.8 | 79.0 | + | + | + | + |
| 5164 | HIV-1 Consensus Subtype B Nef | IYSQKRQDILDLWVY | 1940.2 | 96.8 | 74.0 | + | − | + | + |
| 5165 | HIV-1 Consensus Subtype B Nef | KRQDILDLWVYHTQG | 1872.1 | 95.9 | 75.0 | + | − | + | + |
| 5166 | HIV-1 Consensus Subtype B Nef | ILDLWVYHTGGYFPD | 1867.1 | 96.0 | 81.0 | − | − | + | + |
| 5167 | HIV-1 Consensus Subtype B Nef | WVYHTQGYFPDWQNY | 2004.0 | 95.1 | 75.0 | − | − | + | + |
| 5168 | HIV-1 Consensus Subtype B Nef | TQGYFPDWQNYTPGP | 1770.9 | 95.5 | 78.0 | + | + | + | + |
| 5169 | HIV-1 Consensus Subtype B Nef | FPDWQNYTPGPGIRY | 1811.0 | 95.3 | 74.0 | + | + | + | + |
| 5170 | HIV-1 Consensus Subtype B Nef | QNYTPGPGIRYPLTF | 1723.9 | 97.2 | 76.0 | + | + | + | + |
| 5171 | HIV-1 Consensus Subtype B Nef | PGPGIRYPLTFGWCF | 1711.0 | 96.9 | 79.0 | − | − | + | + |
| 5172 | HIV-1 Consensus Subtype B Nef | IRYPLTFGWCFKLVP | 1840.3 | 96.6 | 80.0 | + | + | + | + |
| 5173 | HIV-1 Consensus Subtype B Nef | LTFGWCFKLVPVEPE | 1765.1 | 95.9 | 82.0 | − | − | + | + |
| 5174 | HIV-1 Consensus Subtype B Nef | WCFKLVPVEPEKVEE | 1832.2 | 97.6 | 81.0 | − | − | + | + |
| 5175 | HIV-1 Consensus Subtype B Nef | LVPVEPEKVEEANEG | 1638.8 | 96.4 | 84.0 | + | + | + | + |
| 5176 | HIV-1 Consensus Subtype B Nef | EPEKVEEANEGENNS | 1674.7 | 96.1 | 83.0 | + | + | + | + |
| 5177 | HIV-1 Consensus Subtype B Nef | VEEANEGENNSLLHP | 1651.7 | 97.2 | 82.0 | + | + | + | + |
| 5178 | HIV-1 Consensus Subtype B Nef | NEGENNSLLHPMSLH | 1691.9 | 95.5 | 77.0 | + | + | + | + |
| 5179 | HIV-1 Consensus Subtype B Nef | NNSLLHPMSLHGMDD | 1680.9 | 96.4 | 70.0 | + | + | + | + |
| 5180 | HIV-1 Consensus Subtype B Nef | LHPMSLHGMDDPERE | 1764.0 | 94.9 | 66.0 | + | + | + | + |
| 5181 | HIV-1 Consensus Subtype B Nef | SLHGMDDPEREVLEW | 1813.0 | 96.1 | 74.0 | + | + | + | + |
| 5182 | HIV-1 Consensus Subtype B Nef | MDDPEREVLEWKFDS | 1896.1 | 95.5 | 74.0 | − | − | + | + |
| 5183 | HIV-1 Consensus Subtype B Nef | EREVLEWKFDSRLAF | 1925.2 | 98.5 | 74.0 | − | − | + | + |
| 5184 | HIV-1 Consensus Subtype B Nef | LEWKFDSRLAFHHMA | 1888.2 | 96.3 | 74.0 | + | − | + | + |
| 5185 | HIV-1 Consensus Subtype B Nef | FDSRLAFHHMARELH | 1867.1 | 93.0 | 71.0 | + | + | + | + |
| 5186 | HIV-1 Consensus Subtype B Nef | LAFHHMARELHPEYY | 1914.2 | 96.6 | 72.0 | + | + | + | + |
| 5187 | HIV-1 Consensus Subtype B Nef | HMARELHPEYYKDC | 1792.0 | 97.0 | 67.0 | + | + | + | + |

Determination of solubility: appr, 0.25 mg of the respective peptide was incubated with 1 ml of
solvent.
Solubility was assessed by visual inspection of the resulting solution/suspension.
“+”: complete dissolution
“−”: incomplete dissolution

Brown 20232 delivery list

| | | | | | MW (1) | | MW (2) | | | |
|---|---|---|---|---|---|---|---|---|---|---|
| | | | | | detected | MW (1) | detected | MW (2) | MW (3) detected | MW (3) |
| Index | JPT-4 | Sequence | Peptide Name | Batch# | [g/mol] | label | [g/mol] | label | [g/mol] | label |
| 1 | 20232_001 | H-MGARASVLSGGELDR-OH | 7872_HIV-1 Consensus B Gag_001 | 070213R-06 | — | — | 759.9 | [M + 2H]2+ | 507 | [M + 3H]3+ |
| 2 | 20232_002 | H-ASVLSGGELDRNEKI-OH | 7872_HIV-1 Consensus B Gag_002 | 070213R-07 | — | — | 830.4 | [M + 2H]2+ | 554.1 | [M + 3H]3+ |
| 3 | 20232_003 | H-SGGKLDRWEKIRLRP-OH | 7872_HIV-1 Consensus B Gag_003 | 190213F-02 | — | — | 906.8 | [M + 2H]2+ | 604.5 | [M + 3H]3+ |
| 4 | 20232_004 | H-LURWEKIRLRPGGKK-OH | 7872_HIV-1 Consensus B Gag_004 | 070213R-08 | — | — | 927 | [M + 2H]2+ | 616.2 | [M + 3H]3+ |
| 5 | 20232_005 | H-EKIRLRPGGKKKYKL-OH | 7872_HIV-1 Consensus B Gag_005 | 070213R-09 | — | — | 906 | [M + 2H]2+ | 605.5 | [M + 3H]3+ |
| 6 | 20232_006 | H-LRPGGKKKYKLKHTV-OH | 7872_HIV-1 Consensus B Gag_006 | 190213F-04 | 1765.6 | [M + H]+ | 883.4 | [M + 2H]2+ | 589.4 | [M + 3H]3+ |
| 7 | 20232_007 | H-GKKKYKLKHIVWASR-OH | 7872_HIV-1 Consensus B Gag_007 | 190213F-06 | — | — | 921.9 | [M + 2H]2+ | 814.8 | [M + 3H]3+ |
| 8 | 20232_008 | H-YKLKHIVWASREKER-OH | 7872_HIV-1 Consensus B Gag_008 | 190213F-08 | — | — | 964.9 | [M + 2H]2+ | 643.8 | [M + 3H]3+ |
| 9 | 20232_009 | H-HIVWASRELKRFAVH-OH | 7872_HIV-1 Consensus B Gag_009 | 190213F-10 | — | — | 913.9 | [M + 2H]2+ | 609.0 | [M + 3H]3+ |
| 10 | 20232_010 | H-ASRELERFAVNPGLL-OH | 7872_HIV-1 Consensus B Gag_010 | 190213K-01 | — | — | 836.4 | [M + 2H]2+ | 658.1 | [M + 3H]3+ |
| 11 | 20232_011 | H-LERFAVNPGLLETSE-OH | 7872_HIV-1 Consensus B Gag_011 | 190213K-03 | 1874.9 | [M + H]+ | 838.1 | [M + 2H]2+ | 659 | [M + 3H]3+ |
| 12 | 20232_012 | H-AVNPGLLETSEGCRQ-OH | 7872_HIV-1 Consensus B Gag_012 | 190213K-05 | 1575.2 | [M + H]+ | 787.8 | [M + 2H]2+ | — | — |
| 13 | 20232_013 | H-GLLETSEGCRQILGQ-OH | 7872_HIV-1 Consensus B Gag_013 | 190213K-07 | 1604.4 | [M + H]+ | 802.4 | [M + 2H]2+ | 535.5 | [M + 3H]3+ |
| 14 | 20232_014 | H-TSEGCRQILGQLQPS-OH | 7872_HIV-1 Consensus B Gag_014 | 190213K-09 | 1816.4 | [M + H]+ | 806.9 | [M + 2H]2+ | 539.7 | [M + 3H]3+ |
| 15 | 20232_015 | H-CRQILGQLQPSLQTG-OH | 7872_HIV-1 Consensus B Gag_015 | 190213K-11 | 1642.8 | [M + H]+ | 821.5 | [M + 2H]2+ | 548.2 | [M + 3H]3+ |
| 16 | 20232_016 | H-LGQLQPSLQTGSRHL-OH | 7872_HIV-1 Consensus B Gag_016 | 190213K-13 | 1000.4 | [M + H]+ | 800.4 | [M + 2H]2+ | — | — |
| 17 | 20232_017 | H-QPSLQTGSEHLRSLY-OH | 7872_HIV-1 Consensus B Gag_017 | 190213K-15 | — | — | 854.4 | [M + 2H]2+ | 570.1 | [M + 3H]3+ |
| 18 | 20232_018 | H-QTGSEELRSLYNTVA-OH | 7872_HIV-1 Consensus B Gag_018 | 190213K-17 | — | — | 834.5 | [M + 2H]2+ | 556.8 | [M + 3H]3+ |
| 19 | 20232_019 | H-EKLRSLYMTVATLYC-OH | 7872_HIV-1 Consensus B Gag_019 | 190213K-19 | 1775.4 | [M + H]+ | 886.4 | [M + 2H]2+ | — | — |
| 20 | 20232_020 | H-SLYNTVATLYCVHQR-OH | 7872_HIV-1 Consensus B Gag_020 | 190213K-21 | — | — | 885 | [M + 2H]2+ | 590.1 | [M + 3H]3+ |
| 21 | 20232_021 | H-TVATLYCVHQRIEVK-OH | 7872_HIV-1 Consensus B Gag_021 | 190213K-23 | 1762.3 | [M + H]+ | 680.8 | [M + 2H]2+ | 587.7 | [M + 3H]3+ |
| 22 | 20232_022 | H-LYCVHQRIEVKDTKE-OH | 7872_HIV-1 Consensus B Gag_022 | 190213K-25 | 1552.4 | [M + H]+ | 931.4 | [M + 2H]2+ | 621.4 | [M + 3H]3+ |
| 23 | 20232_023 | H-HQRIEVKDTKEALEK-OH | 7872_HIV-1 Consensus B Gag_023 | 190213K-27 | — | — | 912.4 | [M + 2H]2+ | 606.8 | [M + 3H]3+ |
| 24 | 20232_024 | H-EVKDTKEALEKIERH-OH | 7872_HIV-1 Consensus B Gag_024 | 190213K-29 | — | — | 895.3 | [M + 2H]2+ | 597.4 | [M + 3H]3+ |
| 25 | 20232_025 | H-TKEALEKIEEEQNKS-OH | 7872_HIV-1 Consensus B Gag_025 | 190213K-31 | — | — | 888.8 | [M + 2H]2+ | 592.7 | [M + 3H]3+ |
| 26 | 20232_026 | H-LEKIEEEQNKSKKKA-OH | 7872_HIV-1 Consensus B Gag_026 | 190213K-33 | 1603.3 | [M + H]+ | 901.8 | [M + 2H]2+ | 601.7 | [M + 3H]3+ |
| 27 | 20232_027 | H-KEEQNKSKKKAQQAA-OH | 7872_HIV-1 Consensus B Gag_027 | 190213K-35 | 1717.8 | [M + H]+ | 859.1 | [M + 2H]2+ | 573.2 | [M + 3H]3+ |
| 28 | 20232_028 | H-NKSKKKAQQAAADTG-OH | 7872_HIV-1 Consensus B Gag_028 | 190213K-37 | 1547.1 | [M + H]+ | 773.7 | [M + 2H]2+ | 516.2 | [M + 3H]3+ |
| 29 | 20232_029 | H-KKAQQAAADTGGSSQ-OH | 7872_HIV-1 Consensus B Gag_029 | 190213K-39 | 1505.2 | [M + H]+ | 753.1 | [M + 2H]2+ | — | — |
| 30 | 20232_030 | H-QAAADTGGSSQVSQN-OH | 7872_HIV-1 Consensus B Gag_030 | 190213K-41 | 1477.7 | [M + H]+ | 739.5 | [M + 2H]2+ | — | — |
| 31 | 20232_031 | H-DTGNSSQVSQNYPIV-OH | 7872_HIV-1 Consensus B Gag_031 | 190213K-43 | 1610.1 | [M + H]+ | 805.2 | [M + 2H]2+ | — | — |
| 32 | 20232_032 | H-SSQVSQNTPIVQNLQ-OH | 7872_HIV-1 Consensus B Gag_032 | 190213K-45 | 1707.8 | [M + H]+ | 853.1 | [M + 2H]2+ | 560 | [M + 3H]3+ |
| 33 | 20232_033 | H-SQNYPTVQNLQGQMV-OH | 7872_HIV-1 Consensus B Gag_033 | 190213K-47 | — | — | 859.8 | [M + 2H]2+ | — | — |
| 34 | 20232_034 | H-PIVQNLQGQMVRQAI-OH | 7872_HIV-1 Consensus B Gag_034 | 220213Z-10 | 1675.5 | [M + H]+ | 838.4 | [M + 2H]2+ | 559.5 | [M + 3H]3+ |
| 35 | 20232_035 | H-NLQGQMVEQAISPRT-OH | 7872_HIV-1 Consensus B Gag_035 | 220213Z-12 | 1682.2 | [M + H]+ | 840.8 | [M + 2H]2+ | 561.5 | [M + 3H]3+ |
| 36 | 20232_036 | H-QMVHQAISPRTLNAW-OH | 7872_HIV-1 Consensus B Gag_036 | 220213Z-14 | — | — | 876.8 | [M + 2H]2+ | 564.8 | [M + 3H]3+ |
| 37 | 20232_037 | H-QAISPRTLNAMVKVV-OH | 7872_HIV-1 Consensus B Gag_037 | 220213Z-16 | — | — | 841.5 | [M + 2H]2+ | 561.5 | [M + 3H]3+ |
| 38 | 20232_038 | H-PRTLNAWVKVVEEKA-OH | 7872_HIV-1 Consensus B Gag_038 | 220213Z-18 | — | — | 870.8 | [M + 2H]2+ | 580.7 | [M + 3H]3+ |
| 39 | 20232_039 | H-NAWVKVVEEKAFSPE-OH | 7872_HIV-1 Consensus B Gag_039 | 220213Z-20 | 1732.5 | [M + H]+ | 866.9 | [M + 2H]2+ | 578.4 | [M + 3H]3+ |
| 40 | 20232_040 | H-KVVEEKAFSFEVIPM-OH | 7872_HIV-1 Consensus B Gag_040 | 220213Z-22 | 1703.5 | [M + H]+ | 851.9 | [M + 2H]2+ | 568.4 | [M + 3H]3+ |
| 41 | 20232_041 | H-EKAPSPEVIPNFSAL-OH | 7872_HIV-1 Consensus B Gag_041 | 220213Z-24 | 1666.5 | [M + H]+ | 833.3 | [M + 2H]2+ | 556.1 | [M + 3H]3+ |
| 42 | 20232_042 | H-SPEVIPMFSALSEGA-OH | 7872_HIV-1 Consensus B Gag_042 | 220213Z-26 | 1535.7 | [M + H]+ | 768 | [M + 2H]2+ | — | — |
| 43 | 20232_043 | H-IPMFSALSEGATPQD-OH | 7872_HIV-1 Consensus B Gag_043 | 220213Z-28 | 1565.2 | [M + H]+ | 762.8 | [M + 2H]2+ | — | — |
| 44 | 20232_044 | H-SALSEGATPQDLNTM-OH | 7872_HIV-1 Consensus B Gag_044 | 220213Z-30 | 1535.2 | [M + H]+ | 768.2 | [M + 2H]2+ | — | — |
| 45 | 20232_045 | H-EGATPQDLNTMLNTV-OH | 7872_HIV-1 Consensus B Gag_045 | 220213Z-32 | 1604.3 | [M + H]+ | 802.7 | [M + 2H]2+ | — | — |
| 46 | 20232_046 | H-PQDLNTNLNTVGGHQ-OH | 7872_HIV-1 Consensus B Gag_046 | 220213Z-34 | 1625.4 | [M + H]+ | 812.8 | [M + 2H]2+ | 542.4 | [M + 3H]3+ |
| 47 | 20232_047 | H-NTNLNTVGGHQAAHQ-OH | 7872_HIV-1 Consensus B Gag_047 | 220213Z-36 | 1574.2 | [M + H]+ | 787.2 | [M + 2H]2+ | — | — |
| 48 | 20232_048 | H-NTVGGHQAAMQMLKE-OH | 7872_HIV-1 Consensus B Gag_048 | 220213Z-38 | 1615.4 | [M + H]+ | 807.8 | [M + 2H]2+ | 539 | [M + 3H]3+ |
| 49 | 20232_049 | H-GHQAAMQMLKETINE-OH | 7872_HIV-1 Consensus B Gag_049 | 220213Z-40 | 1701.8 | [M + H]+ | 851 | [M + 2H]2+ | 567.8 | [M + 3H]3+ |
| 50 | 20232_050 | H-AMQMLKETINEEAAE-OH | 7872_HIV-1 Consensus B Gag_050 | 220213Z-42 | 1709.3 | [M + H]+ | 854.8 | [M + 2H]2+ | — | — |
| 51 | 20232_051 | H-LKETINEEAAENDRL-OH | 7872_HIV-1 Consensus B Gag_051 | 220213Z-44 | — | — | 909.3 | [M + 2H]2+ | 606.5 | [M + 3H]3+ |
| 52 | 20232_052 | H-INBEAAEWDRLHPVH-OH | 7872_HIV1 Consensus B Gag_052 | 220213R-01 | — | — | 908.4 | [M + 2H]2+ | 506 | [M + 3H]3+ |
| 53 | 20232_053 | H-AAEWDRLHFVHAGPI-OH | 7872_HIV1 Consensus B Gag_053 | 040313Y-01 | — | — | 834.9 | [M + 2H]2+ | 557.1 | [M + 3H]3+ |
| 54 | 20232_054 | H-DRLHPVHAGPIAPGO-OH | 7872_HIV1 Consensus B Gag_054 | 040313Y-03 | 1565.4 | [M + H]+ | 782.9 | [M + 2H]2+ | 522.5 | [M + 3H]3+ |
| 55 | 20232_055 | H-PVHAGPIAPGQMREP-OH | 7872_HIV1 Consensus B Gag_055 | 040313Y-05 | 1658.8 | [M + H]+ | 779 | [M + 2H]2+ | 519.8 | [M + 3H]3+ |
| 56 | 20232_056 | H-GPIAPGQMREPRGSD-OH | 7872_HIV1 Consensus B Gag_056 | 040313Y-07 | 1568.3 | [M + H]+ | 784.7 | [M + 2H]2+ | 523.5 | [M + 3H]3+ |
| 57 | 20232_057 | H-PGOMREPRGSDIAGT-OH | 7872_HIV1 Consensus B Gag_057 | 040313Y-09 | 1572.2 | [M + H]+ | 766.7 | [M + 2H]2+ | — | [M + 3H]3+ |
| 58 | 20232_058 | H-REPRGSDIAGTTSTL-OH | 7872_HIV1 Consensus B Gag_058 | 040313Y-11 | — | — | 780.9 | [M + 2H]2+ | 521 | [M + 3H]3+ |
| 59 | 20232_059 | H-GSDIAGTTSTLQEQI-OH | 7872_HIV1 Consensus B Gag_059 | 040313Y-13 | 1521.3 | [M + H]+ | 760.8 | [M + 2H]2+ | — | [M + 3H]3+ |
| 60 | 20232_060 | H-AGITSTLOEOIGWWT-OH | 7872_HIV1 Consensus B Gag_060 | 040313Y-15 | 1625.2 | [M + H]+ | 612.7 | [M + 2H]2+ | — | [M + 3H]3+ |
| 61 | 20232_061 | H-STLQEQIGWMTNNPP-OH | 7872_HIV1 Consensus B Gag_061 | 06021306 | 1716.3 | [M + H]+ | 858.8 | [M + 2H]2+ | — | [M + 3H]3+ |
| 62 | 20232_062 | H-EQIGWMTNNPPIPVG-OH | 7872_HIV1 Consensus B Gag_062 | 040313Y-17 | 1653.4 | [M + H]+ | 826.8 | [M + 2H]2+ | — | [M + 3H]3+ |
| 63 | 20232_063 | H-MMTNNPPIPVGEIYK-OH | 7872_HIV1 Consensus B Gag_063 | 040313Y-19 | 1761.2 | [M + H]+ | 880.5 | [M + 2H]2+ | — | [M + 3H]3+ |
| 64 | 20232_064 | H-NPPIPVGEIYKRWIT-OH | 7872_HIV1 Consensus B Gag_064 | 040313Y-21 | — | — | 898.3 | [M + 2H]2+ | 599.1 | [M + 3H]3+ |
| 65 | 20232_065 | H-PVGEIYKRWIILGLN-OH | 7872_HIV1 Consensus B Gag_065 | 040313Y-23 | 1770.3 | [M + H]+ | 886.4 | [M + 2H]2+ | 591.2 | [M + 3H]3+ |
| 66 | 20232_066 | H-IYKRWIILGLNKIVR-OH | 7872_HIV1 Consensus B Gag_066 | 040313Y-25 | — | — | 943.5 | [M + 2H]2+ | 629.2 | [M + 3H]3+ |
| 67 | 20232_067 | H-WIILGLWKIVRMYSP-OH | 7872_HIV1 Consensus B Gag_067 | 040313Y-27 | 1805.2 | [M + H]+ | 902.5 | [M + 2H]2+ | 601.9 | [M + 3H]3+ |
| 68 | 20232_068 | H-GLNKIVRNYSPTSIL-OH | 7872_HIV1 Consensus B Gag_068 | 040313Y-29 | 1692.7 | [M + H]+ | 846.8 | [M + 2H]2+ | — | [M + 3H]3+ |
| 69 | 20232_069 | H-IVRMYSPTSILDIRQ-OH | 7872_HIV1 Consensus B Gag_069 | 040313Y-31 | 1792.5 | [M + H]+ | 896.8 | [M + 2H]2+ | 598.4 | [M + 3H]3+ |
| 70 | 20232_070 | H-ISPTSILOIRQGPKE-OH | 7872_HIV1 Consensus B Gag_070 | 040313Y-33 | — | — | 852.4 | [M + 2H]2+ | 588.8 | [M + 3H]3+ |
| 71 | 20232_071 | H-SILDIRDGPKEPPRD-OH | 7872_HIV1 Consensus B Gag_071 | 040313Y-35 | — | — | 885.9 | [M + 2H]2+ | 591.1 | [M + 3H]3+ |
| 72 | 20232_072 | H-IRQGPKEPPRDYVDR-OH | 7872_HIV1 Consensus B Gag_072 | 040313Y-37 | — | — | 938.9 | [M + 2H]2+ | 626.1 | [M + 3H]3+ |
| 73 | 20232_073 | H-PKEPPRDIVDRFYET-OH | 7872_HIV1 Consensus B Gag_073 | 040313Y-39 | — | — | 980.9 | [M + 2H]2+ | 654.5 | [M + 3H]3+ |
| 74 | 20232_074 | H-PRDYVDRFYKTLRAE-OH | 7872_HIV1 Consensus B Gag_074 | 270213M-17 | — | — | 990.8 | [M + 2H]2+ | 660.7 | [M + 3H]3+ |
| 75 | 20232_075 | H-VDRFYKTLRAEQASQ-OH | 7872_HIV1 Consensus B Gag_075 | 270213M-19 | — | — | 906.9 | [M + 2H]2+ | 604.7 | [M + 3H]3+ |
| 76 | 20232_076 | H-YKTLRASQASQEVEN-OH | 7872_HIV1 Consensus B Gag_076 | 270213M-21 | — | — | 862.9 | [M + 2H]2+ | 589.1 | [M + 3H]3+ |
| 77 | 20232_077 | H-RAEQASQIVQNWMTE-OH | 7872_HIV1 Consensus B Gag_077 | 270213M-23 | 1807.3 | [M + H]+ | 904.2 | [M + 2H]2+ | — | [M + 3H]3+ |
| 78 | 20232_078 | H-ASQEVKNWMTETLLV-OH | 7872_HIV1 Consensus B Gag_078 | 270213M-25 | 1750.3 | [M + H]+ | 875.3 | [M + 2H]2+ | — | [M + 3H]3+ |
| 79 | 20232_079 | H-VONWMTETLLVQNAN-OH | 7872_HIV1 Consensus B Gag_079 | 270213M-27 | 1763.2 | [M + H]+ | 861.4 | [M + 2H]2+ | — | [M + 3H]3+ |
| 80 | 20232_080 | H-MTETLLVQNANPDCK-OH | 7872_HIV1 Consensus B Gag_080 | 270213M-29 | 1678.4 | [M + H]+ | 838.8 | [M + 2H]2+ | 558.7 | [M + 3H]3+ |
| 81 | 20232_081 | H-LLVQNANPDCKTILK-OH | 7872_HIV1 Consensus B Gag_081 | 270213M-31 | 1672.4 | [M + H]+ | 835.9 | [M + 2H]2+ | 557.7 | [M + 3H]3+ |
| 82 | 20232_082 | H-NANPDCKIILKALGP-OH | 7872_HIV1 Consensus B Gag_082 | 270213M-33 | 1555.8 | [M + H]+ | 778 | [M + 2H]2+ | — | [M + 3H]3+ |
| 83 | 20232_083 | H-DCKTLLKALGPAATL-OH | 7872_HIV1 Consensus B Gag_083 | 270213M-35 | 1515.5 | [M + H]+ | 757.9 | [M + 2H]2+ | 505.7 | [M + 3H]3+ |
| 84 | 20232_084 | H-ILKALGPAATLEEMN-OH | 7872_HIV1 Consensus B Gag_084 | 270213M-37 | 1588.5 | [M + H]+ | 794.4 | [M + 2H]2+ | 530 | [M + 3H]3+ |
| 85 | 20232_085 | H-LGPAATLEEMNTACQ-OH | 7872_HIV1 Consensus B Gag_085 | 270213M-39 | 1566.3 | [M + H]+ | 783.8 | [M + 2H]2+ | — | [M + 3H]3+ |
| 86 | 20232_086 | H-ATLEEMMTACQGVGG-OH | 7872_HIV1 Consensus B Gag_086 | 270213M-41 | 1498.2 | [M + H]+ | 749.7 | [M + 2H]2+ | — | [M + 3H]3+ |
| 87 | 20232_087 | H-EMNTACQGVGGPGHK-OH | 7872_HIV1 Consensus B Gag_087 | 270213M-43 | 1504.3 | [M + H]+ | 751.8 | [M + 2H]2+ | 501.7 | [M + 3H]3+ |
| 88 | 20232_088 | H-ACQGVGGPGHKARVL-OH | 7872_HIV1 Consensus B Gag_088 | 270213M-45 | 1450.4 | [M + H]+ | 725.4 | [M + 2H]2+ | 464 | [M + 3H]3+ |
| 89 | 20232_089 | H-VGGPGHKARVLAEAM-OH | 7872_HIV1 Consensus B Gag_089 | 270213M-47 | 1493.4 | [M + H]+ | 746.9 | [M + 2H]2+ | 498.4 | [M + 3H]3+ |
| 90 | 20232_090 | H-GHKARVLAEAMSQVT-OH | 7872_HIV1 Consensus B Gag_090 | 08031307 | 1599.3 | [M + H]+ | 799.7 | [M + 2H]2+ | — | [M + 3H]3+ |
| 91 | 20232_091 | H-RVLAEAHSQVTNSAT-OH | 7872_HIV1 Consensus B Gag_091 | 150313812 | — | — | 789.6 | [M + 2H]2+ | 526.8 | [M + 3H]3+ |
| 92 | 20232_092 | H-EAMSQVTMSATIMMO-OH | 7872_HIV1 Consensus B Gag_092 | 18031387 | 1642 | [M + H]+ | 821.5 | [M + 2H]2+ | — | [M + 3H]3+ |
| 93 | 20232_093 | H-QVTNSATIMMQRGNF-OH | 7872_HIV1 Consensus B Gag_093 | 18031388 | 1697.2 | [M + H]+ | 846.5 | [M + 2H]2+ | — | [M + 3H]3+ |
| 94 | 20232_094 | H-SATIKMQKGNFRNQR-OH | 7872_HIV1 Consensus B Gag_094 | 14031387 | — | — | 906.2 | [M + 2H]2+ | 804.3 | [M + 3H]3+ |
| 95 | 20232_095 | H-MMQRGNFRNQRKTVK-OH | 7872_HIV1 Consensus B Gag_095 | 080313C11 | — | — | 947.6 | [M + 2H]2+ | 632.4 | [M + 3H]3+ |
| 96 | 20232_096 | H-GNFRNQRKTVKCFMC-OH | 7872_HIV1 Consensus B Gag_096 | 070313C4 | 1815.4 | [M + H]+ | 907.5 | [M + 2H]2+ | 605.3 | [M + 3H]3+ |
| 97 | 20232_097 | H-HORKTVKCFNCGKRG-OH | 7872_HIV1 Consensus B Gag_097 | 070313C5 | — | — | 856.5 | [M + 2H]2+ | 571.5 | [M + 3H]3+ |
| 98 | 20232_098 | H-TVKCFNOGKEGHIAK-OH | 7872_HIV1 Consensus B Gag_098 | 070313C6 | — | — | 817.8 | [M + 2H]2+ | 545.8 | [M + 3H]3+ |
| 99 | 20232_099 | H-FNCGKEGHIAKNCRA-OH | 7872_HIV1 Consensus B Gag_099 | 070313C8 | 1648.2 | [M + H]+ | 824.7 | [M + 2H]2+ | 550.3 | [M + 3H]3+ |
| 100 | 20232_100 | H-KEGHIAKNCRAPRKK-OH | 7872_HIV1 Consensus B Gag_100 | 070313C9 | — | — | 860.1 | [M + 2H]2+ | 579.5 | [M + 3H]3+ |
| 101 | 20232_101 | H-IAKNCRAPKKKGCWK-OH | 7872_HIV1 Consensus B Gag_101 | 070313C10 | 1759.8 | [M + H]+ | 880.4 | [M + 2H]2+ | 587.3 | [M + 3H]3+ |
| 102 | 20232_102 | H-CRAPRKKGCWKCGKE-OH | 7872_HIV1 Consensus B Gag_102 | 25021384 | 1750.5 | [M + H]+ | 876.8 | [M + 2H]2+ | 584.4 | [M + 3H]3+ |
| 103 | 20232_103 | H- [symbol] -OH | 7872_HIV1 Consensus B Gag_103 | 250213B5 | — | — | 8 [symbol] .4 | [M + 2H]2+ | 582.7 | [M + 3H]3+ |
| 104 | 20232_104 | H- [symbol] -OH | 7872_HIV1 Consensus B Gag_104 | 250213B6 | — | — | 877.3 | [M + 2H]2+ | 585.4 | [M + 3H]3+ |
| 105 | 20232_105 | H- [symbol] -OH | 7872_HIV1 Consensus B Gag_105 | 250213B7 | — | — | 859.3 | [M + 2H]2+ | 573.4 | [M + 3H]3+ |
| 106 | 20232_106 | H- [symbol] -OH | 7872_HIV1 Consensus B Gag_106 | 250213B8 | — | — | 859.3 | [M + 2H]2+ | 593.4 | [M + 3H]3+ |
| 107 | 20232_107 | H- [symbol] -OH | 7872_HIV1 Consensus B Gag_107 | 250213B9 | [symbol] .2 | [M + H]+ | 893 | [M + 2H]2+ | 593.7 | [M + 3H]3+ |
| 108 | 20232_108 | H- [symbol] -OH | 7872_HIV1 Consensus B Gag_108 | 250213B10 | — | — | 869.6 | [M + 2H]2+ | [symbol] | [M + 3H]3+ |
| 109 | 20232_109 | H- [symbol] -OH | 7872_HIV1 Consensus B Gag_109 | 260213C2 | 16 [symbol] .2 | [M + H]+ | 847.8 | [M + 2H]2+ | 585.7 | [M + 3H]3+ |
| 110 | 20232_110 | H- [symbol] -OH | 7872_HIV1 Consensus B Gag_110 | 260213C3 | 1728.2 | [M + H]+ | 852.8 | [M + 2H]2+ | 575.7 | [M + 3H]3+ |
| 111 | 20232_111 | H- [symbol] -OH | 7872_HIV1 Consensus B Gag_111 | 260213C4 | — | — | 860.4 | [M + 2H]2+ | 574.1 | [M + 3H]3+ |
| 112 | 20232_112 | H- [symbol] -OH | 7872_HIV1 Consensus B Gag_112 | 260213C5 | 1619.2 | [M + H]+ | 8 [symbol] .0 | [M + 2H]2+ | — | [M + 3H]3+ |
| 113 | 20232_113 | H- [symbol] -OH | 7872_HIV1 Consensus B Gag_113 | 260213C6 | 1658.2 | [M + H]+ | 843.2 | [M + 2H]2+ | — | [M + 3H]3+ |
| 114 | 20232_114 | H- [symbol] -OH | 7872_HIV1 Consensus B Gag_114 | 260213C7 | [symbol] .3 | [M + H]+ | 848. [symbol] | [M + 2H]2+ | — | [M + 3H]3+ |
| 115 | 20232_115 | H- [symbol] -OH | 7872_HIV1 Consensus B Gag_115 | 040313C3 | [symbol] .1 | [M + H]+ | [symbol] 8.7 | [M + 2H]2+ | — | [M + 3H]3+ |
| 116 | 20232_116 | H- [symbol] -OH | 7872_HIV1 Consensus B Gag_116 | 040313C4 | — | — | 872.4 | [M + 2H]2+ | 562.1 | [M + 3H]3+ |
| 117 | 20232_117 | H- [symbol] -OH | 7872_HIV1 Consensus B Gag_117 | 040313C5 | — | — | 848.3 | [M + 2H]2+ | 564.7 | [M + 3H]3+ |
| 118 | 20232_118 | H- [symbol] -OH | 7872_HIV1 Consensus B Gag_118 | [symbol] | — | — | 857.9 | [M + 2H]2+ | 577.4 | [M + 3H]3+ |
| 119 | 20232_119 | H- [symbol] -OH | 7872_HIV1 Consensus B Gag_119 | 280213C8 | 1761.3 | [M + H]+ | 860.4 | [M + 2H]2+ | 587.5 | [M + 3H]3+ |
| 120 | 20232_120 | H- [symbol] -OH | 7872_HIV1 Consensus B Gag_120 | 280213C9 | — | — | 865.9 | [M + 2H]2+ | 577.7 | [M + 3H]3+ |
| 121 | 20232_121 | H- [symbol] -OH | 7872_HIV1 Consensus B Gag_121 | 280213C10 | — | — | 854.4 | [M + 2H]2+ | 576.2 | [M + 3H]3+ |
| 122 | 20232_122 | H- [symbol] -OH | 7872_HIV1 Consensus B Gag_122 | 11031384 | 1861.3 | [M + H]+ | 781.2 | [M + 2H]2+ | — | [M + 3H]3+ |
| 123 | 20232_123 | H- [symbol] -OH | 7872_HIV1 Consensus B Gag_123 | 280213C11 | 1821.1 | [M + H]+ | [symbol] 81.1 | [M + 2H]2+ | — | [M + 3H]3+ |

| | Theor. MW | | | | | | |
|---|---|---|---|---|---|---|---|
| | (average) | Theor. MW + TFA | Exp. MW vs. | | | | |
| Index | [g/mol] | [g/mol] | Theor. MW | Purity [%] | Amount [mg] | Purified | Comment |
| 1 | 1518.72 | 1860.72 | pass | 96.4 | 50 | Y | |
| 2 | 1659.87 | 2001.87 | pass | 90.1 | 50 | Y | |
| 3 | 1812.08 | 2382.08 | pass | 93.1 | 50 | Y | |
| 4 | 1852.21 | 2850.21 | pass | 90.9 | 50 | Y | |
| 5 | 1814.25 | 2726.25 | pass | 87.7 | 50 | Y | |
| 6 | 1785.21 | 2677.21 | pass | 81.7 | 50 | Y | |
| 7 | 1842.25 | 2754.25 | pass | 91.7 | 50 | Y | |
| 8 | 1928.28 | 2612.26 | pass | 88.1 | 50 | Y | |
| 9 | 1827.09 | 2283.09 | pass | 85.7 | 50 | Y | |
| 10 | 1671.94 | 2013.94 | pass | 96.2 | 50 | Y | |
| 11 | 1674.9 | 1902.8 | pass | 81.5 | 50 | Y | |
| 12 | 1573.78 | 1801.76 | pass | 81 | 50 | Y | |
| 13 | 1603.83 | 1831.83 | pass | 90.7 | 50 | Y | |
| 14 | 1616.83 | 1844.83 | pass | 90.4 | 50 | Y | |
| 15 | 1641.92 | 1669.92 | pass | 88.7 | 50 | Y | |
| 16 | 1599.78 | 1713.78 | pass | 93.2 | 50 | Y | |
| 17 | 1707.89 | 1935.69 | pass | 80.4 | 50 | Y | |
| 18 | 1667.82 | 1895.82 | pass | 86.1 | 50 | Y | |
| 19 | 1775.04 | 2003.84 | pass | 82 | 50 | Y | |
| 20 | 1768.04 | 2110.04 | pass | 82.2 | 50 | Y | 5) |
| 21 | 1760.1 | 2216.1 | pass | 85.2 | 50 | Y | |
| 22 | 1861.16 | 2431.16 | pass | 80.4 | 50 | Y | |
| 23 | 1824.08 | 2508.08 | pass | 91.1 | 50 | Y | |
| 24 | 1789.98 | 2245.98 | pass | 94 | 50 | Y | |
| 25 | 1775.95 | 2231.95 | pass | 94.5 | 50 | Y | |
| 26 | 1802.06 | 2486.06 | pass | 81.9 | 50 | Y | 5) |
| 27 | 1716.87 | 2286.67 | pass | 50.4 | 50 | Y | |
| 28 | 1545.71 | 2115.71 | pass | 87.5 | 50 | Y | 5) |
| 29 | 1504.58 | 1846.58 | pass | 81.9 | 50 | Y | 5) |
| 30 | 1477.47 | 1591.47 | pass | 86.5 | 50 | Y | |
| 31 | 1508.69 | 1722.69 | pass | 89.1 | 50 | Y | |
| 32 | 1704.86 | 1818.86 | pass | 84.3 | 50 | Y | 1) |
| 33 | 1716.94 | 1632.94 | pass | 89 | 50 | Y | |
| 34 | 1675.96 | 1903.96 | pass | 82.1 | 50 | Y | |
| 35 | 1679.92 | 2021.92 | pass | 93.5 | 50 | Y | |
| 36 | 1752.03 | 2094.03 | pass | 85.4 | 50 | Y | |
| 37 | 1682 | 2024 | pass | 84.6 | 50 | Y | |
| 38 | 1740.04 | 2196.04 | pass | 95.2 | 50 | Y | |
| 39 | 1732.96 | 2074.96 | pass | 93.9 | 50 | Y | |
| 40 | 1703.04 | 2045.04 | pass | 86.8 | 50 | Y | |
| 41 | 1665.99 | 1893.99 | pass | 58.6 | 50 | Y | |
| 42 | 1534.77 | 1546.77 | pass | 83.8 | 50 | Y | 5) |
| 43 | 1563.77 | 1677.77 | pass | 81.2 | 50 | Y | |
| 44 | 1534.68 | 1646.68 | pass | 81.7 | 50 | Y | |
| 45 | 1603.78 | 1717.78 | pass | 81.6 | 50 | Y | |
| 46 | 1624.79 | 1652.79 | pass | 81.6 | 50 | Y | |
| 47 | 1572.77 | 1800.77 | pass | 84.4 | 50 | Y | |
| 48 | 1614.85 | 1956.85 | pass | 83.4 | 50 | Y | |
| 49 | 1700.95 | 2042.95 | pass | 95.1 | 50 | Y | |
| 50 | 1707.95 | 1935.95 | pass | 88.8 | 50 | Y | |
| 51 | 1817.01 | 2158.01 | pass | 88.5 | 50 | Y | |
| 52 | 1815.98 | 2271.98 | pass | 96 | 50 | Y | |
| 53 | 1668.80 | 2124.89 | pass | 93.85 | 50 | Y | |
| 54 | 1564.78 | 2020.78 | pass | 94.9 | 50 | Y | |
| 55 | 1656.62 | 1896.82 | pass | 84.1 | 50 | Y | |
| 56 | 1567.76 | 1909.76 | pass | 84.5 | 50 | Y | |
| 57 | 1571.75 | 1913.75 | pass | 84.7 | 50 | Y | |
| 58 | 1560.72 | 1902.72 | pass | 82.3 | 50 | Y | 5) |
| 59 | 1520.64 | 1634.64 | pass | 81.7 | 50 | Y | |
| 60 | 1623.82 | 1737.82 | pass | 81.5 | 50 | Y | |
| 61 | 1715.91 | 1829.91 | pass | 85.2 | 50 | Y | |
| 62 | 1852.89 | 1766.89 | pass | 89.5 | 50 | Y | |
| 63 | 1759.06 | 1987.06 | pass | 91 | 50 | Y | |
| 64 | 1795.17 | 2137.17 | pass | 83.4 | 50 | Y | |
| 65 | 1771.14 | 2113.14 | pass | 92.5 | 50 | Y | |
| 66 | 1885.37 | 2455.37 | pass | 90.7 | 50 | Y | |
| 67 | 1803.24 | 2145.24 | pass | 93.4 | 50 | Y | |
| 68 | 1692.06 | 2034.06 | pass | 83.9 | 50 | Y | |
| 69 | 1792.15 | 2134.15 | pass | 83.1 | 50 | Y | |
| 70 | 1703.94 | 2045.94 | pass | 92.3 | 50 | Y | |
| 71 | 1771.03 | 2227.03 | pass | 92.7 | 50 | Y | |
| 72 | 1876.13 | 2446.13 | pass | 87.7 | 50 | Y | |
| 73 | 1961.24 | 2531.24 | pass | 85.5 | 50 | Y | |
| 74 | 1979.26 | 2549.26 | pass | 88 | 50 | Y | |
| 75 | 1812.04 | 2268.04 | pass | 89.5 | 50 | Y | |
| 76 | 1764.97 | 2220.07 | pass | 85.4 | 50 | Y | |
| 77 | 1800.68 | 2148.96 | pass | 85.3 | 50 | Y | |
| 78 | 1749.02 | 1977.02 | pass | 88.6 | 50 | Y | |
| 79 | 1781.02 | 1989.02 | pass | 82.9 | 50 | Y | |
| 80 | 1676.93 | 1904.93 | pass | 89.8 | 50 | Y | |
| 81 | 1670 | 2012 | pass | 88.1 | 50 | Y | 5) |
| 82 | 1654.83 | 1896.83 | pass | 89.7 | 50 | Y | |
| 83 | 1514.86 | 1856.86 | pass | 81 | 50 | Y | |
| 84 | 157.97 | 1815.97 | pass | 92.8 | 50 | Y | |
| 85 | 1565.86 | 1679.86 | pass | 81.8 | 50 | Y | |
| 86 | 1497.73 | 1611.73 | pass | 89.2 | 50 | Y | |
| 87 | 1502.74 | 1844.74 | pass | 84.3 | 50 | Y | |
| 88 | 1449.69 | 1905.69 | pass | 86.7 | 50 | Y | |
| 89 | 1492.76 | 1946.76 | pass | 97.1 | 50 | Y | |
| 90 | 1597.88 | 2053.86 | pass | 81.6 | 50 | Y | |
| 91 | 1577.79 | 1805.79 | pass | 92.9 | 50 | Y | |
| 92 | 1641.9 | 1755.9 | pass | 87.7 | 50 | Y | |
| 93 | 1697.95 | 1925.95 | pass | 86 | 50 | Y | |
| 94 | 1810.09 | 2266.09 | pass | 86.8 | 50 | Y | |
| 95 | 1894.24 | 2578.24 | pass | 84.3 | 50 | Y | 5) |
| 96 | 1815.1 | 2385.1 | pass | 95.3 | 50 | Y | |
| 97 | 1711.97 | 2281.97 | pass | 85.5 | 50 | Y | |
| 98 | 1834.93 | 2204.93 | pass | 86.45 | 50 | Y | |
| 99 | 1847.80 | 2217.89 | pass | 86.6 | 50 | Y | |
| 100 | 1738.07 | 2648.07 | pass | 87.3 | 50 | Y | |
| 101 | 1759.18 | 2557.16 | pass | 82 | 50 | Y | |
| 102 | 1750.13 | 2548.13 | pass | 82.9 | 50 | Y | |
| 103 | 1776.11 | 2578.11 | pass | 86.3 | 50 | Y | |
| 104 | 1754. [symbol] | 2324.04 | pass | 81.3 | 50 | Y | |
| 105 | 1717.9 | 2287.9 | pass | 82.6 | 50 | Y | |
| 106 | 1778 | 2234 | pass | 87.2 | 50 | Y | |
| 107 | 1778.03 | 2120.03 | pass | 91.5 | 50 | Y | |
| 108 | 1739.01 | 2309.01 | pass | 94 | 50 | Y | |
| 109 | 1893. [symbol] | 2253.97 | pass | 89 | 50 | Y | |
| 110 | 17 [symbol] | 2179.28 | pass | 81.7 | 50 | Y | 1) |
| 111 | [symbol] | 2289.84 | pass | 84.7 | 50 | Y | |
| 112 | 1687.83 | 1639.52 | pass | 81.9 | 50 | Y | |
| 113 | [symbol] | 1812.67 | pass | 82.4 | 50 | Y | |
| 114 | [symbol] | 1817.65 | pass | 82.3 | 50 | Y | |
| 115 | 1697.83 | 1825.83 | pass | 81.1 | 50 | Y | |
| 116 | 1743.89 | [symbol] | pass | 86.1 | 50 | Y | |
| 117 | [symbol] | [symbol] | pass | 89.2 | 50 | Y | |
| 118 | 1714.82 | [symbol] | pass | 94.8 | 50 | Y | |
| 119 | 1759.81 | 2101.61 | pass | 94.5 | 50 | Y | |
| 120 | [symbol] | 2828.61 | pass | 93.7 | 50 | Y | |
| 121 | [symbol] | [symbol] | pass | 96.8 | 50 | Y | |
| 122 | 1580.75 | 1788.75 | pass | 92 | 50 | Y | |
| 123 | 1320.44 | 1548.44 | pass | 82.7 | 50 | Y | |

Comments:
1) C [symbol] isomerization of protein
2) no HPLC possible-MALDI performed
3) dissolved in CMSO
4) dissolved in TFA/water
5) dissolved in ACN- [symbol] [symbol]
indicates data missing or illegible when filed

TABLE 5
HIV-1 Consensus B Rev (NIH AIDS Reagent 6445)
DATA SHEET
HIV-1 Consensus B REV (15-mer) Peptides - Complete Set (Cat# 6445, Lot# 7)

| | | | | | Peptide | |
|---|---|---|---|---|---|---|
| Cat # | Peptide Name | Sublot # | Lot # | HPLC Purity | Content | Solubility Data |
| 5991 | HIV-1 Consensus B REV (15-mer) | 5 | 5991 | 91.4% | 77.6% | 1 mg/ml in water |
| | Peptide MAGRSGDSDEELLKT | | | | | |
| 5992 | HIV-1 Consensus B REV (15-mer) | 5 | 5992 | 89.1% | 75.5% | 1 mg/ml in water |
| | Peptide SGDSDEELLKTVRLI | | | | | |
| 5993 | HIV-1 Consensus B REV (15-mer) | 5 | 5993 | 85.2% | 85.5% | 1 mg/ml in water |
| | Peptide DEELLKTVRLIKFLY | | | | | |
| 5994 | HIV-1 Consensus B REV (15-mer) | 5 | 5994 | 92.5% | 80.0% | 1 mg/ml in water |
| | Peptide LKTVRLIKFLYQSNP | | | | | |
| 5995 | HIV-1 Consensus B REV (15-mer) | 5 | 5995 | 89.7% | 80.3% | 1 mg/ml in water |
| | Peptide RLIKPLYQSNPPPSP | | | | | |
| 5996 | HIV-1 Consensus B REV (15-mer) | 5 | 5996 | 95.3% | 89.1% | 1 mg/ml in water |
| | Peptide FLYQSNPPPSPEGTR | | | | | |
| 5997 | HIV-1 Consensus B REV (15-mer) | 5 | 5997 | 93.2% | 65.5% | 1 mg/ml in water |
| | Peptide SNPPPSPEGTRQARR | | | | | |
| 5998 | HIV-1 Consensus B REV (15-mer) | 5 | 5998 | 80.3% | 67.3% | 1 mg/ml in water |
| | Peptide PSPEGTRQARRNRRR | | | | | |
| 5999 | HIV-1 Consensus B REV (15-mer) | 5 | 5999 | 94.5% | 73.0% | 1 mg/ml in water |
| | Peptide GTRQARRNRRRRWRE | | | | | |
| 6000 | HIV-1 Consensus B REV (15-mer) | 5 | 6000 | 89.7% | 67.8% | 1 mg/ml in water |
| | Peptide ARRNRRRRWRERGRQ | | | | | |
| 6001 | HIV-1 Consensus B REV (15-mer) | 5 | 6001 | 91.7% | 67.0% | 1 mg/ml in water |
| | Peptide RRRRWRERQRQIRSI | | | | | |
| 6002 | HIV-1 Consensus B REV (15-mer) | 5 | 6002 | 89.3% | 77.0% | 1 mg/ml in water |
| | Peptide WRERQRQIRSISGWI | | | | | |
| 6003 | HIV-1 Consensus B REV (15-mer) | 5 | 6003 | 82.8% | 74.9% | 1 mg/ml in water |
| | Peptide QRQIRSISGWILSTY | | | | | |
| 6004 | HIV-1 Consensus B REV (15-mer) | 5 | 6004 | 93.0% | 88.7% | 1 mg/ml in water |
| | Peptide RSISGWILSTYLGRP | | | | | |
| 6005 | HIV-1 Consensus B REV (15-mer) | 5 | 6005 | 91.9% | 86.1% | 1 mg/ml in water |
| | Peptide GWILSTYLGRPAEPV | | | | | |
| 6006 | HIV-1 Consensus B REV (15-mer) | 5 | 6006 | 85.0% | 87.5% | 1 mg/ml in water |
| | Peptide STYLGRPAEPVPLQL | | | | | |
| 6007 | HIV-1 Consensus B REV (15-mer) | 5 | 6007 | 83.8% | 82.9% | 1 mg/ml in water |
| | Peptide GRPAEPVPLQLPPLE | | | | | |
| 6008 | HIV-1 Consensus B REV (15-mer) | 5 | 6008 | 84.2% | 89.1% | 1 mg/ml in water |
| | Peptide EPVPLQLPPLERLTL | | | | | |
| 6009 | HIV-1 Consensus B REV (15-mer) | 5 | 6009 | 85.4% | 85.2% | 1 mg/ml in water |
| | Peptide LQLPPLERLTLDCNE | | | | | |
| 6010 | HIV-1 Consensus B REV (15-mer) | 5 | 6010 | 74.8% | 82.8% | 1 mg/ml in water |
| | Peptide PLERLTLDCNEDCGT | | | | | |
| 6011 | HIV-1 Consensus B REV (15-mer) | 5 | 6011 | 83.4% | 82.7% | 1 mg/ml in water |
| | Peptide LTLDCNEDCGTSGTQ | | | | | |
| 6012 | HIV-1 Consensus B REV (15-mer) | 5 | 6012 | 82.1% | 87.4% | 1 mg/ml in water |
| | Peptide CNEDCGTSGTQGVGS | | | | | |
| 6013 | HIV-1 Consensus B REV (15-mer) | 5 | 6013 | 89.9% | 88.0% | 1 mg/ml in water |
| | Peptide CGTSGTQGVGSPQIL | | | | | |
| 6014 | HIV-1 Consensus B REV (15-mer) | 5 | 6014 | 90.5% | 91.0% | 1 mg/ml in water |
| | Peptide GTQGVGSPQILVESP | | | | | |
| 6015 | HIV-1 Consensus B REV (15-mer) | 5 | 6015 | 81.8% | 84.5% | 1 mg/ml in water |
| | Peptide VGSPQILVESPAVLE | | | | | |
| 6016 | HIV-1 Consensus B REV (15-mer) | 5 | 6016 | 82.3% | 75.1% | 1 mg/ml in water |
| | Peptide QILVESPAVLESGTK | | | | | |
| 6017 | HIV-1 Consensus B REV (15-mer) | 5 | 6017 | 89.2% | 85.2% | 1 mg/ml in water |
| | Peptide ESPAVLESGTKEE | | | | | |

NOTE:
Peptides that are difficult to solubilize can almost always be dissolved in DMSO. Once a peptide is in solution, the DMSO can be slowly diluted with aqueous medium. Care must be taken to ensure that the pepside does not begin to precipitate out of solution.

TABLE 6
Consensus B Env (NIH AIDS reagent 9480)

| | | |
|---|---|---|
| Data for Cat# 9480 | | |
| Lot 140223 and #12540 Lot 140347 | Solubility | Solvent |

| | Cat # | Peptide | Sequence | mg/ml | Water | PBS | 10% Acetic Acid | DMSO |
|---|---|---|---|---|---|---|---|---|
| 1 | 8763 | HIV-1 Con B Env | MRVKGIRKNYQHLWR | 0.25 | − | − | − | + |
| 2 | 8764 | HIV-1 Con B Env | GIRKNYQHLWRWGTM | 0.25 | − | − | − | + |
| 3 | 8765 | HIV-1 Con B Env | NYQHLWRWGTMLLGM | 0.25 | − | − | − | + |
| 4 | 8766 | HIV-1 Con B Env | LWRWGTMLLGMLMIC | 0.25 | − | − | − | + |
| 5 | 8767 | HIV-1 Con B Env | GTMLLGMLMICSAAE | 0.25 | − | − | − | + |
| 6 | 8768 | HIV-1 Con B Env | LGMLMICSAAEKLWV | 0.25 | − | − | − | + |
| 7 | 8769 | HIV-1 Con B Env | MICSAAEKLWVTVYY | 0.25 | − | − | − | + |
| 8 | 8770 | HIV-1 Con B Env | AAEKLWVTVYYGVPV | 0.25 | − | − | − | + |
| 9 | 8771 | HIV-1 Con B Env | LWVTVYYGVPVWKEA | 0.25 | − | − | − | + |
| 10 | 8772 | HIV-1 Con B Env | VYYGVPVWKEATTTL | 0.25 | − | − | − | + |
| 11 | 8773 | HIV-1 Con B Env | VPVWKEATTTLFCAS | 0.25 | − | − | − | + |
| 12 | 8774 | HIV-1 Con B Env | KEATTTLFCASDAKA | 0.25 | − | − | − | + |
| 13 | 8775 | HIV-1 Con B Env | TTLFCASDAKAYDTE | 0.25 | − | − | − | + |
| 14 | 8776 | HIV-1 Con B Env | CASDAKAYDTEVHNV | 0.25 | − | − | − | + |
| 15 | 8777 | HIV-1 Con B Env | AKAYDTEVHNVWATH | 0.25 | − | − | − | + |
| 16 | 8778 | HIV-1 Con B Env | DTEVHNVWATHACVP | 0.25 | − | − | − | + |
| 17 | 8779 | HIV-1 Con B Env | HNVWATHACVPTDPN | 0.25 | − | − | − | + |
| 18 | 8780 | HIV-1 Con B Env | ATHACVPTDPNPQEV | 0.25 | − | − | − | + |
| 19 | 8781 | HIV-1 Con B Env | CVPTDPNPQEVVLEN | 0.25 | − | − | − | + |
| 20 | 8782 | HIV-1 Con B Env | DPNPQEVVLENVTEN | 0.25 | − | − | − | + |
| 21 | 8783 | HIV-1 Con B Env | QEVVLENVTENFNMW | 0.25 | − | − | − | + |
| 22 | 8784 | HIV-1 Con B Env | LENVTENFNMWKNNM | 0.25 | − | − | − | + |
| 23 | 8785 | HIV-1 Con B Env | TENFNMWKNNMVEQM | 0.25 | − | − | − | + |
| 24 | 8786 | HIV-1 Con B Env | NMWKNNMVEQMHEDI | 0.25 | − | − | − | + |
| 25 | 8787 | HIV-1 Con B Env | NNMVEQMHEDIISLW | 0.25 | − | − | − | + |
| 26 | 8788 | HIV-1 Con B Env | EQMHEDIISLWDQSL | 0.25 | − | − | − | + |
| 27 | 8789 | HIV-1 Con B Env | EDIISLWDQSLKPCV | 0.25 | − | − | − | + |
| 28 | 8790 | HIV-1 Con B Env | SLWDQSLKPCVKLTP | 0.25 | − | − | − | + |
| 29 | 8791 | HIV-1 Con B Env | QSLKPCVKLTPLCVT | 0.25 | − | − | − | + |
| 30 | 8792 | HIV-1 Con B Env | PCVKLTPLCVTLNCT | 0.25 | − | − | − | + |
| 31 | 8793 | HIV-1 Con B Env | LTPLCVTLNCTDLMN | 0.25 | − | − | − | + |
| 32 | 8794 | HIV-1 Con B Env | CVTLNCTDLMNATNT | 0.25 | − | − | − | + |
| 33 | 8795 | HIV-1 Con B Env | NCTDLMNATNTTNSS | 0.25 | − | − | − | + |
| 34 | 8796 | HIV-1 Con B Env | LMNATNTTNSSSGEK | 0.25 | − | − | − | + |
| 35 | 8797 | HIV-1 Con B Env | TNTTNSSSGEKMEKG | 0.25 | − | − | − | + |
| 36 | 8798 | HIV-1 Con B Env | NSSSGEKMEKGEIKN | 0.25 | − | − | − | |
| 37 | 8799 | HIV-1 Con B Env | GEKMEKGEIKNCSFN | 0.25 | − | − | − | + |
| 38 | 8800 | HIV-1 Con B Env | EKGEIKNCSFNITTS | 0.25 | − | − | − | + |
| 39 | 8801 | HIV-1 Con B Env | IKNCSFNITTSIRDK | 0.25 | − | − | − | + |
| 40 | 8802 | HIV-1 Con B Env | SFNITTSIRDKVQKE | 0.25 | − | − | − | + |
| 41 | 8803 | HIV-1 Con B Env | TTSIRDKVQKEYALF | 0.25 | − | − | − | + |
| 42 | 8804 | HIV-1 Con B Env | RDKVQKEYALFYKLD | 0.25 | − | − | − | + |
| 43 | 8805 | HIV-1 Con B Env | QKEYALFYKLDVVPI | 0.25 | − | − | − | + |
| 44 | 8806 | HIV-1 Con B Env | ALFYKLDVVPIDNDN | 0.25 | − | − | − | + |
| 45 | 8807 | HIV-1 Con B Env | KLDVVPIDNDNTSSY | 0.25 | − | + | − | + |
| 46 | 8808 | HIV-1 Con B Env | VPIDNDNTSSYRLIS | 0.25 | − | − | − | + |
| 47 | 8809 | HIV-1 Con B Env | NDNTSSYRLISCNTS | 0.25 | − | − | − | + |
| 48 | 8810 | HIV-1 Con B Env | SSYRLISCNTSVITQ | 0.25 | − | − | − | + |
| 49 | 8811 | HIV-1 Con B Env | LISCNTSVITQACPK | 0.25 | − | − | − | + |
| 50 | 8812 | HIV-1 Con B Env | NTSVITQACPKVSFE | 0.25 | − | − | − | + |
| 51 | 8813 | HIV-1 Con B Env | ITQACPKVSFEPIPI | 0.25 | − | − | − | + |
| 52 | 8814 | HIV-1 Con B Env | CPKVSFEPIPIHYCA | 0.25 | − | − | − | + |
| 53 | 8815 | HIV-1 Con B Env | SFEPIPIHYCAPAGF | 0.25 | − | − | − | + |
| 54 | 8816 | HIV-1 Con B Env | IPIHYCAPAGFAILK | 0.25 | − | − | − | + |
| 55 | 8817 | HIV-1 Con B Env | YCAPAGFAILKCNDK | 0.25 | − | − | − | + |
| 56 | 8818 | HIV-1 Con B Env | AGFAILKCNDKKFNG | 0.25 | − | − | − | + |
| 57 | 8819 | HIV-1 Con B Env | ILKCNDKKFNGTGPC | 0.25 | − | − | − | + |
| 58 | 8820 | HIV-1 Con B Env | NDKKFNGTGPCTNVS | 0.25 | − | − | − | + |
| 59 | 8821 | HIV-1 Con B Env | FNGTGPCTNVSTVQC | 0.25 | − | − | − | + |
| 60 | 8822 | HIV-1 Con B Env | GPCTNVSTVQCTHGI | 0.25 | − | − | − | + |
| 61 | 8823 | HIV-1 Con B Env | NVSTVQCTHGIRPVV | 0.25 | − | − | − | + |
| 62 | 8824 | HIV-1 Con B Env | VQCTHGIRPVVSTQL | 0.25 | − | − | − | + |
| 63 | 8825 | HIV-1 Con B Env | HGIRPVVSTQLLLNG | 0.25 | − | − | − | + |
| 64 | 8826 | HIV-1 Con B Env | PVVSTQLLLNGSLAE | 0.25 | − | − | − | + |
| 65 | 8827 | HIV-1 Con B Env | TQLLLNGSLAEEEVV | 0.25 | − | − | − | + |
| 66 | 8828 | HIV-1 Con B Env | LNGSLAEEEVVIRSE | 0.25 | − | + | − | + |
| 67 | 8829 | HIV-1 Con B Env | LAEEEVVIRSENFTN | 0.25 | − | + | − | + |
| 68 | 8830 | HIV-1 Con B Env | EVVIRSENFTNNAKT | 0.25 | − | − | − | + |
| 69 | 8831 | HIV-1 Con B Env | RSENFTNNAKTIIVQ | 0.25 | − | − | − | + |
| 70 | 8832 | HIV-1 Con B Env | FTNNAKTIIVQLNES | 0.25 | − | − | − | + |
| 71 | 8833 | HIV-1 Con B Env | AKTIIVQLNESVEIN | 0.25 | − | − | − | + |
| 72 | 8834 | HIV-1 Con B Env | IVQLNESVEINCTRP | 0.25 | − | − | − | + |
| 73 | 8835 | HIV-1 Con B Env | NESVEINCTRPNNNT | 0.25 | − | − | − | + |
| 74 | 8836 | HIV-1 Con B Env | EINCTRPNNNTRKSI | 0.25 | − | − | − | + |
| 75 | 8837 | HIV-1 Con B Env | TRPNNNTRKSIHIGP | 0.25 | − | − | − | + |
| 76 | 8838 | HIV-1 Con B Env | NNTRKSIHIGPGRAF | 0.25 | − | − | − | + |
| 77 | 8839 | HIV-1 Con B Env | KSIHIGPGRAFYTTG | 0.25 | − | − | − | + |
| 78 | 8840 | HIV-1 Con B Env | IGPGRAFYTTGEIIG | 0.25 | − | − | − | + |
| 79 | 8841 | HIV-1 Con B Env | RAFYTTGEIIGDIRQ | 0.25 | − | − | − | + |
| 80 | 8842 | HIV-1 Con B Env | TTGEIIGDIRQAHCN | 0.25 | − | − | − | + |
| 81 | 8843 | HIV-1 Con B Env | IIGDIRQAHCNISRA | 0.25 | − | − | − | + |
| 82 | 8844 | HIV-1 Con B Env | IRQAHCNISRAKWNN | 0.25 | − | − | − | + |
| 83 | 8845 | HIV-1 Con B Env | HCNISRAKWNNTLKQ | 0.25 | − | − | − | + |
| 84 | 8846 | HIV-1 Con B Env | SRAKWNNTLKQIVKK | 0.25 | − | − | − | + |
| 85 | 8847 | HIV-1 Con B Env | WNNTLKQIVKKLREQ | 0.25 | − | − | − | + |
| 86 | 8848 | HIV-1 Con B Env | LKQIVKKLREQFGNK | 0.25 | − | − | − | + |
| 87 | 8849 | HIV-1 Con B Env | VKKLREQFGNKTIVF | 0.25 | − | − | − | + |
| 88 | 8850 | HIV-1 Con B Env | REQFGNKTIVFNQSS | 0.25 | − | − | − | + |
| 89 | 8851 | HIV-1 Con B Env | GNKTIVFNQSSGGDP | 0.25 | − | − | − | + |
| 90 | 8852 | HIV-1 Con B Env | IVFNQSSGGDPEIVM | 0.25 | − | − | − | + |
| 91 | 8853 | HIV-1 Con B Env | QSSGGDPEIVMHSFN | 0.25 | − | − | − | + |
| 92 | 8854 | HIV-1 Con B Env | GDPEIVMHSFNCGGE | 0.25 | − | − | − | + |
| 93 | 8855 | HIV-1 Con B Env | IVMHSFNCGGEFFYC | 0.25 | − | − | − | + |
| 94 | 8856 | HIV-1 Con B Env | SFNCGGEFFYCNTTQ | 0.25 | − | − | − | + |
| 95 | 8857 | HIV-1 Con B Env | GGEFFYCNTTQLFNS | 0.25 | − | − | − | + |
| 96 | 8858 | HIV-1 Con B Env | FYCNTTQLFNSTWNV | 0.25 | − | − | − | + |
| 97 | 8859 | HIV-1 Con B Env | TTQLFNSTWNVNGTW | 0.25 | − | − | − | + |
| 98 | 8860 | HIV-1 Con B Env | FNSTWNVNGTWNNNT | 0.25 | − | − | − | + |
| 99 | 8861 | HIV-1 Con B Env | WNVNGTWNNNTEGND | 0.25 | − | − | − | + |
| 100 | 8862 | HIV-1 Con B Env | GTWNNNTEGNDTITL | 0.25 | − | − | − | + |
| 101 | 8863 | HIV-1 Con B Env | NNTEGNDTITLPCRI | 0.25 | − | − | − | + |
| 102 | 8864 | HIV-1 Con B Env | GNDTITLPCRIKQII | 0.25 | − | − | − | + |
| 103 | 8865 | HIV-1 Con B Env | ITLPCRIKQIINMWQ | 0.25 | − | − | − | + |
| 104 | 8866 | HIV-1 Con B Env | CRIKQIINMWQEVGK | 0.25 | − | − | − | + |
| 105 | 8867 | HIV-1 Con B Env | QIINMWQEVGKAMYA | 0.25 | − | − | − | + |
| 106 | 8868 | HIV-1 Con B Env | MWQEVGKAMYAPPIR | 0.25 | − | − | − | + |
| 107 | 8869 | HIV-1 Con B Env | VGKAMYAPPIRGQIR | 0.25 | − | − | − | + |
| 108 | 8870 | HIV-1 Con B Env | MYAPPIRGQIRCSSN | 0.25 | − | − | − | + |
| 109 | 8871 | HIV-1 Con B Env | PIRGQIRCSSNITGL | 0.25 | − | − | − | + |
| 110 | 8872 | HIV-1 Con B Env | QIRCSSNITGLLLTR | 0.25 | − | − | − | + |
| 111 | 8873 | HIV-1 Con B Env | SSNITGLLLTRDGGN | 0.25 | − | − | − | + |
| 112 | 8874 | HIV-1 Con B Env | TGLLLTRDGGNNNTN | 0.25 | − | − | − | + |
| 113 | 8875 | HIV-1 Con B Env | LTRDGGNNNTNETEI | 0.25 | − | − | − | + |
| 114 | 8876 | HIV-1 Con B Env | GGNNNTNETEIFRPG | 0.25 | − | − | − | + |
| 115 | 8877 | HIV-1 Con B Env | NTNETEIFRPGGGDM | 0.25 | − | − | − | + |
| 116 | 8878 | HIV-1 Con B Env | TEIFRPGGGDMRDNW | 0.25 | − | − | − | + |
| 117 | 8879 | HIV-1 Con B Env | RPGGGDMRDNWRSEL | 0.25 | − | − | − | + |
| 118 | 8880 | HIV-1 Con B Env | GDMRDNWRSELYKYK | 0.25 | − | − | − | + |
| 119 | 8881 | HIV-1 Con B Env | DNWRSELYKYKVVKI | 0.25 | − | − | − | + |
| 120 | 8882 | HIV-1 Con B Env | SELYKYKVVKIEPLG | 0.25 | − | − | + | + |
| 121 | 8883 | HIV-1 Con B Env | KYKVVKIEPLGVAPT | 0.25 | − | − | + | + |
| 122 | 8884 | HIV-1 Con B Env | VKIEPLGVAPTKAKR | 0.25 | − | − | − | + |
| 123 | 8885 | HIV-1 Con B Env | PLGVAPTKAKRRVVQ | 0.25 | − | − | + | + |
| 124 | 8886 | HIV-1 Con B Env | APTKAKRRVVQREKR | 0.25 | − | − | + | + |
| 125 | 8887 | HIV-1 Con B Env | AKRRVVQREKRAVGI | 0.25 | − | − | + | + |
| 126 | 8888 | HIV-1 Con B Env | VVQREKRAVGIGAMF | 0.25 | − | − | − | + |
| 127 | 8889 | HIV-1 Con B Env | EKRAVGIGAMFLGFL | 0.25 | − | − | − | + |
| 128 | 8890 | HIV-1 Con B Env | VGIGAMFLGFLGAAG | 0.25 | − | − | − | + |
| 129 | 8891 | HIV-1 Con B Env | AMFLGFLGAAGSTMG | 0.25 | − | − | − | + |
| 130 | 8892 | HIV-1 Con B Env | GFLGAAGSTMGAASM | 0.25 | − | − | − | + |
| 131 | 8893 | HIV-1 Con B Env | AAGSTMGAASMTLTV | 0.25 | − | − | − | + |
| 132 | 8894 | HIV-1 Con B Env | TMGAASMTLTVQARQ | 0.25 | − | − | − | + |
| 133 | 8895 | HIV-1 Con B Env | ASMTLTVQARQLLSG | 0.25 | − | − | − | + |
| 134 | 8896 | HIV-1 Con B Env | LTVQARQLLSGIVQQ | 0.25 | − | − | − | + |
| 135 | 8897 | HIV-1 Con B Env | ARQLLSGIVQQQNNL | 0.25 | − | − | − | + |
| 136 | 8898 | HIV-1 Con B Env | LSGIVQQQNNLLRAI | 0.25 | − | − | − | + |
| 137 | 8899 | HIV-1 Con B Env | VQQQNNLLRAIEAQQ | 0.25 | − | − | − | + |
| 138 | 8900 | HIV-1 Con B Env | NNLLRAIEAQQHLLQ | 0.25 | − | − | − | + |
| 139 | 8901 | HIV-1 Con B Env | RAIEAQQHLLQLTVW | 0.25 | − | − | − | + |
| 140 | 8902 | HIV-1 Con B Env | AQQHLLQLTVWGIKQ | 0.25 | − | − | − | + |
| 141 | 8903 | HIV-1 Con B Env | LLQLTVWGIKQLQAR | 0.25 | − | − | − | + |
| 142 | 8904 | HIV-1 Con B Env | TVWGIKQLQARVLAV | 0.25 | − | − | − | + |
| 143 | 8905 | HIV-1 Con B Env | IKQLQARVLAVERYL | 0.25 | − | − | − | + |
| 144 | 8906 | HIV-1 Con B Env | QARVLAVERYLKDQQ | 0.25 | − | − | − | + |
| 145 | 8907 | HIV-1 Con B Env | LAVERYLKDQQLLGI | 0.25 | − | − | − | + |
| 146 | 8908 | HIV-1 Con B Env | RYLKDQQLLGIWGCS | 0.25 | − | − | − | + |
| 147 | 8909 | HIV-1 Con B Env | DQQLLGIWGCSGKLI | 0.25 | − | − | − | + |
| 148 | 8910 | HIV-1 Con B Env | LGIWGCSGKLICTTT | 0.25 | − | − | − | + |
| 149 | 8911 | HIV-1 Con B Env | GCSGKLICTTTVPWN | 0.25 | − | − | − | + |
| 150 | 8912 | HIV-1 Con B Env | KLICTTTVPWNASWS | 0.25 | − | − | − | + |
| 151 | 8913 | HIV-1 Con B Env | TTTVPWNASWSNKSL | 0.25 | − | − | − | + |
| 152 | 8914 | HIV-1 Con B Env | PWNASWSNKSLDEIW | 0.25 | − | − | − | + |
| 153 | 8915 | HIV-1 Con B Env | SWSNKSLDEIWDNMT | 0.25 | − | − | − | + |
| 154 | 8916 | HIV-1 Con B Env | KSLDEIWDNMTWMEW | 0.25 | − | − | − | + |
| 155 | 8917 | HIV-1 Con B Env | EIWDNMTWMEWEREI | 0.25 | − | − | − | + |
| 156 | 8918 | HIV-1 Con B Env | NMTWMEWEREIDNYT | 0.25 | − | − | − | + |
| 157 | 8919 | HIV-1 Con B Env | MEWEREIDNYTSLIY | 0.25 | − | − | − | + |
| 158 | 8920 | HIV-1 Con B Env | REIDNYTSLIYTLIE | 0.25 | − | − | − | + |
| 159 | 8921 | HIV-1 Con B Env | NYTSLIYTLIEESQN | 0.25 | − | − | − | + |
| 160 | 8922 | HIV-1 Con B Env | LIYTLIEESQNQQEK | 0.25 | − | − | − | + |
| 161 | 8923 | HIV-1 Con B Env | LIEESQNQQEKNEQE | 0.25 | − | − | − | + |
| 162 | 8924 | HIV-1 Con B Env | SQNQQEKNEQELLEL | 0.25 | − | − | − | + |
| 163 | 8925 | HIV-1 Con B Env | QEKNEQELLELDKWA | 0.25 | − | − | − | + |
| 164 | 8926 | HIV-1 Con B Env | EQELLELDKWASLWN | 0.25 | − | − | − | + |
| 165 | 8927 | HIV-1 Con B Env | LELDKWASLWNWFDI | 0.25 | − | − | − | + |
| 166 | 8928 | HIV-1 Con B Env | KWASLWNWFDITNWL | 0.25 | − | − | − | + |
| 167 | 8929 | HIV-1 Con B Env | LWNWFDITNWLWYIK | 0.25 | − | − | − | + |
| 168 | 8930 | HIV-1 Con B Env | FDITNWLWYIKIFIM | 0.25 | − | − | − | + |
| 169 | 8931 | HIV-1 Con B Env | NWLWYIKIFIMIVGG | 0.25 | − | − | − | + |
| 170 | 8932 | HIV-1 Con B Env | YIKIFIMIVGGLIGL | 0.25 | − | − | − | + |
| 171 | 8933 | HIV-1 Con B Env | FIMIVGGLIGLRIVF | 0.25 | − | − | − | + |
| 172 | 8934 | HIV-1 Con B Env | VGGLIGLRIVFAVLS | 0.25 | − | − | − | + |
| 173 | 8935 | HIV-1 Con B Env | IGLRIVFAVLSIVNR | 0.25 | − | − | − | + |
| 174 | 8936 | HIV-1 Con B Env | IVFAVLSIVNRVRQG | 0.25 | − | − | − | + |
| 175 | 8937 | HIV-1 Con B Env | VLSIVNRVRQGYSPL | 0.25 | − | − | − | + |
| 176 | 8938 | HIV-1 Con B Env | VNRVRQGYSPLSFQT | 0.25 | − | − | − | + |
| 177 | 8939 | HIV-1 Con B Env | RQGYSPLSFQTRLPA | 0.25 | − | − | − | + |
| 178 | 8940 | HIV-1 Con B Env | SPLSFQTRLPAPRGP | 0.25 | − | − | − | + |
| 179 | 8941 | HIV-1 Con B Env | FQTRLPAPRGPDRPE | 0.25 | − | − | − | + |
| 180 | 8942 | HIV-1 Con B Env | LPAPRGPDRPEGIEE | 0.25 | − | − | − | + |
| 181 | 8943 | HIV-1 Con B Env | RGPDRPEGIEEEGGE | 0.25 | − | + | − | + |
| 182 | 8944 | HIV-1 Con B Env | RPEGIEEEGGERDRD | 0.25 | − | + | − | + |
| 183 | 8945 | HIV-1 Con B Env | IEEEGGERDRDRSGR | 0.25 | − | + | − | + |
| 184 | 8946 | HIV-1 Con B Env | GGERDRDRSGRLVDG | 0.25 | − | + | − | + |
| 185 | 8947 | HIV-1 Con B Env | DRDRSGRLVDGFLAL | 0.25 | − | + | − | + |
| 186 | 8948 | HIV-1 Con B Env | SGRLVDGFLALIWDD | 0.25 | − | − | − | + |
| 187 | 8949 | HIV-1 Con B Env | VDGFLALIWDDLRSL | 0.25 | − | − | − | + |
| 188 | 8950 | HIV-1 Con B Env | LALIWDDLRSLCLFS | 0.25 | − | − | − | + |
| 189 | 8951 | HIV-1 Con B Env | WDDLRSLCLFSYHRL | 0.25 | − | − | − | + |
| 190 | 8952 | HIV-1 Con B Env | RSLCLFSYHRLRDLL | 0.25 | − | − | − | + |
| 191 | 8953 | HIV-1 Con B Env | LFSYHRLRDLLLIVT | 0.25 | − | − | − | + |
| 192 | 8954 | HIV-1 Con B Env | HRLRDLLLIVTRIVE | 0.25 | − | − | − | + |
| 193 | 8955 | HIV-1 Con B Env | DLLLIVTRIVELLGR | 0.25 | − | − | − | + |
| 194 | 8956 | HIV-1 Con B Env | IVTRIVELLGRRGWE | 0.25 | − | − | − | + |
| 195 | 8957 | HIV-1 Con B Env | IVELLGRRGWEVLKY | 0.25 | − | − | − | + |
| 196 | 8958 | HIV-1 Con B Env | LGRRGWEVLKYWWNL | 0.25 | − | − | − | + |
| 197 | 8959 | HIV-1 Con B Env | GWEVLKYWWNLLQYW | 0.25 | − | − | − | + |
| 198 | 8960 | HIV-1 Con B Env | LKYWWNLLQYWSQEL | 0.25 | − | − | − | + |
| 199 | 8961 | HIV-1 Con B Env | WNLLQYWSQELKNSA | 0.25 | − | − | − | + |
| 200 | 8962 | HIV-1 Con B Env | QYWSQELKNSAVSLL | 0.25 | − | − | − | + |
| 201 | 8963 | HIV-1 Con B Env | QELKNSAVSLLNATA | 0.25 | − | − | − | + |
| 202 | 8964 | HIV-1 Con B Env | NSAVSLLNATAIAVA | 0.25 | − | − | − | + |
| 203 | 8965 | HIV-1 Con B Env | SLLNATAIAVAEGTD | 0.25 | − | − | − | + |
| 204 | 8966 | HIV-1 Con B Env | ATAIAVAEGTDRVIE | 0.25 | − | − | − | + |
| 205 | 8967 | HIV-1 Con B Env | AVAEGTDRVIEVVQR | 0.25 | − | − | − | + |
| 206 | 8968 | HIV-1 Con B Env | GTDRVIEVVQRACRA | 0.25 | − | − | − | + |
| 207 | 8969 | HIV-1 Con B Env | VIEVVQRACRAILH1 | 0.25 | − | − | − | + |
| 208 | 8970 | HIV-1 Con B Env | VQRACRAILHIPRRI | 0.25 | − | − | + | + |
| 209 | 8971 | HIV-1 Con B Env | CRAILHIPRRIRQGL | 0.25 | − | − | + | + |
| 210 | 8972 | HIV-1 Con B Env | LHIPRRIRQGLERAL | 0.25 | + | − | + | + |
| 211 | 8973 | HIV-1 Con B Env | RRIRQGLERALL | 0.25 | − | − | + | + |

Molecular Weight and Purity Data:
Catalog #9480 Lot 140223 and #12540 Lot 140347

| | | | | | MW (1) | | MW (2) |
|---|---|---|---|---|---|---|---|
| | | | | | detected | | detected |
| | JPT-# | Sequence | Peptide Name | Batch# | [g/mol] | MW (1) label | [g/mol] |
| 1 | 23651_294 | H-MRVKGIRKNYQHLWR-OH | 8763_HIV-1 Con B Env_001 | 270114F-04 | — | — | 993.4 |
| 2 | 23651_295 | H-GIRKNYQHLWRWGTM-OH | 8764_HIV-1 Con B Env_002 | 270114F-06 | — | — | 976.6 |
| 3 | 23651_296 | H-NYQHLWRWGTMLLGM-OH | 8765_HIV-1 Con B Env_003 | 270114F-08 | 1906.2 | [M + H]+ | 953.7 |
| 4 | 23651_297 | H-LWRWGTMLLGMLMIC-OH | 8766_HIV-1 Con B Env_004 | 270114F-10 | 1824.7 | [M + H]+ | 912.7 |
| 5 | 23651_298 | H-GTMLLGMLMICSAAE-OH | 8767_HIV-1 Con B Env_005 | 270114F-12 | 1540.01 | [M + H]+ | 1562.55 |
| 6 | 23651_299 | H-LGMLMICSAAEKLWV-OH | 8768_HIV-1 Con B Env_006 | 270114F-14 | 1665.7 | [M + H]+ | 833.0 |
| 7 | 23651_300 | H-MICSAAEKLWVTVYY-OH | 8769_HIV-1 Con B Env_007 | 270114F-16 | 1777.8 | [M + H]+ | 889.0 |
| 8 | 23651_301 | H-AAEKLWVTVYYGVPV-OH | 8770_HIV-1 Con B Env_008 | 270114F-18 | 1695.7 | [M + H]+ | 848.1 |
| 9 | 23651_302 | H-LWVTVYYGVPVWKEA-OH | 8771_HIV-1 Con B Env_009 | 270114F-20 | 1809.8 | [M + H]+ | 905.6 |
| 10 | 23651_303 | H-VYYGVPVWKEATTTL-OH | 8772_HIV-1 Con B Env_010 | 270114F-22 | 1726.8 | [M + H]+ | 64.1 |
| 11 | 23651_304 | H-VPVWKEATTTLFCAS-OH | 8773_HIV-1 Con B Env_011 | 270114F-24 | 1654.2 | [M + H]+ | 827.2 |
| 12 | 23651_305 | H-KEATTTLFCASDAKA-OH | 8774_HIV-1 Con B Env_012 | 270114F-26 | 1558.6 | [M + H]+ | 779.0 |
| 13 | 23651_306 | H-TTLFCASDAKAYDTE-OH | 8775_HIV-1 Con B Env_013 | 270114F-28 | 1636.6 | [M + H]+ | 818.5 |
| 14 | 23651_307 | H-CASDAKAYDTEVHNV-OH | 8776_HIV-1 Con B Env_014 | 270114F-30 | 1624.6 | [M + H]+ | 812.0 |
| 15 | 23651_308 | H-AKAYDTEVHNVWATH-OH | 8777_HIV-1 Con B Env_015 | 270114F-32 | 1743.1 | [M + H]+ | 871.7 |
| 16 | 23651_309 | H-DTEVHNVWATHACVP-OH | 8778_HIV-1 Con B Env_016 | 270114F-34 | 1679.3 | [M + H]+ | 840.2 |
| 17 | 23651_310 | H-HNVWATHACVPTDPN-OH | 8779_HIV-1 Con B Env_017 | 270114F-36 | 1662.7 | [M + H]+ | 831.5 |
| 18 | 23651_311 | H-ATHACVPTDPNPQEV-OH | 8780_HIV-1 Con B Env_018 | 270114F-38 | 1578.6 | [M + H]+ | 790.0 |
| 19 | 23651_312 | H-CVPTDPNPQEVVLEN-OH | 8781_HIV-1 Con B Env_019 | 270114F-40 | 1655.6 | [M + H]+ | 827.5 |
| 20 | 23651_313 | H-DPNPQEVVLENVTEN-OH | 8782_HIV-1 Con B Env_020 | 270114F-42 | 1696.8 | [M + H]+ | 849.0 |
| 21 | 23651_314 | H-QEVVLENVTENFNMW-OH | 8783_HIV-1 Con B Env_021 | 270114F-44 | 1853.7 | [M + H]+ | 927.0 |
| 22 | 23651_315 | H-LENVTENFNMWKNNM-OH | 8784_HIV-1 Con B Env_022 | 270114F-46 | 1885.7 | [M + H]+ | 942.6 |
| 23 | 23651_316 | H-TENFNMWKNNMVEQM-OH | 8785_HIV-1 Con B Env_023 | 270114F-48 | 1915.7 | [M + H]+ | 959.0 |
| 24 | 23651_317 | H-NMWKNNMVEQMHEDI-OH | 8786_HIV-1 Con B Env_024 | 300114C3 | — | — | 960.0 |
| 25 | 23651_318 | H-NNMVEQMHEDIISLW-OH | 8787_HIV-1 Con B Env_025 | 300114C4 | 1860.7 | [M + H]+ | 930.0 |
| 26 | 23651_319 | H-EQMHEDIISLWDQSL-OH | 8788_HIV-1 Con B Env_026 | 300114C5 | 1845.6 | [M + H]+ | 922.5 |
| 27 | 23651_320 | H-EDIISLWDQSLKPCV-OH | 8789_HIV-1 Con B Env_027 | 300114C6 | 1746.7 | [M + H]+ | 873.5 |
| 28 | 23651_321 | H-SLWDQSLKPCVKLTP-OH | 8790_HIV-1 Con B Env_028 | 300114C10 | 1716.8 | [M + H]+ | 858.0 |
| 29 | 23651_322 | H-QSLKPCVKLTPLCVT-OH | 8791_HIV-1 Con B Env_029 | 310114B4 | 1630.7 | [M + H]+ | 815.6 |
| 30 | 23651_323 | H-PCVKLTPLCVTLNCT-OH | 8792_HIV-1 Con B Env_030 | 310114B5 | 1604.7 | [M + H]+ | 803.0 |
| 31 | 23651_324 | H-LTPLCVTLNCTDLMN-OH | 8793_HIV-1 Con B Env_031 | 310114B6 | 1651.7 | [M + H]+ | 826.5 |
| 32 | 23651_325 | H-CVTLNCTDLMNATNT-OH | 8794_HIV-1 Con B Env_032 | 310114B7 | 1615.6 | [M + H]+ | 807.5 |
| 33 | 23651_326 | H-NCTDLMNATNTTNSS-OH | 8795_HIV-1 Con B Env_033 | 310114B11 | 1588.5 | [M + H]+ | 974.0 |
| 34 | 23651_327 | H-LMNATNTTNSSSGEK-OH | 8796_HIV-1 Con B Env_034 | 310114C11 | 1555.6 | [M + H]+ | 778.0 |
| 35 | 23651_328 | H-TNTTNSSSGEKMEKG-OH | 8797_HIV-1 Con B Env_035 | 310114C12 | 1570.6 | [M + H]+ | 786.0 |
| 36 | 23651_329 | H-NSSSGEKMEKGEIKN-OH | 8798_HIV-1 Con B Env_036 | 310114Y-15 | 1639.9 | [M + H]+ | 816.6 |
| 37 | 23651_330 | H-GEKMEKGEIKNCSFN-OH | 8799_HIV-1 Con B Env_037 | 310114Y-17 | 1713.7 | [M + H]+ | 857.5 |
| 38 | 23651_331 | H-EKGEIKNCSFNITTS-OH | 8800_HIV-1 Con B Env_038 | 310114Y-19 | 1670.7 | [M + H]+ | 836.0 |
| 39 | 23651_332 | H-IKNCSFNITTSIRDK-OH | 8801_HIV-1 Con B Env_039 | 310114Y-22 | 1739.8 | [M + H]+ | 870.6 |
| 40 | 22651_333 | H-SFNITTSIRDKVQKE-OH | 8802_HIV-1 Con B Env_040 | 310114Y-23 | 1765.7 | [M + H]+ | 883.6 |
| 41 | 22651_334 | H-TTSIRDKVQKEVALF-OH | 8803_HIV-1 Con B Env_041 | 310114Y-25 | — | — | 900.1 |
| 42 | 22651_335 | H-ROKVQKEYALFYKLD-OH | 8804_HIV-1 Con B Env_042 | 310114Y-27 | 1917.3 | [M + H]+ | 959.2 |
| 43 | 22651_336 | H-QKEYALFYKLDVVPI-OH | 8805_HIV-1 Con B Env_043 | 310114Y-29 | 1827.8 | [M + H]+ | 913.6 |
| 44 | 22651_337 | H-ALFYKLDVVPIDNDN-OH | 8806_HIV-1 Con B Env_044 | 310114Y-31 | 1736.3 | [M + H]+ | 868.7 |
| 45 | 22651_338 | H-KLDVVPIDNDNTSSV-OH | 8807_HIV-1 Con B Env_045 | 310114Y-33 | 1680.7 | [M + H]+ | 840.5 |
| 46 | 22651_339 | H-VPIONDNTSSYRLIS-OH | 8808_HIV-1 Con B Env_046 | 310114Y-35 | 1693.7 | [M + H]+ | 847.5 |
| 47 | 22651_340 | H-NDNTSSYRLISCNTS-OH | 8809_HIV-1 Con B Env_047 | 030214Z-18 | 1675.6 | [M + H]+ | 838.0 |
| 48 | 22651_341 | H-SSYRLISONTSVITQ-OH | 8810_HIV-1 Con B Env_048 | 030214Z-20 | 1673.7 | [M + H]+ | 836.6 |
| 49 | 22651_342 | H-LISCNTSVITQACPK-OH | 8811_HIV-1 Con B Env_049 | 030214Z-22 | 1578.7 | [M + H]+ | 789.6 |
| 50 | 22651_343 | H-NTSVITQACPKVSFE-OH | 8812_HIV-1 Con B Env_050 | 030214Z-24 | 1625.7 | [M + H]+ | 812.5 |
| 51 | 22651_344 | H-ITQACPKVSFEPIPI-OH | 8813_HIV-1 Con B Env_051 | 030214Z-26 | 1643.7 | [M + H]+ | 822.1 |
| 52 | 22651_345 | H-CPKVSFEPIPIHYCA-OH | 8814_HIV-1 Con B Env_052 | 050214V-01 | 1704.7 | [M + H]+ | 853.0 |
| 53 | 22651_346 | H-SFEPIPIHYCAPAGF-OH | 8815_HIV-1 Con B Env_053 | 050214V-02 | 1650.2 | [M + H]+ | 825.2 |
| 54 | 22651_347 | H-IPIHYCAPAGFAILK-OH | 8816_HIV-1 Con B Env_054 | 050214V-03 | 1615.3 | [M + H]+ | 807.7 |
| 55 | 22651_348 | H-YCAPAGFAILKCNDK-OH | 8817_HIV-1 Con B Env_055 | 050214V-04 | 1614.7 | [M + H]+ | 807.5 |
| 56 | 22651_349 | H-AGFAILKCNDKKFNG-OH | 8818_HIV-1 Con B Env_056 | 050214V-05 | 1627.2 | [M + H]+ | 813.7 |
| 57 | 22651_350 | H-ILKCNDKKFNGTGPC-OH | 8819_HIV-1 Con B Env_057 | 050214V-06 | 1638.7 | [M + H]+ | 819.5 |
| 58 | 22651_351 | H-NDKKFNGTGPCTNVS-OH | 8820_HIV-1 Con B Env_058 | 050214V-07 | 1582.5 | [M + H]+ | 791.5 |
| 59 | 22651_352 | H-FNGTGPCTNVSTVQC-OH | 8821_HIV-1 Con B Env_059 | 050214V-08 | 1529.6 | [M + H]+ | 764.5 |
| 60 | 22651_353 | H-GPCTNVSTVQCTHGI-OH | 8822_HIV-1 Con B Env_060 | 050214V-09 | 1517.6 | [M + H]+ | 759.0 |
| 61 | 22651_354 | H-NVSTVQCTHGIRPVV-OH | 8823_HIV-1 Con B Env_061 | 050214V-10 | 1610.7 | [M + H]+ | 805.5 |
| 62 | 22651_355 | H-VQCTHGIRPVVSTQL-OH | 8824_HIV-1 Con B Env_062 | 050214V-11 | 1638.4 | [M + H]+ | 819.7 |
| 63 | 22651_356 | H-HGIRPVVSTQLLLNG-OH | 8825_HIV-1 Con B Env_063 | 050214V-12 | 1605.8 | [M + H]+ | 802.6 |
| 64 | 22651_357 | H-PVVSTQLLLNGSLAE-OH | 8826_HIV-1 Con B Env_064 | 050214V-13 | 1542.7 | [M + H]+ | 771.0 |
| 65 | 22651_358 | H-TQLLLNGSLAEEEVV-OH | 8827_HIV-1 Con B Env_065 | 050214V-14 | 1615.7 | [M + H]+ | 808.5 |
| 66 | 22651_359 | H-LNGSLAEEEVVIRSE-OH | 8828_HIV-1 Con B Env_066 | 050214V-15 | 1645.7 | [M + H]+ | 823.0 |
| 67 | 22651_360 | H-LAEEEVVIRSENFTN-OH | 8829_HIV-1 Con B Env_067 | 050214V-16 | 1751.7 | [M + H]+ | 875.6 |
| 68 | 22651_361 | H-EVVIRSENFTNNAKT-OH | 8830_HIV-1 Con B Env_068 | 050214V-17 | 1721.7 | [M + H]+ | 861.5 |
| 69 | 22651_362 | H-RSENFTNNAKTIIVQ-OH | 8631_HIV-1 Con B Env_069 | 050214V-18 | 1734.8 | [M + H]+ | 868.1 |
| 70 | 22651_363 | H-FTNNAKTIIVCLNES-OH | 8832_HIV-1 Con B Env_070 | 050214V-19 | 1693.7 | [M + H]+ | 846.5 |
| 71 | 22651_364 | H-AKTIIVQLNESVEIN-OH | 8833_HIV-1 Con B Env_071 | 050214V-20 | 1672.60 | [M + H]+ | 1695.08 |
| 72 | 22651_365 | H-IVQLNESVEINCTRP-OH | 8834_HIV-1 Con B Env_072 | 050214V-21 | 1715.7 | [M + H]+ | 858.0 |
| 73 | 22651_366 | H-NESVEINCTRPNNNT-OH | 8835_HIV-1 Con B Env_073 | 050214V-22 | 1705.6 | [M + H]+ | 853.0 |
| 74 | 22651_367 | H-EINCTRPNNNTRKSI-OH | 8836_HIV-1 Con B Env_074 | 050214V-23 | 1759.8 | [M + H]+ | 880.6 |
| 75 | 22651_368 | H-TRPNNNTRKSIHIGP-OH | 8837_HIV-1 Con B Env_075 | 050214V-24 | — | — | 853.1 |
| 76 | 22651_369 | H-NNTRKSIHIGPGRAF-OH | 8838_HIV-1 Con B Env_076 | 050214V-25 | — | — | 834.6 |
| 77 | 22651_370 | H-KSIHIGPGRAFYTTG-OH | 8839_HIV-1 Con B Env_077 | 050214V-26 | 1605.7 | [M + H]+ | 803.0 |
| 78 | 22651_371 | H-IGPGRAFYTTGEIIG-OH | 8840_HIV-1 Con B Env_078 | 050214V-27 | 1551.7 | [M + H]+ | 776.6 |
| 79 | 22651_372 | H-RAFYTTGEIIGDlRQ-OH | 8841_HIV-1 Con B Env_079 | 050214V-28 | 1740.7 | [M + H]+ | 870.6 |
| 80 | 22651_373 | H-TTGEIIGDIRQAHCN-OH | 8842_HIV-1 Con B Env_080 | 050214V-29 | 1629.2 | [M + H]+ | 814.7 |
| 81 | 22651_374 | H-IIGDIRQAHCNISRA-OH | 8843_HIV-1 Con B Env_081 | 050214V-30 | 1669.2 | [M + H]+ | 834.2 |
| 82 | 22651_375 | H-IRQAKCNISRAKWNN-OH | 8844_HIV-1 Con B Env_082 | 050214V-31 | — | — | 906.1 |
| 83 | 22651_376 | H-HCNISRAKWNNTLKQ-OH | 8845_HIV-1 Con B Env_083 | 050214V-32 | 1813.8 | [M + H]+ | 907.1 |
| 84 | 22651_377 | H-SRAKWNNTLKQIVKK-OH | 8846_HIV-1 Con B Env_084 | 050214V-33 | 1814.9 | [M + H]+ | 907.7 |
| 85 | 22651_378 | H-WNNTLKQIVKKLREQ-OH | 8847_HIV-1 Con B Env_085 | 050214V-34 | — | — | 949.7 |
| 86 | 22651_379 | H-LKQIVKKLREQFGNK-OH | 8848_HIV-1 Con B Env_086 | 050214V-35 | 1829.0 | [M + H]+ | 915.6 |
| 87 | 22651_380 | H-VKKLREQFGNKTIVF-OH | 8849_HIV-1 Con B Env_087 | 050214V-36 | 1806.9 | [M + H]+ | 904.2 |
| 88 | 22651_381 | H-REQFGNKTIVFNQSS-OH | 8850_HIV-1 Con B Env_088 | 050214V-37 | 1754.8 | [M + H]+ | 878.0 |
| 89 | 22651_382 | H-GNKTIVFNQSSGGDP-OH | 8851_HIV-1 Con B Env_089 | 050214V-38 | 1520.6 | [M + H]+ | 761.0 |
| 90 | 22651_383 | H-IVFNQSSGGDPEIVM-OH | 8852_HIV-1 Con B Env_090 | 050214V-39 | 1593.6 | [M + H]+ | 797.0 |
| 91 | 22651_384 | H-QSSGGDPEIVMHSFN-OH | 8853_HIV-1 Con B Env_091 | 050214V-40 | 1606.5 | [M + H]+ | 803.0 |
| 92 | 22651_385 | H-GDPEIVMHSFNCGGE-OH | 8854_HIV-1 Con B Env_092 | 050214V-41 | 1593.5 | [M + H]+ | 796.5 |
| 93 | 22651_386 | H-IVMHSFNCGGEFFYC-OH | 8855_HIV-1 Con B Env_093 | 050214V-42 | 1756.6 | [M + H]+ | 877.9 |
| 94 | 22651_387 | H-SFNCGGEFFVCNTTQ-OH | 8856_HIV-1 Con B Env_094 | 050214V-43 | 1717.6 | [M + H]+ | 859.5 |
| 95 | 22651_388 | H-GGEFFYCNTTQLFNS-OH | 8857_HIV-1 Con B Env_095 | 050214V-44 | 1729.6 | [M + H]+ | 864.5 |
| 96 | 22651_389 | H-FYCNTTQLFNSTWNV-OH | 8858_HIV-1 Con B Env_096 | 050214V-45 | 1838.7 | [M + H]+ | 919.6 |
| 97 | 22651_390 | H-TTQLFNSTWNVNGTW-OH | 8859_HIV-1 Con B Env_097 | 050214V-46 | 1769.9 | [M + H]+ | 885.1 |
| 98 | 22651_391 | H-FNSTWNVNGTWNNNT-OH | 8860_HIV-1 Con B Env_098 | 050214V-47 | 1769.7 | [M + H]+ | 885.5 |
| 99 | 22651_392 | H-WNVNGTWNNNTEGND-OH | 8861_HIV-1 Con B Env_099 | 050214V-48 | 1734.6 | [M + H]+ | 868.0 |
| 100 | 22651_393 | H-GTWNNNTEGNDTITL-OH | 8862_HIV-1 Con B Env_100 | 060214Y-18 | 1651.6 | [M + H]+ | 825.5 |
| 101 | 22651_394 | H-NNTEGNDTITLPCRI-OH | 8863_HIV-1 Con B Env_101 | 060214Y-20 | 1662.7 | [M + H]+ | 831.0 |
| 102 | 22651_395 | H-GNDTITLPCRIKQII-OH | 8864_HIV-1 Con B Env_102 | 060214Y-22 | 1686.2 | [M + H]+ | 843.2 |
| 103 | 22651_396 | H-ITLPCRIKQIINMWQ-OH | 8865_HIV-1 Con B Env_103 | 060214C8 | 1858.3 | [M + H]+ | 929.3 |
| 104 | 22651_397 | H-CRIKQIINMWQEVGK-OH | 8866_HIV-1 Con B Env_104 | 060214C10 | 1848.2 | [M + H]+ | 924.0 |
| 105 | 22651_398 | H-QIINMWQEVGKAMYA-OH | 8867_HIV-1 Con B Env_105 | 060214C12 | 1781.7 | [M + H]+ | 891.5 |
| 106 | 22651_399 | H-MWQEVGKAMYAPPIR-OH | 8868_HIV-1 Con B Env_106 | 140214C5 | 1776.8 | [M + H]+ | 889.1 |
| 107 | 22651_400 | H-VGKAMYAPPIRGQIR-OH | 8869_HIV-1 Con B Env_107 | 140214C7 | 1657.3 | [M + H]+ | 829.3 |
| 108 | 22651_401 | H-MYAPPIRGQIRCSSN-OH | 8870_HIV-1 Con B Env_108 | 140214C9 | 1692.7 | [M + H]+ | 847.0 |
| 109 | 22651_402 | H-PIRGQIRCSSNITGL-OH | 8871_HIV-1 Con B Env_109 | 140214C11 | — | — | 808.1 |
| 110 | 22651_403 | H-QIRCSSNITGLLLTR-OH | 8872_HIV-1 Con B Env_110 | 140214B9 | 1675.8 | [M + H]+ | 838.1 |
| 111 | 22651_404 | H-SSNITGLLLTRDGGN-OH | 8373_HIV-1 Con B Env_111 | 110214G-03 | 1518.4 | [M + H]+ | 759.7 |
| 112 | 22651_405 | H-TGLLLTRDGGNNNTN-OH | 8874_HIV-1 Con B Env_112 | 110214G-05 | 1560.7 | [M + H]+ | 780.5 |
| 113 | 22651_406 | H-LTRDGGNNNTNETEI-OH | 8875_HIV-1 Con B Env_113 | 110214G-07 | 1648.6 | [M + H]+ | 824.5 |
| 114 | 22651_407 | H-GGNNNTNETEIFRPG-OH | 8676_HIV-1 Con B Env_114 | 110214G-09 | 1621.6 | [M + H]+ | 810.5 |
| 115 | 22651_408 | H-NTNETEIFRPGGGDM-OH | 8877_HIV-1 Con B Env_115 | 110214G-11 | 1639.6 | [M + H]+ | 819.5 |
| 116 | 22651_409 | H-TEIFRPGGGDMRDNW-OH | 8878_HIV-1 Con B Env_116 | 110214G-13 | — | — | 876.0 |
| 117 | 22651_410 | H-RPGGGDMRDNWRSEL-OH | 8679_HIV-1 Con B Env_117 | 110214G-15 | 1745.9 | [M + H]+ | 873.5 |
| 118 | 22651_411 | H-GDMRDNWRSELYKYK-OH | 8880_HIV-1 Con B Env_118 | 110214G-17 | — | — | 981.1 |
| 119 | 22651_412 | H-DNWRSELYKYKVVKI-OH | 8881_HIV-1 Con B Env_119 | 110214G-19 | — | — | 971.5 |
| 120 | 22651_413 | H-SELYKYKVVKIEPLG-OH | 8882_HIV-1 Con B Env_120 | 110214G-21 | 1767.9 | [M + H]+ | 883.7 |
| 121 | 22651_414 | H-KYKVVKIEPLGVAPT-OH | 8883_HIV-1 Con B Env_121 | 110214G-23 | 1642.8 | [M + H]+ | 821.6 |
| 122 | 22651_415 | H-VKIEPLGVAPTKAKR-OH | 8884_HIV-1 Con B Env_122 | 110214G-25 | 1606.8 | [M + H]+ | 804.1 |
| 123 | 22651_416 | H-PLGVAPTKAKRRVVQ-OH | 8885_HIV-1 Con B Env_123 | 110214V-02 | 1620.9 | [M + H]+ | 810.6 |
| 124 | 22651_417 | H-APTKAKRRVVQREKR-OH | 8886_HIV-1 Con B Env_124 | 110214V-04 | — | — | 912.2 |
| 125 | 22651_418 | H-AKRRVVQREKRAVGI-OH | 8887_HIV-1 Con B Env_125 | 110214V-06 | — | — | 883.7 |
| 126 | 22651_419 | H-VVQREKRAVGIGAMF-OH | 8888_HIV-1 Con B Env_126 | 110214V-08 | 1662.8 | [M + H]+ | 831.1 |
| 127 | 22651_420 | H-EKRAMGLGAMFLGFL-OH | 8889_HIV-1 Con B Env_127 | 110214V-10 | 1610.7 | [M + H]+ | 805.1 |
| 128 | 22651_421 | H-VGIGAMFLGFLGAAG-OH | 8890_HIV-1 Con B Env_128 | 110214V-12 | 1380.7 | [M + H]+ | 891.0 |
| 129 | 22651_422 | H-AMFLGFLGAAGSTMG-OH | 8891_HIV-1 Con B Env_129 | 110214V-14 | 1480.9 | [M + H]+ | 718.5 |
| 130 | 22651_423 | H-GFLGAAGEIMGAASM-OH | 8892_HIV-1 Con B Env_130 | 110214V-16 | 1326.4 | [M + H]+ | 565.0 |
| 131 | 22651_424 | H-AAGSTMGAASMTLTV-OH | 8893_HIV-1 Con B Env_131 | 110214V-15 | 1368.5 | [M + H]+ | 635.0 |
| 132 | 22651_425 | H-TMGAASMTLIVQARQ-OH | 8894_HIV-1 Con B Env_132 | 110214V-20 | 1565.7 | [M + H]+ | 783.5 |
| 133 | 22651_426 | H-ASMTLTVOARQLLSG-OH | 8895_HIV-1 Con B Env_133 | 110214V-22 | 1576.5 | [M + H]+ | 788.8 |
| 134 | 22651_427 | H-LTVQARQLLSGIVQQ-OH | 8896_HIV-1 Con B Env_134 | 110214V-24 | 1655.7 | [M + H]+ | 817.6 |
| 135 | 22651_428 | H-ARQLLSGIVQGQNNL-OH | 8897_HIV-1 Con B Env_135 | 110214V-25 | 1631.8 | [M + H]+ | 841.6 |
| 136 | 22651_429 | H-LSGIVQQQNNLLRAI-OH | 8898_HIV-1 Con B Env_136 | 110214V-25 | 1666.3 | [M + H]+ | 834.1 |
| 137 | 22651_430 | H-VQQQNNLLRAIEAQQ-OH | 8899_HIV-1 Con B Env_137 | 110214V-30 | 1754.6 | [M + H]+ | 827.5 |
| 138 | 22651_431 | H-NNLLRAIEAQQHLIQ-OH | 8900_HIV-1 Con B Env_138 | 110214V-32 | 1762.8 | [M + H]+ | 851.1 |
| 139 | 22651_432 | H-RAIEAQQHLLQLTVW-OH | 8901_HIV-1 Con B Env_139 | 110214V-34 | 1807.1 | [M + H]+ | 903.7 |
| 140 | 22651_433 | H-AQQHLLQLTVWGIKQ-OH | 8902_HIV-1 Con B Env_140 | 110214V-36 | 1763.8 | [M + H]+ | 882.1 |
| 141 | 22651_434 | H-LLQLTVWGIKQLQAR-OH | 8903_HIV-1 Con B Env_141 | 110214V-35 | 1768.2 | [M + H]+ | 834.2 |
| 142 | 22651_435 | H-TVWGIKCLQARVLAV-OH | 8904_HIV-1 Con B Env_142 | 110214V-40 | 1682.9 | [M + H]+ | 341.2 |
| 143 | 22651_436 | H-IKQLOARVLAVERYL-OH | 8905_HIV-1 Con B Env_143 | 110214V-42 | 1800.0 | [M + H]+ | 900.7 |
| 144 | 22651_437 | H-QARVIAVERVLKQQQ-OH | 8906_HIV-1 Con B Env_144 | 110214V-44 | — | — | 909.2 |
| 145 | 22651_438 | H-RYVERYLKDQQLLGI-OH | 8907_HIV-1 Con B Env_145 | 110214V-46 | 1760.7 | [M + H]+ | 890.2 |
| 146 | 22651_439 | H-RYVERYLKDQQLLGI-OH | 8908_HIV-1 Con B Env_146 | 110214V-48 | 1780.2 | [M + H]+ | 891.1 |
| 147 | 22651_440 | H-OQQLLGFWGCSGKLI-OH | 8909_HIV-1 Con B Env_147 | 180214V-02 | 1631.7 | [M + H]+ | 816.0 |
| 148 | 22651_441 | H-LGIWGCSGKLICTIT-OH | 8910_HIV-1 Con B Env_148 | 180214V-04 | 1553.3 | [M + H]+ | 777.1 |
| 149 | 22651_442 | H-GCSGKLKIITTTVPWN-OH | 8911_HIV-1 Con B Env_149 | 180214V-06 | 1580.7 | [M + H]+ | 790.5 |
| 150 | 22651_443 | H-KLICTTTVPWNASWS-OH | 8912_HIV-1 Con B Env_150 | 180214V-08 | 1707.7 | [M + H]+ | 854.0 |
| 151 | 22651_444 | H-TITVPWNASWSNKSL-OH | 8913_HIV-1 Con B Env_151 | 180214V-10 | 1592.7 | [M + H]+ | 845.5 |
| 152 | 22651_445 | H-PWNASWSNKSLDDW-OH | 8914_HIV-1 Con B Env_152 | 180214V-12 | 1633.1 | [M + H]+ | 917.5 |
| 153 | 22651_446 | H-SWSNKSLDEIWDNMT-OH | 8915_HIV-1 Con B Env_153 | 180214V-14 | 1826.7 | [M + H]+ | 914.0 |
| 154 | 22651_447 | H-KSLDEIWDNMTWMEW-OH | 8916_HIV-1 Con B Env_154 | 180214V-16 | 1986.0 | [M + H]+ | 983.0 |
| 155 | 22651_448 | H-EIWDNMTWMIWEREI-OH | 8917_HIV-1 Con B Env_155 | 180214V-18 | 2069.8 | [M + H]+ | 1035.1 |
| 156 | 22651_449 | H-HMTWMEWEREIDNVT-OH | 8918_HIV-1 Con B Env_156 | 180214V-20 | — | — | 1009.3 |
| 157 | 22651_450 | H-MEWEREIDNYISLSY-OH | 8919_HIV-1 Con B Env_157 | 180214V-27 | 1962.8 | [M + H]+ | 981.3 |
| 158 | 22651_451 | H-REIDNYTSLSNTBE-OH | 8920_HIV-1 Con B Env_158 | 180214V-24 | 1842.9 | [M + H]+ | 922.1 |
| 159 | 22651_452 | H-NYISUYTUEESQN-OH | 8921_HIV-1 Con B Env_159 | 180214V-28 | 1768.3 | [M + H]+ | 894.6 |
| 160 | 22651_453 | H-LNTUEESQNQQEK-OH | 8922_HIV-1 Con B Env_160 | 180214V-28 | 1636.8 | [M + H]+ | 915.6 |
| 161 | 22651_454 | H-LIEESQNQQEBNEQE-OH | 8923_HIV-1 Con B Env_161 | 180214V-30 | 1847.7 | [M + H]+ | 923.3 |
| 162 | 22651_455 | H-SQNQQEKNEQQIEL-OH | 8924_HIV-1 Con B Env_162 | 180214V-32 | 1829.8 | [M + H]+ | 915.6 |
| 163 | 22651_456 | H-QEKNEQELLELOKWA-OH | 8925_HIV-1 Con B Env_163 | 180214V-34 | 1871.9 | [M + H]+ | 937.2 |
| 164 | 22651_457 | H-EQELLELDKWASLWN-OH | 8926_HIV-1 Con B Env_164 | 180214V-38 | 1874.6 | [M + H]+ | 937.8 |
| 165 | 22651_458 | H-ELDKWASIWNWFDI-OH | 8927_HIV-1 Con B Env_165 | 180214V-38 | 3937.3 | [M + H]+ | 969.2 |
| 166 | 22651_459 | H-LWASLWNWFWTNWL-OH | 8928_HIV-1 Con B Env_166 | 180214V-40 | 1981.3 | [M + H]+ | 991.0 |
| 167 | 22651_460 | H-LWMWFOMNWDAYIK-OH | 8929_HIV-1 Con B Env_167 | 180214V-42 | 2100.8 | [M + H]+ | 1050.1 |
| 168 | 22651_461 | H-DITNWLWYIKIRM-OH | 8930_HIV-1 Con B Env_168 | 180214V-44 | 2000.8 | [M + H]+ | 1002.5 |
| 169 | 22651_462 | H-NWLWMKAMNGG-OH | 8931_HIV-1 Con B Env_169 | 180214V-48 | 1855.30 | [M + H]+ | — |
| 170 | 22651_463 | H-VIKIFIMIMVGGLIG-OH | 8932_HIV-1 Con B Env_170 | 180214V-48 | 1649.9 | [M + H]+ | 825.7 |
| 171 | 22651_464 | H-FIMIVGGLIGIRIVF-OH | 8833_HIV-1 Con B Env_171 | 130214Z-02 | 1648.9 | [M + H]+ | 824.5 |
| 172 | 22651_465 | H-VGGLIGLRIVFAVLS-OH | 8834_HIV-1 Con B Env_172 | 130214Z-04 | 1514.8 | [M + H]+ | 737.6 |
| 173 | 22651_466 | H-IGLRIVFAVLSIVNR-OH | 8835_HIV-1 Con B Env_173 | 130214Z-06 | 3671.3 | [M + H]+ | 835.8 |
| 174 | 22651_467 | H-IVFAVLSIVNRYRGG-OH | 8836_HIV-1 Con B Env_174 | 130214Z-03 | 1672.8 | [M + H]+ | 836.2 |
| 175 | 22651_468 | H-VLSIVNRVRQGYSPI-OH | 8837_HIV-1 Con B Env_175 | 130214Z-10 | 1700.8 | [M + H]+ | 851.1 |
| 176 | 22651_469 | H-VNRYFQGYSPLSFQT-OH | 8838_HIV-1 Con B Env_176 | 130214Z-12 | 1751.8 | [M + H]+ | 876.6 |
| 177 | 22651_470 | H-RQGYSPLSFQTRLPA-OH | 8839_HIV-1 Con B Env_177 | 130214Z-14 | 1722.2 | [M + H]+ | 861.3 |
| 178 | 22651_471 | H-SPLSFQTRIPAPRGP-OH | 8840_HIV-1 Con B Env_178 | 170214Y-04 | 1625.7 | [M + H]+ | 8126.6 |
| 179 | 22651_472 | H-FQTRLPAPRGPDRPE-OH | 8841_HIV-1 Con B Env_179 | 170214Y-05 | 1739.2 | [M + H]+ | 869.3 |
| 180 | 22651_473 | H-LPAPRGPORPEGIEE-OH | 8842_HIV-1 Con B Env_180 | 170214Y-08 | 1632.8 | [M + H]+ | 817.0 |
| 181 | 22651_474 | H-RGPDEPEGIEEGGGE-OH | 8843_HIV-1 Con B Env_181 | 170214Y-10 | 1627.5 | [M + H]+ | 814.0 |
| 182 | 22651_475 | H-RPEGIEEEGGERDRD-OH | 8844_HIV-1 Con B Env_182 | 170214Y-12 | — | — | 872.5 |
| 183 | 22651_476 | H-IEEEGGERDRDSSGR-OH | 8845_HIV-1 Con B Env_183 | 170214Y-14 | — | — | 881.0 |
| 184 | 22651_477 | H-GGERDSDRSGRIVDG-OH | 8846_HIV-1 Con B Env_184 | 170214Y-15 | 1649.1 | [M + H]+ | 878.0 |
| 185 | 22651_478 | H-DRDRSGRLVDGFLAL-OH | 8847_HIV-1 Con B Env_185 | 170214Y-18 | 1690.3 | [M + H]+ | 845.7 |
| 186 | 22651_479 | H-SGRLVDGFIAIIWDD-OH | 8848_HIV-1 Con B Env_186 | 170214Y-20 | 1678.7 | [M + H]+ | 839.0 |
| 187 | 22651_480 | H-VDGFLALIWDDLASL-OH | 8849_HIV-1 Con B Env_187 | 210214R-03 | 1734.8 | [M + H]+ | 867.1 |
| 188 | 22651_481 | H-LALIWDDLRSLCLFS-OH | 8850_HIV-1 Con B Env_188 | 210214R-05 | 1765.4 | [M + H]+ | 883.3 |
| 189 | 22651_482 | H-WDDLASLCLFSYHRL-OH | 8851_HIV-1 Con B Env_189 | 210214R-07 | 1926.2 | [M + H]+ | 963.1 |
| 190 | 22651_483 | H-RSLCLFSYHRLRDIL-OH | 8852_HIV-1 Con B Env_190 | 210214R-08 | — | — | 947.0 |
| 191 | 22651_484 | H-LFSYERLRDLILIVT-OH | 8853_HIV-1 Con B Env_191 | 210214R-11 | 1859.9 | [M + H]+ | 830.2 |
| 192 | 22651_485 | H-HRLRQLLLIVERIVE-OH | 8854_HIV-1 Con B Env_192 | 210214R-13 | — | — | 922.7 |
| 193 | 22651_486 | H-DLLLIVYARVELLGR-OH | 8855_HIV-1 Con B Env_193 | 210214R-15 | 1725.4 | [M + H]+ | 862.4 |
| 194 | 22651_487 | H-IVTRIVELLGREGWE-OH | 8856_HIV-1 Con B Env_194 | 210214R-17 | — | — | 839.2 |
| 195 | 22651_488 | H-IVELLGRRGWEVLKY-OH | 8857_HIV-1 Con B Env_195 | 210214R-19 | 1632.9 | [M + H]+ | 916.2 |
| 196 | 22651_489 | H-LGRRGWEVLKYWWNL-OH | 8858_HIV-1 Con B Env_196 | 210214R-21 | 1977.0 | [M + H]+ | 938.7 |
| 197 | 22651_490 | H-GWEVLKVWWMLLQYW-OH | 8859_HIV-1 Con B Env_197 | 210214R-23 | — | — | 1043.1 |
| 198 | 22651_491 | H-LKYWWNLLQVWSQEL-OH | 8860_HIV-1 Con B Env_198 | 210214R-25 | 2072.2 | [M + H]+ | 1036.2 |
| 199 | 22651_492 | H-WNLLQVWSQELKNSA-OH | 8861_HIV-1 Con B Env_199 | 210214R-27 | 1881.8 | [M + H]+ | 941.0 |
| 200 | 22651_493 | H-QYWSQELKNSAVSLL-OH | 8862_HIV-1 Con B Env_200 | 210214R-29 | 1767.8 | [M + H]+ | 384.0 |
| 201 | 22651_494 | H-QELKNSAVSLINATA-OH | 8863_HIV-1 Con B Env_201 | 210214R-31 | 1559.3 | [M + H]+ | 780.2 |
| 202 | 22651_495 | H-NSAVSLLNATAIAVA-OH | 8864_HIV-1 Con B Env_202 | 210214R-33 | 1414.2 | [M + H]+ | 708.1 |
| 203 | 22651_496 | H-SLINATAIAVAGGTD-OH | 8865_HIV-1 Con B Env_203 | 210214R-35 | 1446.2 | [M + H]+ | 724.0 |
| 204 | 22651_497 | H-ATAIAVAEGTDRVIE-OH | 8866_HIV-1 Con B Env_204 | 210214R-37 | 1515.7 | [M + H]+ | 758.5 |
| 205 | 22651_498 | H-AVAEGTDRVIEVVQR-OH | 8867_HIV-1 Con B Env_205 | 210214R-38 | 1641.8 | [M + H]+ | 821.6 |
| 206 | 22651_499 | H-GTDRVIEVVQFACRA-OH | 8868_HIV-1 Con B Env_206 | 210214R-41 | 1673.3 | [M + H]+ | 837.2 |
| 207 | 22651_500 | H-VIEVVQRACRAELHI-OH | 8869_HIV-1 Con B Env_207 | 210214R-43 | 1720.4 | [M + H]+ | 850.8 |
| 208 | 22651_501 | H-VQRACRAILHIFRFI-OH | 8870_HIV-1 Con B Env_208 | 210214R-45 | — | — | 901.7 |
| 209 | 22651_502 | H-CRAILHIPRRIRQGL-OH | 8871_HIV-1 Con B Env_209 | 210214R-47 | — | — | 901.7 |
| 210 | 22651_503 | H-LHIPRRIRQGLERAL-OH | 8872_HIV-1 Con B Env_210 | 210214Z-02 | — | — | 914.7 |
| 211 | 22651_504 | H-RRIFQGLERALL-OH | 8373_HIV-1 Con B Env_211 | 19021482 | — | — | 741.1 |

| | | | | Theor. | | Exp. MW | | | | |
|---|---|---|---|---|---|---|---|---|---|---|
| | | MW (3) | | MW | Theor. | vs. | | | | |
| | MW (2) | detected | MW (3) | (average) | MW + TFA | Theor. | | Amount | | |
| | label | [g/mol] | label | [g/mol] | [g/mol] | MW | Purity [%] | [mg] | Purified | Comment |
| 1 | [M + 2H]2+ | 662.8 | [M + 3H]3+ | 1985.38 | 2783.38 | pass | 85.3 | 65.0 | Y | |
| 2 | [M + 2H]2+ | 649.7 | [M + 3H]3+ | 1946.25 | 2516.25 | pass | 87.8 | 65.0 | Y | |
| 3 | [M + 2H]2+ | — | — | 1906.24 | 2248.24 | pass | 87.1 | 65.0 | Y | |
| 4 | [M + 2H]2+ | — | — | 1824.34 | 2052.34 | pass | 80.2 | 66.0 | Y | |
| 5 | [M + 2H]2+ | 1584.37 | [M + 2Na]2+ | 1540.92 | 1654.92 | pass | approx. 80 | 65.0 | N | 2) |
| 6 | [M + 2H]2+ | 555.8 | [M + 3H]3+ | 1665.09 | 1893.09 | pass | 90.0 | 65.0 | Y | |
| 7 | [M + 2H]2+ | 1777.56 | [M + H]+ | 1777.12 | 2005.12 | pass | approx. 80 | 65.0 | N | 1) |
| 8 | [M + 2H]2+ | 565.8 | [M + 3H]3+ | 1694.98 | 1922.98 | pass | 94.7 | 66.0 | Y | |
| 9 | [M + 2H]2+ | 604.2 | [M + 3H]3+ | 1810.11 | 2038.11 | pass | 87.2 | 67.0 | Y | |
| 10 | [M + 2H]2+ | 576.4 | [M + 3H]3+ | 1726.97 | 1954.97 | pass | 88.8 | 67.0 | Y | |
| 11 | [M + 2H]2+ | — | — | 1652.91 | 1880.91 | pass | 85.9 | 67.0 | Y | |
| 12 | [M + 2H]2+ | 519.8 | [M + 3H]3+ | 1556.74 | 1898.74 | pass | 87.0 | 66.0 | Y | |
| 13 | [M + 2H]2+ | — | — | 1635.76 | 1863.76 | pass | 84.7 | 66.0 | Y | |
| 14 | [M + 2H]2+ | 541.9 | [M + 3H]3+ | 1622.72 | 1964.72 | pass | 89.4 | 67.0 | Y | |
| 15 | [M + 2H]2+ | 581.7 | [M + 3H]3+ | 1741.86 | 2197.86 | pass | 85.3 | 66.0 | Y | |
| 16 | [M + 2H]2+ | — | — | 1678.82 | 2020.82 | pass | 82.3 | 65.0 | Y | |
| 17 | [M + 2H]2+ | 555.0 | [M + 3H]3+ | 1661.80 | 2003.80 | pass | 88.0 | 66.0 | Y | |
| 18 | [M + 2H]2+ | 527.2 | [M + 3H]3+ | 1578.71 | 1806.71 | pass | 93.9 | 66.0 | Y | |
| 19 | [M + 2H]2+ | — | — | 1653.81 | 1653.81 | pass | 91.1 | 66.0 | Y | |
| 20 | [M + 2H]2+ | 566.5 | [M + 3H]3+ | 1696.76 | 1696.76 | pass | 86.0 | 66.0 | Y | |
| 21 | [M + 2H]2+ | — | — | 1852.01 | 1852.01 | pass | 80.5 | 66.0 | Y | 5) |
| 22 | [M + 2H]2+ | 628.9 | [M + 3H]3+ | 1884.07 | 1884.07 | pass | 93.6 | 66.0 | Y | |
| 23 | [M + 2H]2+ | 639.5 | [M + 3H]3+ | 1916.13 | 1916.13 | pass | 95.5 | 65.0 | Y | |
| 24 | [M + 2H]2+ | 640.5 | [M + 3H]3+ | 1919.14 | 1919.14 | pass | 94.8 | 65.0 | Y | |
| 25 | [M + 2H]2+ | 620.5 | [M + 3H]3+ | 1859.08 | 1859.08 | pass | 85.9 | 65.0 | Y | |
| 26 | [M + 2H]2+ | 615.5 | [M + 3H]3+ | 1844.02 | 1844.02 | pass | 82.9 | 67.0 | Y | |
| 27 | [M + 2H]2+ | 582.9 | [M + 3H]3+ | 1746.01 | 1746.01 | pass | 89.3 | 65.0 | Y | |
| 28 | [M + 2H]2+ | 572.6 | [M + 3H]3+ | 1715.04 | 2057.04 | pass | 87.1 | 65.0 | Y | |
| 29 | [M + 2H]2+ | 544.2 | [M + 3H]3+ | 1630.03 | 1972.03 | pass | 88.1 | 66.0 | Y | |
| 30 | [M + 2H]2+ | 535.7 | [M + 3H]3+ | 1604.99 | 1832.99 | pass | 88.2 | 66.0 | Y | |
| 31 | [M + 2H]2+ | — | — | 1650.97 | 1764.97 | pass | 82.4 | 65.0 | Y | |
| 32 | [M + 2H]2+ | — | — | 1613.81 | 1727.81 | pass | 81.9 | 66.0 | Y | |
| 33 | [M + 2H]2+ | — | — | 1586.64 | 1700.64 | pass | 80.4 | 66.0 | Y | |
| 34 | [M + 2H]2+ | — | — | 1554.62 | 1782.62 | pass | 84.6 | 65.0 | Y | |
| 35 | [M + 2H]2+ | 524.5 | [M + 3H]3+ | 1570.61 | 1912.61 | pass | 84.4 | 65.0 | Y | 3) |
| 36 | [M + 2H]2+ | 546.7 | [M + 3H]3+ | 1637.75 | 2093.75 | pass | 85.7 | 65.0 | Y | |
| 37 | [M + 2H]2+ | 572.2 | [M + 3H]3+ | 1713.91 | 2169.91 | pass | 87.7 | 65.0 | Y | |
| 38 | [M + 2H]2+ | 557.8 | [M + 3H]3+ | 1670.83 | 2012.83 | pass | 95.0 | 66.0 | Y | |
| 39 | [M + 2H]2+ | 580.8 | [M + 3H]3+ | 1740.00 | 2196.00 | pass | 89.3 | 67.0 | Y | |
| 40 | [M + 2H]2+ | 589.6 | [M + 3H]3+ | 1765.97 | 2221.97 | pass | 81.9 | 66.0 | Y | |
| 41 | [M + 2H]2+ | 600.5 | [M + 3H]3+ | 1799.05 | 2255.05 | pass | 90.5 | 65.0 | Y | |
| 42 | [M + 2H]2+ | 639.7 | [M + 3H]3+ | 1916.21 | 2486.21 | pass | 91.6 | 65.0 | Y | |
| 43 | [M + 2H]2+ | 609.5 | [M + 3H]3+ | 1826.17 | 2168.17 | pass | 95.2 | 66.0 | Y | |
| 44 | [M + 2H]2+ | — | — | 1735.96 | 1963.96 | pass | 86.0 | 65.0 | Y | |
| 45 | [M + 2H]2+ | — | — | 1679.80 | 1907.80 | pass | 86.9 | 65.0 | Y | |
| 46 | [M + 2H]2+ | 565.5 | [M + 3H]3+ | 1693.84 | 1921.84 | pass | 85.4 | 65.0 | Y | |
| 47 | [M + 2H]2+ | — | — | 1674.76 | 1902.76 | pass | 85.3 | 65.0 | Y | |
| 48 | [M + 2H]2+ | 558.1 | [M + 3H]3+ | 1671.89 | 1899.89 | pass | 88.0 | 65.0 | Y | |
| 49 | [M + 2H]2+ | 526.9 | [M + 3H]3+ | 1577.87 | 1805.87 | pass | 91.2 | 67.0 | Y | |
| 50 | [M + 2H]2+ | 542.3 | [M + 3H]3+ | 1623.89 | 1851.83 | pass | 85.5 | 65.0 | Y | |
| 51 | [M + 2H]2+ | 548.5 | [M + 3H]3+ | 1642.98 | 1870.98 | pass | 93.8 | 65.0 | Y | |
| 52 | [M + 2H]2+ | 568.8 | [M + 3H]3+ | 1704.05 | 2046.05 | pass | 90.5 | 66.0 | Y | |
| 53 | [M + 2H]2+ | — | — | 1648.92 | 1876.92 | pass | 87.6 | 66.0 | Y | |
| 54 | [M + 2H]2+ | — | — | 1614.00 | 1956.00 | pass | 84.0 | 66.0 | Y | |
| 55 | [M + 2H]2+ | 538.8 | [M + 3H]3+ | 1613.92 | 1955.92 | pass | 91.7 | 67.0 | Y | |
| 56 | [M + 2H]2+ | — | — | 1625.90 | 2081.90 | pass | 92.6 | 66.0 | Y | |
| 57 | [M + 2H]2+ | 546.8 | [M + 3H]3+ | 1637.92 | 2093.92 | pass | 89.1 | 66.0 | Y | |
| 58 | [M + 2H]2+ | 528.2 | [M + 3H]3+ | 1581.70 | 1923.70 | pass | 89.2 | 66.0 | Y | |
| 59 | [M + 2H]2+ | — | — | 1527.67 | 1641.67 | pass | 81.6 | 66.0 | Y | 3) |
| 60 | [M + 2H]2+ | — | — | 1516.69 | 1744.69 | pass | 91.2 | 67.0 | Y | |
| 61 | [M + 2H]2+ | 5375 | [M + 3H]3+ | 1609.85 | 1951.85 | pass | 86.9 | 65.0 | Y | |
| 62 | [M + 2H]2+ | — | — | 1637.91 | 1979.91 | pass | 89.3 | 65.0 | Y | |
| 63 | [M + 2H]2+ | 535.5 | [M + 3H]3+ | 1603.88 | 1945.88 | pass | 87.7 | 66.0 | Y | |
| 64 | [M + 2H]2+ | — | — | 1540.77 | 1654.77 | pass | 90.4 | 66.0 | Y | 3) |
| 65 | [M + 2H]2+ | — | — | 1614.79 | 1728.79 | pass | 92.8 | 66.0 | Y | |
| 66 | [M + 2H]2+ | 549.1 | [M + 3H]3+ | 1644.78 | 1872.78 | pass | 94.0 | 66.0 | Y | |
| 67 | [M + 2H]2+ | — | — | 1749.87 | 1977.87 | pass | 93.7 | 65.0 | Y | |
| 68 | [M + 2H]2+ | 574.8 | [M + 3H]3+ | 1721.86 | 2063.86 | pass | 88.0 | 65.0 | Y | |
| 69 | [M + 2H]2+ | 579.2 | [M + 3H]3+ | 1734.91 | 2076.91 | pass | 95.9 | 65.0 | Y | |
| 70 | [M + 2H]2+ | — | — | 1691.88 | 1919.88 | pass | 94.6 | 65.0 | Y | |
| 71 | [M + Na]+ | 1717.22 | [M + 2Na]2+ | 1670.90 | 1898.90 | pass | approx. 80 | 66.0 | Y | 2) |
| 72 | [M + 2H]2+ | 572.5 | [M + 3H]3+ | 1714.94 | 1942.94 | pass | 92.8 | 67.0 | Y | |
| 73 | [M + 2H]2+ | — | — | 1704.76 | 1932.76 | pass | 87.3 | 67.0 | Y | |
| 74 | [M + 2H]2+ | 587.5 | [M + 3H]3+ | 1759.94 | 2215.94 | pass | 81.6 | 68.0 | Y | |
| 75 | [M + 2H]2+ | 569.2 | [M + 3H]3+ | 1704.90 | 2274.90 | pass | 85.9 | 72.0 | Y | |
| 76 | [M + 2H]2+ | 556.9 | [M + 3H]3+ | 1667.89 | 2237.89 | pass | 93.2 | 74.0 | Y | |
| 77 | [M + 2H]2+ | 535.8 | [M + 3H]3+ | 1604.83 | 2060.83 | pass | 87.0 | 74.0 | Y | |
| 78 | [M + 2H]2+ | — | — | 1551.76 | 1779.76 | pass | 86.9 | 73.0 | Y | |
| 79 | [M + 2H]2+ | 580.9 | [M + 3H]3+ | 1739.95 | 2081.95 | pass | 84.7 | 70.0 | Y | |
| 80 | [M + 2H]2+ | 543.5 | [M + 3H]3+ | 1627.78 | 1969.78 | pass | 84.5 | 69.0 | Y | |
| 81 | [M + 2H]2+ | 556.7 | [M + 3H]3+ | 1666.93 | 2122.93 | pass | 91.1 | 67.0 | Y | |
| 82 | [M + 2H]2+ | 604.6 | [M + 3H]3+ | 1811.05 | 2381.05 | pass | 92.7 | 67.0 | Y | |
| 83 | [M + 2H]2+ | 605.3 | [M + 3H]3+ | 1813.05 | 2383.05 | pass | 85.4 | 65.0 | Y | |
| 84 | [M + 2H]2+ | 605.7 | [M + 3H]3+ | 1814.14 | 2498.14 | pass | 96.0 | 65.0 | Y | |
| 85 | [M + 2H]2+ | 633.6 | [M + 3H]3+ | 1898.21 | 2468.21 | pass | 96.9 | 65.0 | Y | |
| 86 | [M + 2H]2+ | 610.7 | [M + 3H]3+ | 1829.20 | 2513.20 | pass | 92.9 | 65.0 | Y | |
| 87 | [M + 2H]2+ | 603.3 | [M + 3H]3+ | 1807.15 | 2377.15 | pass | 89.6 | 65.0 | Y | |
| 88 | [M + 2H]2+ | 585.8 | [M + 3H]3+ | 1754.91 | 2096.91 | pass | 89.3 | 65.0 | Y | |
| 89 | [M + 2H]2+ | — | — | 1520.61 | 1748.61 | pass | 91.4 | 66.0 | Y | |
| 90 | [M + 2H]2+ | — | — | 1592.78 | 1706.78 | pass | 93.6 | 66.0 | Y | |
| 91 | [M + 2H]2+ | — | — | 1604.71 | 1832.71 | pass | 88.3 | 65.0 | Y | |
| 92 | [M + 2H]2+ | — | — | 1591.72 | 1819.72 | pass | 82.7 | 65.0 | Y | |
| 93 | [M + 2H]2+ | — | — | 1754.03 | 1982.03 | pass | 81.6 | 66.0 | Y | |
| 94 | [M + 2H]2+ | 1718.10 | [M + 3H]3+ | 1717.34 | 1831.84 | pass | approx. 80 | 69.0 | Y | 1) |
| 95 | [M + 2H]2+ | — | — | 1727.86 | 1841.86 | pass | 92.3 | 67.0 | Y | |
| 96 | [M + 2H]2+ | — | — | 1838.01 | 1952.01 | pass | 89.8 | 65.0 | Y | |
| 97 | [M + 2H]2+ | — | — | 1768.87 | 1882.87 | pass | 87.9 | 66.0 | Y | |
| 98 | [M + 2H]2+ | — | — | 1768.78 | 1882.78 | pass | 85.7 | 65.0 | Y | |
| 99 | [M + 2H]2+ | — | — | 1734.67 | 1848.67 | pass | 92.1 | 66.0 | Y | |
| 100 | [M + 2H]2+ | — | — | 1649.65 | 1763.65 | pass | 87.0 | 66.0 | Y | |
| 101 | [M + 2H]2+ | 554.5 | [M + 3H]3+ | 1660.80 | 1888.80 | pass | 84.9 | 65.0 | Y | |
| 102 | [M + 2H]2+ | — | — | 1685.01 | 2027.01 | pass | 86.3 | 6.0 | Y | |
| 103 | [M + 2H]2+ | 620.0 | [M + 3H]3+ | 1857.30 | 2199.30 | pass | 82.5 | 66.0 | Y | |
| 104 | [M + 2H]2+ | 616.3 | [M + 3H]3+ | 1846.22 | 2302.22 | pass | 81.0 | 67.0 | Y | |
| 105 | [M + 2H]2+ | 594.8 | [M + 3H]3+ | 1782.09 | 2010.09 | pass | 85.7 | 66.0 | Y | |
| 106 | [M + 2H]2+ | 593.2 | [M + 3H]3+ | 1777.13 | 2119.13 | pass | 93.3 | 66.0 | Y | |
| 107 | [M + 2H]2+ | 553.3 | [M + 3H]3+ | 1657.02 | 2113.02 | pass | 95.9 | 66.0 | Y | |
| 108 | [M + 2H]2+ | 565.2 | [M + 3H]3+ | 1692.99 | 2034.99 | pass | 89.1 | 65.0 | Y | |
| 109 | [M + 2H]2+ | 539.2 | [M + 3H]3+ | 1614.89 | 1956.89 | pass | 88.7 | 67.0 | Y | |
| 110 | [M + 2H]2+ | 559.3 | [M + 3H]3+ | 1674.98 | 2016.98 | pass | 80.8 | 67.0 | Y | |
| 111 | [M + 2H]2+ | — | — | 1517.65 | 1745.65 | pass | 91.2 | 67.0 | Y | 3) |
| 112 | [M + 2H]2+ | — | — | 1559.63 | 1787.63 | pass | 90.7 | 65.0 | Y | |
| 113 | [M + 2H]2+ | 550.2 | [M + 3H]3+ | 1647.64 | 1875.64 | pass | 92.7 | 66.0 | Y | |
| 114 | [M + 2H]2+ | — | — | 1619.64 | 1847.64 | pass | 85.3 | 65.0 | Y | |
| 115 | [M + 2H]2+ | — | — | 1637.72 | 1865.72 | pass | 91.6 | 65.0 | Y | |
| 116 | [M + 2H]2+ | 584.5 | [M + 3H]3+ | 1750.90 | 2092.90 | pass | 83.5 | 66.0 | Y | |
| 117 | [M + 2H]2+ | 582.5 | [M + 3H]3+ | 1745.89 | 2201.89 | pass | 84.7 | 70.0 | Y | |
| 118 | [M + 2H]2+ | 654.7 | [M + 3H]3+ | 1961.18 | 2531.18 | pass | 89.7 | 66.0 | Y | |
| 119 | [M + 2H]2+ | 647.9 | [M + 3H]3+ | 1941.25 | 2511.25 | pass | 94.0 | 66.0 | Y | |
| 120 | [M + 2H]2+ | 589.7 | [M + 3H]3+ | 1766.10 | 2222.10 | pass | 97.3 | 65.0 | Y | |
| 121 | [M + 2H]2+ | 548.5 | [M + 3H]3+ | 1642.00 | 2098.00 | pass | 95.7 | 66.0 | Y | |
| 122 | [M + 2H]2+ | 536.6 | [M + 3H]3+ | 1606.96 | 2176.96 | pass | 93.4 | 68.0 | Y | |
| 123 | [M + 2H]2+ | 541.0 | [M + 3H]3+ | 1619.97 | 2189.97 | pass | 92.9 | 67.0 | Y | |
| 124 | [M + 2H]2+ | 608.7 | [M + 3H]3+ | 1823.17 | 2735.17 | pass | 94.2 | 66.0 | Y | |
| 125 | [M + 2H]2+ | 589.5 | [M + 3H]3+ | 1766.12 | 2564.12 | pass | 96.5 | 67.0 | Y | |
| 126 | [M + 2H]2+ | 554.5 | [M + 3H]3+ | 1660.99 | 2116.99 | pass | 97.6 | 67.0 | Y | |
| 127 | [M + 2H ]2+ | 537.2 | [M + 3H]3+ | 1606.96 | 1950.96 | pass | 96.0 | 67.0 | Y | |
| 128 | [M + 2H ]2+ | 1403.00 | [M + 3H]3+ | 1380.67 | 1494.67 | pass | Approx. 80 | 67.0 | Y | 1) |
| 129 | [M + 2H ]2+ | — | — | 1430.70 | 1544.70 | pass | 86.7 | 68.0 | Y | |
| 130 | [M + 2H ]2+ | — | — | 1328.52 | 1442.52 | pass | 91.7 | 67.0 | Y | |
| 131 | [M + 2H ]2+ | — | — | 1366.57 | 1482.57 | pass | 83.6 | 56.0 | Y | 4) |
| 132 | [M + 2H ]2+ | 522.8 | [M + 3H]3+ | 1569.81 | 1793.81 | pass | 93.3 | 67.0 | Y | 3) |
| 133 | [M + 2H ]2+ | — | — | 1575.84 | 1803.84 | pass | 83.3 | 68.0 | Y | 3) |
| 134 | [M + 2H ]2+ | 532.2 | [M + 3H]3+ | 1693.94 | 1881.94 | pass | 87.5 | 67.0 | Y | |
| 135 | [M + 2H ]2+ | 561.6 | [M + 3H]3+ | 1681.91 | 1908.91 | pass | 96.8 | 67.0 | Y | |
| 136 | [M + 2H ]2+ | 556.5 | [M + 3H]3+ | 1666.34 | 1894.94 | pass | 91.7 | 67.0 | Y | |
| 137 | [M + 2H ]2+ | 585.3 | [M + 3H]3+ | 1753.94 | 1930.94 | pass | 97.0 | 66.0 | Y | |
| 138 | [M + 2H ]2+ | 587.8 | [M + 3H]3+ | 1781.01 | 2103.01 | pass | 99.3 | 67.0 | Y | |
| 139 | [M + 2H ]2+ | — | — | 1806.09 | 2148.09 | pass | 89.1 | 69.0 | Y | |
| 140 | [M + 2H ]2+ | 588.5 | [M + 3H]3+ | 1763.06 | 2109.06 | pass | 92.0 | 66.0 | Y | |
| 141 | [M + 2H ]2+ | — | — | 1787.14 | 2103.14 | pass | 90.1 | 66.0 | Y | |
| 142 | [M + 2H ]2+ | 561.8 | [M + 3H]3+ | 1652.03 | 2024.03 | pass | 93.6 | 66.0 | Y | |
| 143 | [M + 2H ]2+ | 601.0 | [M + 3H]3+ | 1806.18 | 2256.18 | pass | 90.7 | 66.0 | Y | |
| 144 | [M + 2H ]2+ | 676.8 | [M + 3H]3+ | 1817.08 | 2273.08 | pass | 94.8 | 69.0 | Y | |
| 145 | [M + 2H ]2+ | 587.2 | [M + 3H]3+ | 1759.08 | 2101.08 | pass | 93.6 | 70.0 | Y | |
| 146 | [M + 2H ]2+ | — | — | 1780.08 | 2322.08 | pass | 91.6 | 70.0 | Y | |
| 147 | [M + 2H ]2+ | 544.5 | [M + 3H]3+ | 1630.92 | 1858.92 | pass | 87.8 | 70.0 | Y | |
| 148 | [M + 2H ]2+ | — | — | 1552.89 | 1780.85 | pass | 82.6 | 73.0 | Y | |
| 149 | [M + 2H ]2+ | — | — | 1579.83 | 1807.83 | pass | 82.4 | 71.0 | Y | |
| 150 | [M + 2H ]2+ | — | — | 1706.96 | 1934.96 | pass | 81.8 | 67.0 | Y | |
| 151 | [M + 2H ]2+ | — | — | 1681.84 | 1913.84 | pass | 85.6 | 67.0 | Y | |
| 152 | [M + 2H ]2+ | — | — | 1832.98 | 2050.98 | pass | 83.0 | 68.0 | Y | |
| 153 | [M + 2H ]2+ | — | — | 1825.95 | 2053.95 | pass | 81.9 | 67.0 | Y | |
| 154 | [M + 2H ]2+ | — | — | 1984.20 | 2222.20 | pass | 84.8 | 68.0 | Y | |
| 155 | [M + 2H ]2+ | — | — | 2068.27 | 2298.27 | pass | 83.9 | 68.0 | Y | |
| 156 | [M + 2H ]2+ | 679.4 | [M + 3H]3+ | 2018.17 | 2246.17 | pass | 81.6 | 66.0 | Y | |
| 157 | [M + 2H ]2+ | 664.8 | [M + 3H]3+ | 1962.15 | 2190.15 | pass | 87.8 | 69.0 | Y | |
| 158 | [M + 2H ]2+ | — | — | 1843.06 | 2071.06 | pass | 92.8 | 67.0 | Y | |
| 159 | [M + 2H ]2+ | — | — | 1787.93 | 1901.93 | pass | 81.4 | 67.0 | Y | |
| 160 | [M + 2H ]2+ | 612.9 | [M + 3H]3+ | 1836.01 | 1054.01 | pass | 85.8 | 69.0 | Y | |
| 161 | [M + 2H ]2+ | 616.3 | [M + 3H]3+ | 1849.86 | 2073.85 | pass | 88.2 | 68.0 | Y | |
| 162 | [M + 2H ]2+ | 610.8 | [M + 3H]3+ | 1829.91 | 2057.91 | pass | 90.9 | 70.0 | Y | |
| 163 | [M + 2H ]2+ | 625.3 | [M + 3H]3+ | 1873.02 | 2216.02 | pass | 93.4 | 68.0 | Y | |
| 164 | [M + 2H ]2+ | — | — | 1874.06 | 2307.06 | pass | 91.1 | 71.0 | Y | |
| 165 | [M + 2H ]2+ | — | — | 1936.19 | 2164.19 | pass | 80.8 | 71.0 | Y | |
| 166 | [M + 2H ]2+ | — | — | 1980.24 | 2205.24 | pass | 89.9 | 71.0 | Y | |
| 167 | [M + 2H ]2+ | 700.5 | [M + 3H]3+ | 2098.42 | 2328.42 | pass | 93.3 | 70.0 | Y | |
| 168 | [M + 2H ]2+ | 660.8 | [M + 3H]3+ | 2002.43 | 2231.42 | pass | 83.2 | 70.0 | Y | |
| 169 | — | — | — | 1853.29 | 2081.29 | pass | approx. 20-80 | 68.0 | Y | 2) |
| 170 | [M + 2H]2+ | 1672.77 | [M + Na]+ | 1650.14 | 1878.14 | pass | approx. 80 | 68.0 | Y | 1) |
| 171 | [M + 2H]2+ | 1648.75 | [M + H]+ | 1648.13 | 1879.13 | pass | approx. 80) | 67.0 | Y | 1) |
| 172 | [M + 2H]2+ | 1514.30 | [M + H]+ | 1513.89 | 1741.89 | pass | approx. 80 | 68.0 | Y | 1) |
| 173 | [M + 2H]2+ | — | — | 1670.08 | 2012.08 | pass | 85.3 | 67.0 | Y | 3) |
| 174 | [M + 2H]2+ | 558.0 | [M + 3H]3+ | 1671.02 | 2013.02 | pass | 95.5 | 66.0 | Y | |
| 175 | [M + 2H]2+ | 567.9 | [M + 3H]3+ | 1701.01 | 2043.01 | pass | 89.8 | 65.0 | Y | |
| 176 | [M + 2H]2+ | 584.8 | [M + 3H]3+ | 1751.97 | 2093.97 | pass | 88.3 | 67.0 | Y | |
| 177 | [M + 2H]2+ | — | — | 1720.97 | 2062.97 | pass | 87.9 | 68.0 | Y | |
| 178 | [M + 2H]2+ | 543.3 | [M + 3H]3+ | 2623.90 | 1985.90 | pass | 95.4 | 68.0 | Y | |
| 179 | [M + 2H]2+ | 550.0 | [M + 3H]3+ | 1736.97 | 2192.97 | pass | 90.0 | 68.0 | Y | |
| 180 | [M + 2H]2+ | 545.2 | [M + 3H]3+ | 1632.80 | 1974.80 | pass | 91.1 | 68.0 | Y | |
| 181 | [M + 2H]2+ | 543.1 | [M + 3H]3+ | 1626.64 | 1965.64 | pass | 88.5 | 69.0 | Y | |
| 182 | [M + 2H]2+ | 562.2 | [M + 3H]3+ | 1743.75 | 2193.75 | pass | 90.4 | 67.0 | Y | |
| 183 | [M + 2H]2+ | 587.8 | [M + 3H]3+ | 1780.79 | 2330.79 | pass | 9085.4 | 67.0 | Y | |
| 184 | [M + 2H]2+ | 549.2 | [M + 3H]3+ | 1646.73 | 2214.73 | pass | 97.1 | 67.0 | Y | |
| 185 | [M + 2H]2+ | 564.3 | [M + 3H]3+ | 1689.91 | 7145.91 | pass | 89.2 | 67.0 | Y | |
| 186 | [M + 2H]2+ | — | — | 1676.90 | 1904.90 | pass | 85.0 | 68.0 | Y | |
| 187 | [M + 2H]2+ | 578.5 | [M + 3H]3+ | 1743.04 | 1961.01 | pass | 88.9 | 68.0 | Y | |
| 188 | [M + 2H]2+ | — | — | 1765.12 | 1993.12 | pass | 83.7 | 69.0 | Y | |
| 189 | [M + 2H]2+ | 642.3 | [M + 3H]3+ | 1924.23 | 2380.23 | pass | 91.7 | 70.0 | Y | |
| 190 | [M + 2H]2+ | 631.6 | [M + 3H]3+ | 2892.28 | 2462.28 | pass | 86.1 | 67.0 | Y | |
| 191 | [M + 2H]2+ | 620.7 | [M + 3H]3+ | 1855.26 | 2315.26 | pass | 87.6 | 66.0 | Y | |
| 192 | [M + 2H]2+ | 616.3 | [M + 3H]3+ | 1846.25 | 2416.25 | pass | 82.7 | 65.0 | Y | |
| 193 | [M + 2H]2+ | — | — | 1723.13 | 2065.13 | pass | 94.0 | 66.0 | Y | |
| 194 | [M + 2H]2+ | 600.0 | [M + 3H]3+ | 1787.12 | 2253.12 | pass | 85.9 | 86.0 | Y | |
| 195 | [M + 2H]2+ | 611.4 | [M + 3H]3+ | 1831.18 | 2287.18 | pass | 87.8 | 68.0 | Y | |
| 196 | [M + 2H]2+ | 659.7 | [M + 3H]3+ | 1976.30 | 2432.30 | pass | 90.3 | 66.0 | Y | |
| 197 | [M + 2H]2+ | — | — | 2054.39 | 2312.39 | pass | 93.9 | 66.0 | Y | |
| 198 | [M + 2H]2+ | — | — | 2070.37 | 2298.37 | pass | 84.2 | 66.0 | Y | |
| 199 | [M + 2H]2+ | 627.5 | [M + 3H]3+ | 1880.08 | 2108.08 | pass | 92.0 | 66.0 | Y | |
| 200 | [M + 2H]2+ | 589.5 | [M + 3H]3+ | 1769.98 | 1933.98 | pass | 91.4 | 66.0 | Y | |
| 201 | [M + 2H]2+ | — | — | 1556.74 | 1786.74 | pass | 84.9 | 6SXI | Y | |
| 202 | [M + 2H]2+ | — | — | 1414.62 | 1528.62 | pass | 80.4 | 66.0 | Y | |
| 203 | [M + 2H]2+ | — | — | 1445.58 | 1559.58 | pass | 83.7 | 67.0 | Y | 3) |
| 204 | [M + 2H]2+ | — | — | 1515.67 | 1743.67 | pass | 90.0 | 67.0 | Y | |
| 205 | [M + 2H]2+ | 548.3 | [M + 3H]3+ | 1641.83 | 1983.83 | pass | 88.2 | 66.0 | Y | |
| 206 | [M + 2H]2+ | 558.6 | [M + 3H]3+ | 1672.92 | 2325.92 | pass | 87.6 | 66.0 | Y | |
| 207 | [M + 2H]2+ | 574.2 | [M + 3H]3+ | 1720.11 | 2176.11 | pass | 89.1 | 68.0 | Y | |
| 208 | [M + 2H]2+ | 601.7 | [M + 3H]3+ | 1802.24 | 2486.24 | pass | 82.0 | 67.0 | Y | |
| 209 | [M + 2H]2+ | 601.7 | [M + 3H]3+ | 1800.24 | 7436.24 | pass | 82.3 | 67.0 | Y | |
| 210 | [M + 2H]2+ | 610.3 | [M + 3H]3+ | 1828.21 | 2512.21 | pass | 91.3 | 66.0 | Y | |
| 211 | [M + 2H]2+ | 494.5 | [M + 3H]3+ | 1480.79 | 2050.79 | pass | 97.3 | 68.0 | Y | |

Comments:
1) estimated on ESI/MAID:
2) estimated on MALD:
3) dissolved in ACN/H2O
4) dissolved in TFA/H2O
5) dissolved in DMSO

TABLE 7
HIV-1 Consensus B Tat (NIH AIDS Reagent 5138)
DATA SHEET
HIV-1 Consensus 8 Tat (15-mer) peptides - Complete Set (Cat# 5138, Lot# 11)

| | | | | | | | Solubility Data | | | |
|---|---|---|---|---|---|---|---|---|---|---|
| | | | | Molecular | | Peptide | | | 10% | |
| | | | | Weight | Purity | Content | | | Acetic | |
| CAT # | Peptide Name | SEQUENCE | LOT # | g/mol | [%] | [%] | Water | PBS | Acid | DMSO |
| 5113 | HIV-1 Clade B consensus tat | MEPVDPRLEPWKHPG | 070034 | 1786.85 | >80% | 90% | − | − | − | + |
| 5114 | HIV-1 Clade B consensus tat | DPRLEPWKHPGSQPK | 070035 | 1770.88 | >80% | 90% | − | − | + | + |
| 5115 | HIV-1 Clade B consensus tat | EPWKHPGSQPKTACT | 070036 | 1665.77 | >80% | 90% | − | − | + | + |
| 5116 | HIV-1 Clade B consensus tat | HPGSQPKTACTNCYC | 070037 | 1608.63 | >80% | 90% | − | − | + | + |
| 5117 | HIV-1 Clade B consensus tat | QPKTACTNCYCKKCC | 070038 | 1692.67 | >80% | 80% | − | − | − | + |
| 5118 | HIV-1 Clade B consensus tat | ACINCYCKKCCFHCQ | 070039 | 1753.62 | >80% | 80% | − | + | + | + |
| 5119 | HIV-1 Clade B consensus tat | CYCKKCCFHCQVCFI | 070040 | 1826.71 | >80% | 85% | − | + | + | + |
| 5120 | HIV-1 Clade B consensus tat | KCCFHCQVCFITKGL | 070041 | 1728.78 | >80% | 90% | − | + | + | + |
| 5121 | HIV-1 Clade B consensus tat | HCQVCFITKGLGISY | 070042 | 1667.79 | >80% | 95% | − | + | + | + |
| 5122 | HIV-1 Clade B consensus tat | CFITKGLGISYGRKK | 070043 | 1669.89 | >80% | 80% | − | + | + | + |
| 5123 | HIV-1 Clade B consensus tat | KGLGISYGRKKRRQR | 070044 | 1802.04 | >80% | 70% | − | + | + | + |
| 5124 | HIV-1 Clade B consensus tat | ISYGRKKRRQRRRAP | 070045 | 1927.12 | >80% | 75% | − | − | − | + |
| 5125 | HIV-1 Clade B consensus tat | RKKRRQRRRAPQDSQ | 070046 | 1965.11 | >80% | 85% | − | + | + | + |
| 5126 | HIV-1 Clade B consensus tat | RQRRRAPQDSQTHQV | 070047 | 1861.97 | >80% | 90% | − | + | + | + |
| 5127 | HIV-1 Clade B consensus tat | RAPQDSGTHQVSLSK | 070048 | 1680.84 | >80% | 90% | − | + | + | + |
| 5128 | HIV-1 Clade B consensus tat | DSQTHQVSLSKQPAS | 070049 | 1611.77 | >80% | 85% | − | + | + | + |
| 5129 | HIV-1 Clade B consensus tat | HQVSLSKQPASGPRG | 070050 | 1618.83 | >80% | 70% | − | + | + | + |
| 5130 | HIV-1 Clade B consensus tat | LSKQPASQPRGDPTG | 070050 | 1618.83 | >80% | 70% | − | + | + | + |
| 5131 | HIV-1 Clade B consensus tat | PASQPRGDPTGPKES | 070052 | 1522.71 | >80% | 85% | − | + | + | + |
| 5132 | HIV-1 Clade B consensus tat | PRGDPTGPKESKKKV | 070053 | 1622.87 | >80% | 70% | − | − | + | + |
| 5133 | HIV-1 Clade B consensus tat | PTGPKESKKKVERET | 070054 | 1712.90 | >80% | 75% | − | − | + | + |
| 5134 | HIV-1 Clade B consensus tat | KESKKKVERETETDP | 070055 | 1802.90 | >80% | 85% | − | − | + | + |
| 5135 | HIV-1 Clade B consensus tat | KKVERETETDPVDQ | 070056 | 1544.75 | >80% | 95% | − | − | + | + |

Determination of solubility: appr. 0.25 mg of the respective peptide was incubcted with 1 ml of solvent.
Solubility was assessed by visual inspection of the resulting solution/suspension.
“+”: complete dissolution
“−”: incomplete dissolution
NOTE:
Peptides that are difficult to solubilize can almost always be dissolved in DMSO. Once a peptide is in solution, the DMSO can be slowly diluted witn aqueous medium. Care must be taken to ensure that the peptide does not begin to precipitate out of solution.

1. A method of preventing or treating a HIV infection comprising administering to a mammal in need thereof, a therapeutically effective amount of a CD8⁺ T cell vaccine composition, wherein the CD8⁺ T cell has been pre-stimulated with at least one HIV epitope, to thereby enhance a CD8⁺ T cell immune response against HIV.

2. The method of claim 1, wherein the pre-stimulation occurs ex-vivo.

3. The method of claim 1, wherein the CD8⁺ T cell has been pre-stimulated with at least two, three, four, five, six, seven, eight, nine, or ten HIV epitopes, alone or in combination, with at least one cytokine.

4. The method of claim 1, wherein the at least one HIV epitope is a subdominant, dominant epitope, or combination thereof.

5-7. (canceled)

8. The method of claim 4, wherein the at least one HIV epitope is selected from an epitope in the HIV-1 Gag, HIV-1 Nef, HIV-1 Rev, HIV-1 Tat, or HIV-1 Env, or combination thereof; or in a pool or mixture of HIV epitopes.

9-10. (canceled)

11. The method of claim 8, wherein the pool or mixture of HIV epitope is selected from the group consisting of (i) a pool or mixture of HIV-1 Gag represented by the peptide sequences set forth in Table 2, Table 4, or both; (ii) a pool or mixture of HIV-1 Nef represented by the peptide sequences set forth in Table 3; (iii) a pool or mixture of HIV-1 Rev represented by the peptide sequences set forth in Table 5; (iv) a pool or mixture of HIV-1 Tat represented by the peptide sequences set forth in Table 7; and (v) a pool or mixture of HIV-1 Env represented by the peptide sequences set forth in Table 6.

12-16. (canceled)

17. The method of claim 8, wherein the epitope in the HIV-1 Gag is selected from any one of SEQ ID NOs: 1-17, or combination thereof, and wherein said HIV-1 Gag is from proviral HIV-1 DNA in resting CD4+ T cells from mammals during the acute phase or chronic phase of infection.

18. (canceled)

19. The method of claim 1, wherein the at least one HIV epitope is synthetic, unmutated, or mutated.

20-22. (canceled)

23. The method of claim 1, wherein the CD8⁺ T cell is from CP36 or CP39.

24. The method of claim 1, wherein the CD8⁺ T cell is autologous.

25. (canceled)

26. The method of claim 3, wherein the at least one cytokine is interleukin-2 (IL-2).

27. The method of claim 1, wherein the CD8⁺ T cell to CD4⁺ T cell ratio is enhanced.

28. The method of claim 1, wherein the CD8⁺ T cell response targets latent or reactivated HIV-1 infected cells.

29. The method of claim 1, wherein the CD8⁺ T cell immune response is greater in magnitude than a CD8⁺ T cell immune response induced by administration of an unstimulated CD8⁺ T cell composition or by administration of the HIV epitope alone.

30. (canceled)

31. The method of claim 1, wherein the efficacy of the immune response against HIV results in aa reduction of the levels of HIV viral replication, wherein said reduction is a decreased in logio reductions of about 2-logs, 3-logs, 4-logs, 5-logs, 6-logs, 7-logs, 8-logs, or 9-logs; (ii) a reduction of levels of plasma HIV-1 RNA wherein said reduction of the levels of plasma HIV-1 RNA is in log₁₀ reductions of about 2-logs, 3-logs, 4-logs, 5-logs, 6-logs, 7-logs, 8-logs, or 9-logs; (iii) a reduction of levels of proviral HIV-1 DNA, wherein said reduction is a decrease of 100-, 200-, 300-, 400-, 500-, 600-, 700-, 800-, 900-, 1000-, 1500-, or 2000-fold; (iv) a reduction of the HIV-1 latent reservoir, wherein said reduction is a decrease of 100-, 200-, 300-, 400-, 500-, 600-, 700-, 800-, 900-, 1000-, 1500-, or 2000-fold when compared to the resting CD4⁺ T cell population of about 10¹² cells in any healthy or infected individual or total latently infected resting CD4⁺ T cell population of about 10⁶ to about 10⁷ cells; or (v) a delay in rebound of HIV viremia after cessation of antiretroviral therapy measured in months of about 1, 2, 3, 4, 5, 6, 7, 8, 9, 10, or 11 months, or combination thereof.

32-42. (canceled)

43. The method of claim 1, wherein the mammal is a human, and said human is afflicted with HIV-1, chronically infected with HIV-1, or acutely infected with HIV-1.

44-46. (canceled)

47. The method of claim 1, wherein the CD8⁺ T cell vaccine composition is administered to (i) a human on suppressive antiretroviral therapy; (ii) to the antiretroviral-treated human followed by antiretroviral treatment interruption; (iii) the antiretroviral-treated human in combination with latency reversing therapy.

48-49. (canceled)

50. The method of claim 1, wherein the composition is administered to the mammal more than one time over the course of treating or preventing.

51. The method of claim 1, wherein the composition is administered to the mammal in need thereof at about weeks two, three, four, five, six, seven, eight, nine, ten, eleven, twelve, thirteen, fourteen, fifteen, and sixteen post-HIV infection.

52. The method of claim 1, wherein the therapeutically effective amount is about 10², 10³, 10⁴, 10⁵, 10⁶, 10⁷, 10⁸, 10⁹, 10¹⁰, 10¹¹, or 10¹² prestimulated CD8⁺ T cells per infusion into a patient.

53. (canceled)
